(19) United States
(12) Patent Application Publication
(10) Pub. No.: US 2026/0284170 A1
(43) Pub. Date: Sep. 24, 2026

(54) Immunogenic compositions comprising conjugated capsular saccharide antigens and uses thereof

(71) Applicant: Pfizer Inc., New York, NY (US)

(72) Inventors: Kaushik DUTTA, River Edge, NJ (US); Jin-hwan KIM, Suffern, NY (US); Justin Keith MORAN, West Nyack, NY (US); Suddham SINGH, Monroe, NY (US); YuYing YANG, Stamford, CT (US)

(21) Appl. No.: 19/477,361

(22) PCT Filed: Apr. 22, 2024
(86) PCT No.: PCT/IB2024/053897

§ 371 Date: Oct. 21, 2025

Related U.S. Application Data
(60) Provisional application No. 63/497,820, filed on Apr. 24, 2023.

(51) Int. Cl. A61K 39/09; A61K 39/00; A61K 39/385; C08B 37/00

(52) U.S. Cl. CPC A61K 39/092; A61K 39/385; C08B 37/006; A61K 2039/6037

(57) ABSTRACT

The present invention relates to new immunogenic compositions comprising conjugated Streptococcus pneumoniae capsular saccharide antigens (glycoconjugates), kits comprising said immunogenic compositions and uses thereof. Immunogenic compositions of the present invention will typically comprise at least one glycoconjugate from a S. pneumoniae serotype not found in PREVNAR®, SYNFLORIX® and/or PREVNAR 13®. The invention also relates to vaccination of human subjects, in particular infants and elderly, against pneumococcal infections using said novel immunogenic compositions.

### FIELD OF THE INVENTION

The present invention relates to the field of immunogenic compositions and vaccines, their manufacture, and the use of such compositions in medicine.

More particularly, it relates to isolated Streptococcus pneumoniae serotype 22A saccharides, glycoconjugates thereof, methods for making Streptococcus pneumoniae serotype 22A glycoconjugates and immunogenic composition comprising a Streptococcus pneumoniae serotype 22A glycoconjugate.

The invention also relates to analytical methods to analyze isolated S. pneumoniae serotype 22A polysaccharide, reduced serotype 22A polysaccharide or Streptococcus pneumoniae serotype 22A glycoconjugates.

The Streptococcus pneumoniae serotype 22A saccharide and glycoconjugates of the invention can be used as a vaccine.

### BACKGROUND OF THE INVENTION

The approach to increasing immunogenicity of poorly immunogenic molecules by conjugating these molecules to “carrier” molecules has been utilized successfully for decades (see, e.g., Goebel et al. (1939) J. Exp. Med. 69:53). For example, many immunogenic compositions have been described in which purified capsular polymers have been conjugated to carrier proteins to create more effective immunogenic compositions by exploiting this “carrier effect.” Schneerson et al. (1984) Infect. Immun. 45:582-591). Conjugation has also been shown to bypass the poor antibody response usually observed in infants when immunized with a free polysaccharide (Anderson et al. (1985) J. Pediatr. 107:346; Insel et al. (1986) J. Exp. Med. 158:294).

Conjugates have been successfully generated using various cross-linking or coupling reagents, such as homobifunctional, heterobifunctional, or zero-length crosslinkers. Many methods are currently available for coupling immunogenic molecules, such as saccharides, proteins, and peptides, to peptide or protein carriers. Most methods create amine, amide, urethane, isothiourea, or disulfide bonds, or in some cases thioethers. A disadvantage to the use of cross-linking or coupling reagents which introduce reactive sites into the side chains of reactive amino acid molecules on carrier and/or immunogenic molecules is that the reactive sites, if not neutralized, are free to react with any unwanted molecule either in vitro (thus potentially adversely affecting the functionality or stability of the conjugates) or in vivo (thus posing a potential risk of adverse events in persons or animals immunized with the preparations). Such excess reactive sites can be reacted or “capped”, so as to inactivate these sites, utilizing various known chemical reactions, but these reactions may be otherwise disruptive to the functionality of the conjugates.

Thus, there remains a need for new glycoconjugates appropriately capped and methods to prepare said conjugates, such that the functionality is preserved, and the conjugate retains the ability to elicit the desired immune response.

Pneumococcal polysaccharides, in particular capsular polysaccharides, are important immunogens found on the surface of the bacteria. This has led to them being an important component in the design of pneumococcal vaccines. They have proved useful in eliciting immune responses especially when linked to carrier proteins.

Thus, there is a need for antigens which are able to generate a robust immune response to Streptococcus pneumoniae serotype 22A.

The present invention provides in particular Streptococcus pneumoniae serotype 22A glycoconjugates.

### SUMMARY OF THE INVENTION

To meet these and other needs, the present invention relates an isolated S. pneumoniae serotype 22A saccharide with the following repeating unit:

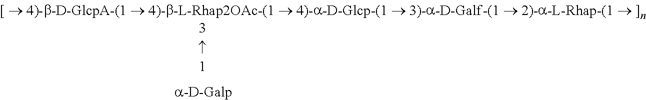

where n represents the number of repeating units.

The invention further relates to an isolated S. pneumoniae serotype 22A saccharide with the following repeating unit:

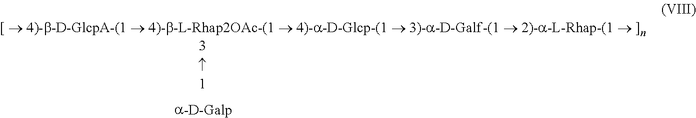

where n represents the number of repeating units and wherein the O-acetyl group at position 2 of β-D-Rhap is present in about 50% to about 100% of the repeating units.

The invention further pertains to an isolated S. pneumoniae serotype 22A saccharide with the following repeating unit:

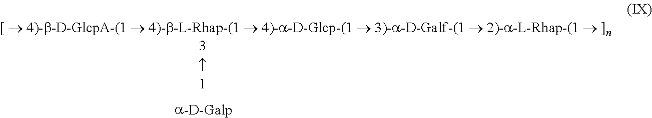

where n represents the number of repeating units.

In an aspect, the invention pertains to a glycoconjugate comprising an isolated S. pneumoniae serotype 22A saccharide as specified above conjugated to a carrier protein.

In an aspect, the invention pertains to a serotype 22A glycoconjugate comprising a serotype 22A capsular saccharide comprising between about 1 to about 70 α-L-Arabinofuranose (α-L-Araf) residues in every 100 saccharide repeat units of the saccharide.

The invention further pertains to a serotype 22A glycoconjugate comprising a serotype 22A capsular saccharide with the following repeating unit:

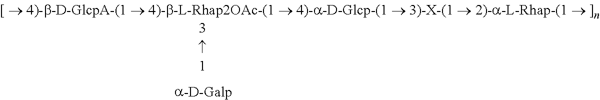

where n represents the number of repeating units, wherein X represents either a α-D-galactofuranose (α-D-Galf) or a α-L-Arabinofuranose (α-L-Araf) residue.

The invention also pertains to an immunogenic composition comprising said S. pneumoniae serotype 22A saccharide and/or said S. pneumoniae serotype 22A saccharide glycoconjugate.

The invention also pertains to said immunogenic composition for use as a vaccine.

The invention further pertains to analytical methods such as a method of detecting the presence of O-acetyl groups in an isolated S. pneumoniae serotype 22A polysaccharide, a method of detecting the presence of O-acetyl groups in a reduced serotype 22A polysaccharide, a method of detecting the presence of O-acetyl groups in S. pneumoniae serotype 22A glycoconjugate.

The invention also pertains to a method of measuring the degree of O-acetylation of an isolated S. pneumoniae serotype 22A polysaccharide, a method of measuring the degree of O-acetylation in S. pneumoniae serotype 22A glycoconjugate, said method comprising the step of: a) preparing a S. pneumoniae serotype 22A glycoconjugate and b) measuring the amount of O-acetyl groups in said glycoconjugate.

The invention further pertains to a method of detecting the presence of L-Arabinofuranose-5-Aldehyde residues in an oxidized serotype 22A polysaccharide, said method comprising the step of: a) reacting an isolated S. pneumoniae serotype 22A polysaccharide with an oxidized agent and b) detecting the presence of L-Arabinofuranose-5-Aldehyde residues in said oxidized polysaccharide.

The invention also pertains to a method of detecting the presence of α-L-Arabinofuranose (α-L-Araf) residues in a reduced serotype 22A polysaccharide, said method comprising the step of: a) reacting an isolated S. pneumoniae serotype 22A polysaccharide with a reducing agent and b) detecting the presence of α-L-Arabinofuranose (α-L-Araf) residues in said reduced polysaccharide.

In an aspect, the invention pertains to a method of detecting the presence of α-L-Arabinofuranose (α-L-Araf) residues in S. pneumoniae serotype 22A glycoconjugate, said method comprising the step of: a) preparing a S. pneumoniae serotype 22A glycoconjugate and b) detecting the presence of α-L-Arabinofuranose (α-L-Araf) residues in said glycoconjugate.

### DETAILED DESCRIPTION OF THE DRAWINGS

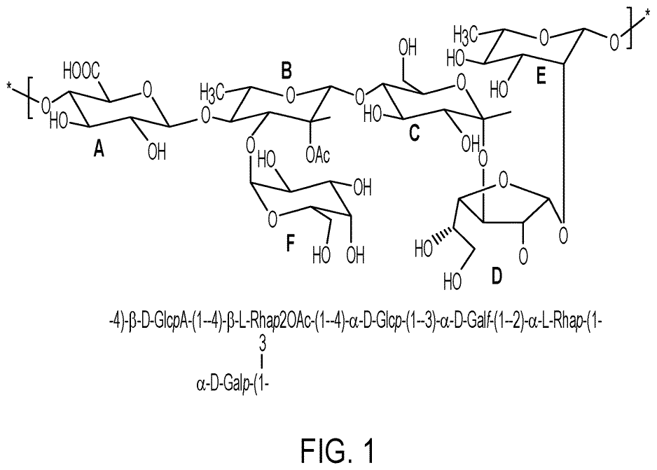

FIG. 1. Schematic of pneumococcal polysaccharide serotype 22A repeat unit organization.

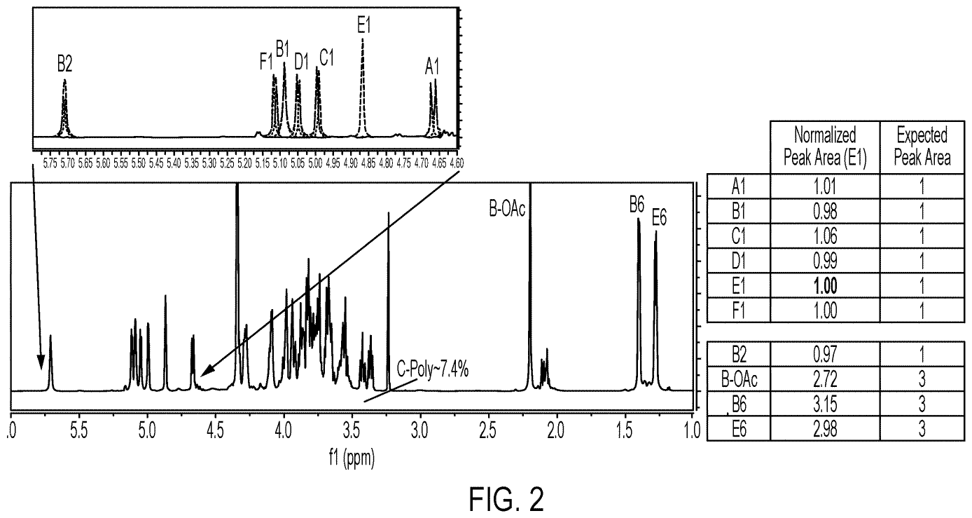

FIG. 2. 1D ¹H spectrum of serotype 22A polysaccharide (insert spectrum shows the expanded anomeric region). Inset table shows the normalized and expected peak area for the anomeric and methyl protons. The anomeric and the methyl signals are annotated.

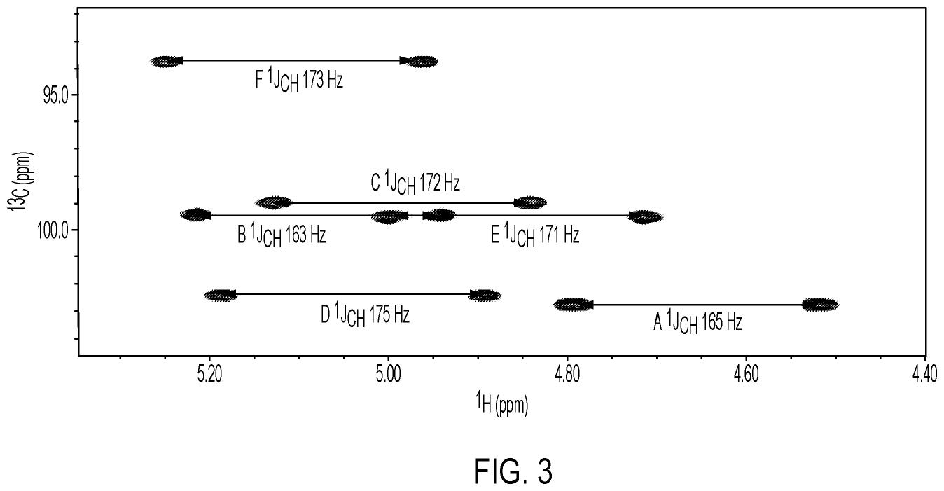

FIG. 3. The expanded anomeric region of proton decoupled ¹H-¹³C HSQC spectrum of serotype 22A polysaccharide. The coupling constant ¹J_{CH} for each of the sugars is annotated. Table 1 below lists the ¹J_{CH} and ³J_{HH} coupling constants for each sugar in serotype 22A.

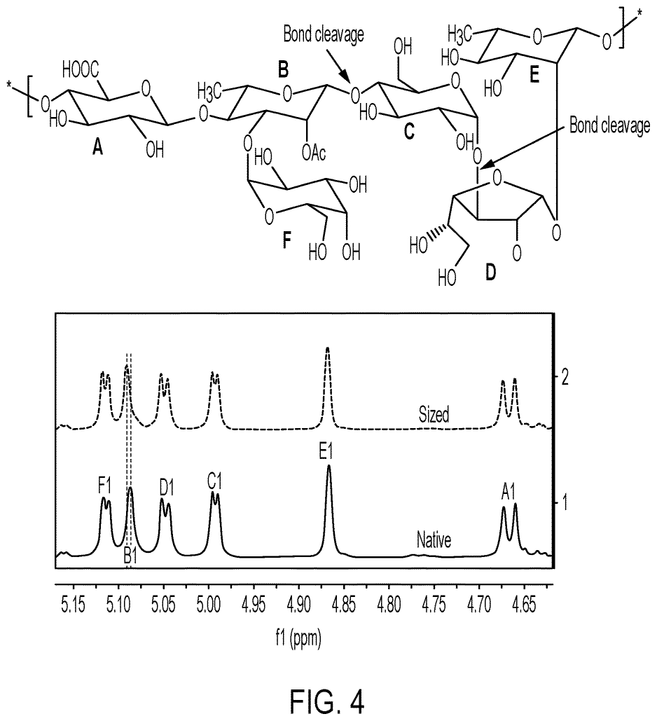

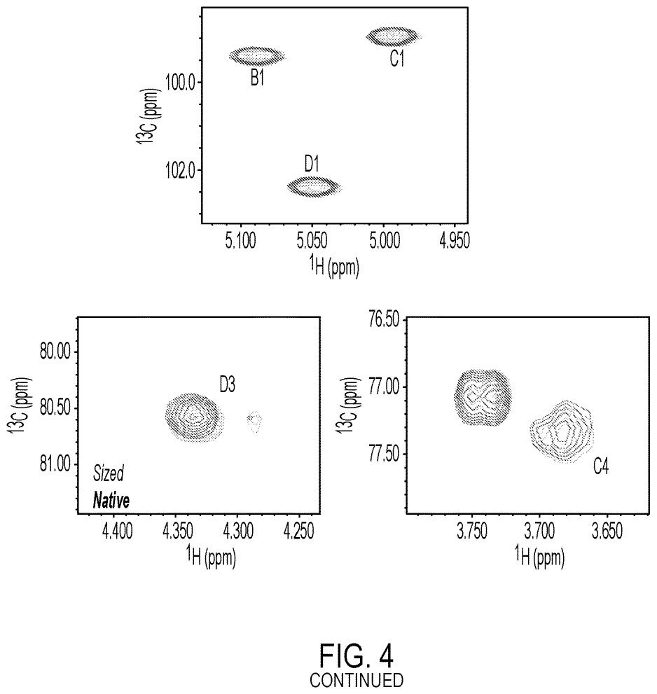

FIG. 4. 1D and 2D spectra of native (lower spectra) and sized (upper spectra) serotype 22A polysaccharide. Linea in the 1D spectra panel shows the slight shift in the B1 resonances. 2D HSQC spectra show the slight shift in the D3 and C4 resonances.

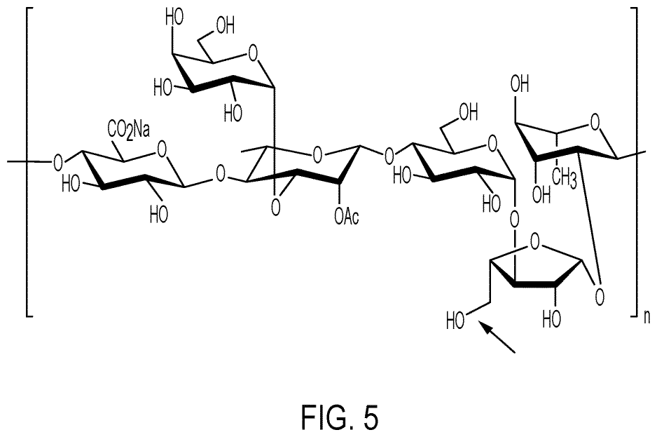

FIG. 5. Schematic of a pneumococcal polysaccharide serotype 22A repeat unit following treatment with an oxidizing agent such as periodate, followed with treatment by a reducing agent such as NaBH₄. The reducing agent reduces the oxidized α-D-Galf residue from a ketone/hydrate to an alcohol and transform the oxidized α-D-Galf to α-L-Arabinofuranose (α-L-Araf) residue.

### DETAILED DESCRIPTION OF THE INVENTION

The present invention is based, in part, on the identification of novel pneumococcal polysaccharide structure(s) by using NMR spectroscopy. It is believed that the structure provided herein is the first identification or the first correct identification of S. pneumoniae serotype 22A. The produced (and purified) polysaccharide was used to generate polysaccharide-protein conjugate (glycoconjugates). S. pneumoniae serotype 22A has a unique polysaccharide structure, which results in unique consideration when designing conjugate production process. The S. pneumoniae serotype 22A glycoconjugates of the invention also have a unique structure and design.

1. Isolated Streptococcus pneumoniae Serotype 22A Saccharide of the Invention

As used herein, the term “isolated” in connection with a saccharide refers to isolation of S. pneumoniae serotype specific capsular polysaccharide from purified polysaccharide using purification techniques known in the art, including the use of centrifugation, depth filtration, precipitation, ultrafiltration, treatment with activate carbon, diafiltration and/or column chromatography. Generally, an isolated polysaccharide refers to partial removal of proteins, nucleic acids and non-specific endogenous polysaccharide (C-polysaccharide). The isolated polysaccharide contains less than 10%, 8%, 6%, 4%, or 2% protein impurities and/or nucleic acids. The isolated polysaccharide contains less than 20% of C-polysaccharide with respect to type specific polysaccharides.

The term “saccharide” throughout this specification may indicate polysaccharide or oligosaccharide and includes both. In preferred embodiments, the saccharide is a polysaccharide, in particular a S. pneumoniae serotype 22A capsular polysaccharide.

The term “serotype 22A saccharide”, “serotype 22A capsular saccharide”, “S. pneumoniae serotype 22A saccharide” throughout this specification refers to S. pneumoniae serotype 22A capsular saccharide and may be used interchangeably herein.

The structure of S. pneumoniae serotype 22A capsular polysaccharide is disclosed for the first time and is shown in FIG. 1. The inventors found that the serotype 22A polysaccharide is a hexa-saccharide: β-D-glucuronic acid (A), O-acetylated β-L-rhamnose (B), α-D-Glucose (C), α-D-galactofuranose (D), α-D-galactopyranose (F) and α-L-rhamnose (E). The main chain consists of five sugars and one branched sugar linked to 3 position of O-acetylated β-L-rhamnose (B).

Accordingly, in one embodiment, the present invention provides an isolated S. pneumoniae serotype 22A saccharide with the following repeating unit:

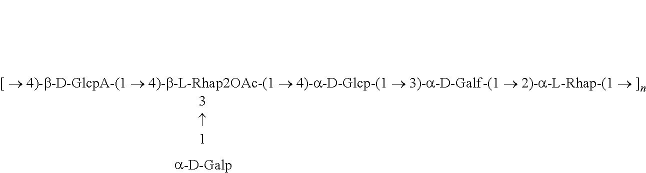

where n represents the number of repeating units.

As shown at FIG. 1, β-L-rhamnose (residue B) of serotype 22A polysaccharide is O-acetylated at carbon 2 position. In an embodiment, all the repeating unit of serotype 22A polysaccharide is O-acetylated at carbon 2 position of the β-L-rhamnose residue.

Therefore, in one embodiment, the present invention provides an isolated S. pneumoniae serotype 22A saccharide with the following repeating unit:

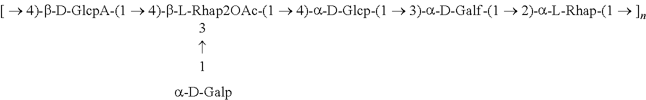

where n represents the number of repeating units and wherein the O-acetyl group at position 2 of β-D-Rhap is present in about 100% of the repeating units.

O-acetylation level may however vary from one strain to another. Therefore, in one embodiment, the present invention provides an isolated S. pneumoniae serotype 22A saccharide with the following repeating unit:

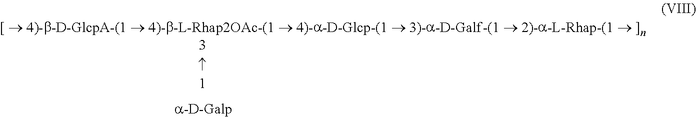

where n represents the number of repeating units and wherein the O-acetyl group at position 2 of β-D-Rhap is present in about 50% to about 100% of the repeating units.

In one embodiment, the present invention provides an isolated S. pneumoniae serotype 22A saccharide with the repeating unit of formula (VIII), where n represents the number of repeating units and wherein the O-acetyl group at position 2 of β-D-Rhap is present in about 60% to about 95% of the repeating units.

In one embodiment, the present invention provides an isolated S. pneumoniae serotype 22A saccharide with the repeating unit of formula (VIII), where n represents the number of repeating units and wherein the O-acetyl group at position 2 of β-D-Rhap is present in about 80% to about 99% of the repeating units.

Furthermore, native S. pneumoniae serotype 22A saccharide can be deacetylated, for example by treatment with a base (alkaline pH).

Therefore, in one embodiment, the present invention provides an isolated S. pneumoniae serotype 22A saccharide with the following repeating unit:

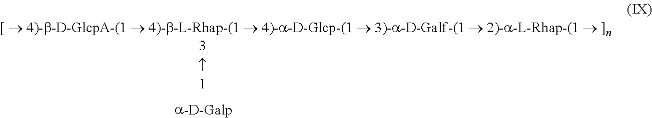

where n represents the number of repeating units.

The native S. pneumoniae serotype 22A saccharide may be partially deacetylated, for example by gentle treatment with a base (alkaline pH).

Accordingly, in one embodiment, the present invention provides an isolated S. pneumoniae serotype 22A saccharide with the repeating unit of formula (VIII), where n represents the number of repeating units, and wherein the O-acetyl group at position 2 of β-D-Rhap is present in about 0% to about 95% of the repeating units.

In one embodiment, the present invention provides an isolated S. pneumoniae serotype 22A saccharide with the repeating unit of formula (VIII), where n represents the number of repeating units, and wherein the O-acetyl group at position 2 of β-D-Rhap is present in about 10% to about 90% of the repeating units.

In one embodiment, the present invention provides an isolated S. pneumoniae serotype 22A saccharide with the repeating unit of formula (VIII), where n represents the number of repeating units, and wherein the O-acetyl group at position 2 of β-D-Rhap is present in about 50% to about 80% of the repeating units.

In certain embodiments, the isolated S. pneumoniae serotype 22A saccharide of the invention has between 10 and 5,000 repeating units. In certain aspects, the isolated saccharide has between 50 and 4,500 repeating units. In certain aspects, the isolated saccharide has between 100 and 4,500 repeating units. In certain aspects, the isolated saccharide has between 150 and 2,000 repeating units.

Isolated capsular saccharides from S. pneumoniae serotype 22A can be prepared by standard techniques known to those of ordinary skill in the art. Typically capsular polysaccharides are produced by growing a S. pneumoniae serotype 22A strain in a medium (e.g., in a soy-based medium), the polysaccharides are then prepared from the bacteria culture. Serotype 22A Streptococcus pneumoniae strains may be obtained from established culture collections (such as for example the Streptococcal Reference Laboratory (Centers for Disease Control and Prevention, Atlanta, GA)) or clinical specimens.

The population of the organism (S. pneumoniae serotype 22A) is often scaled up from a seed vial to seed bottles and passaged through one or more seed fermentors of increasing volume until production scale fermentation volumes are reached. At the end of the growth cycle the cells are lysed and the lysate broth is then harvested for downstream (purification) processing (see for example WO 2006/110381 and WO 2008/118752, U.S. Patent App. Pub. Nos. 2006/0228380, 2006/0228381, 2008/0102498 and US2008/0286838). The polysaccharides are typically purified through centrifugation, precipitation, ultra-filtration, and/or column chromatography (see for example WO 2006/110352, WO 2008/118752 and WO2020/170190).

The isolated polysaccharide can be characterized by different parameters including, for example the weight average molecular weight (Mw). The molecular weight of the polysaccharide can be measured by Size Exclusion Chromatography (SEC) combined with Multiangle Laser Light Scattering detector (MALLS).

In an embodiment, the isolated S. pneumoniae serotype 22A saccharide of the invention has a weight average molecular weight between 5 kDa and 5000 kDa. In an embodiment, the isolated S. pneumoniae serotype 22A saccharide has a weight average molecular weight between 5 kDa and 2000 kDa.

In an embodiment, the isolated S. pneumoniae serotype 22A polysaccharide has a weight average molecular weight between 50 kDa and 5000 kDa. In an embodiment, the isolated S. pneumoniae serotype 22A polysaccharide has a weight average molecular weight between 50 kDa and 2000 kDa. In an embodiment, the isolated S. pneumoniae serotype 22A polysaccharide has a weight average molecular weight between 50 kDa and 1000 kDa.

In an embodiment, the isolated S. pneumoniae serotype 22A polysaccharide has a weight average molecular weight between 100 kDa and 5000 kDa. In an embodiment, the isolated S. pneumoniae serotype 22A polysaccharide has a weight average molecular weight between 100 kDa and 2000 kDa. In an embodiment, the isolated S. pneumoniae serotype 22A polysaccharide has a weight average molecular weight between 100 kDa and 1000 kDa. In an embodiment, the isolated S. pneumoniae serotype 22A polysaccharide has a weight average molecular weight between 100 kDa and 500 kDa.

In an embodiment, the isolated S. pneumoniae serotype 22A polysaccharide has a weight average molecular weight between 300 kDa and 5000 kDa. In an embodiment, the isolated S. pneumoniae serotype 22A polysaccharide has a weight average molecular weight between 300 kDa and 2000 kDa. In an embodiment, the isolated S. pneumoniae serotype 22A polysaccharide has a weight average molecular weight between 300 kDa and 1000 kDa.

In an embodiment, the isolated S. pneumoniae serotype 22A polysaccharide has a weight average molecular weight between 500 kDa and 3000 kDa. In an embodiment, the isolated polysaccharide has a weight average molecular weight between 500 kDa and 2000 kDa. In an embodiment, the isolated polysaccharide has a weight average molecular weight between 500 kDa and 1000 kDa.

Preferably, in order to generate glycoconjugates with advantageous filterability characteristics, immunogenicity and/or yields, sizing of the saccharide to a target molecular weight range is performed prior to the conjugation to a carrier protein.

Advantageously, the size of the purified capsular S. pneumoniae serotype 22A saccharide is reduced while preserving critical features of the structure of the polysaccharide. Mechanical or chemical sizing maybe employed.

In an embodiment, the size of the purified S. pneumoniae serotype 22A capsular saccharide is reduced by chemical hydrolysis. Chemical hydrolysis maybe conducted using a mild acid (e.g., acetic acid, formic acid, propanoic acid). In an embodiment, chemical hydrolysis is conducted using formic acid. In an embodiment, chemical hydrolysis is conducted using propanoic acid. In a preferred embodiment, chemical hydrolysis is conducted using acetic acid. Chemical hydrolysis may also be conducted using a diluted strong acid (such as diluted hydrochloric acid, diluted sulfuric acid, diluted phosphoric acid, diluted nitric acid or diluted perchloric acid). In an embodiment, chemical hydrolysis is conducted using diluted hydrochloric acid. In an embodiment, chemical hydrolysis is conducted using diluted sulfuric acid. In an embodiment, chemical hydrolysis is conducted using diluted phosphoric acid. In an embodiment, chemical hydrolysis is conducted using diluted nitric acid. In an embodiment, chemical hydrolysis is conducted using diluted perchloric acid.

The size of the purified S. pneumoniae serotype 22A capsular saccharide can also be reduced by mechanical homogenization. In an embodiment, the size of the purified capsular saccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

The high-pressure homogenization process can be appropriate for reducing the size of the purified capsular saccharide while preserving the structural features of the saccharide.

In a preferred embodiment, the isolated S. pneumoniae serotype 22A capsular saccharide is sized to a weight average molecular weight between 10 kDa and 1000 kDa. In an embodiment, the isolated S. pneumoniae serotype 22A capsular saccharide is sized to a weight average molecular weight between 50 kDa and 500 kDa. In an embodiment, the isolated S. pneumoniae serotype 22A capsular saccharide is sized to a weight average molecular weight between 50 kDa and 400 kDa. In an embodiment, the isolated S. pneumoniae serotype 22A capsular saccharide is sized to a weight average molecular weight between 50 kDa and 250 kDa.

In a preferred embodiment, the isolated S. pneumoniae serotype 22A capsular saccharide is sized to a weight average molecular weight between 100 kDa and 1000 kDa. In an embodiment, the isolated S. pneumoniae serotype 22A capsular saccharide is sized to a weight average molecular weight between 100 kDa and 500 kDa. In an embodiment, the isolated S. pneumoniae serotype 22A capsular saccharide is sized to a weight average molecular weight between 100 kDa and 400 kDa. In an embodiment, the isolated S. pneumoniae serotype 22A capsular saccharide is sized to a weight average molecular weight between 100 kDa and 250 kDa.

In an embodiment, the isolated S. pneumoniae serotype 22A capsular saccharide is sized to a weight average molecular weight between 250 kDa and 1000 kDa. In an embodiment, the isolated S. pneumoniae serotype 22A capsular saccharide is sized to a weight average molecular weight between 250 kDa and 500 kDa. In an embodiment, the isolated S. pneumoniae serotype 22A capsular saccharide is sized to a weight average molecular weight between 250 kDa and 400 kDa. In a preferred embodiment, the isolated S. pneumoniae serotype 22A capsular saccharide is sized to a weight average molecular weight between 200 kDa and 800 kDa.

In an embodiment, the isolated S. pneumoniae serotype 22A capsular saccharide capsular saccharide is sized to a weight average molecular weight of about 250 kDa. In an embodiment, the isolated S. pneumoniae serotype 22A capsular saccharide is sized to a weight average molecular weight of about 300 kDa. In an embodiment, the isolated S. pneumoniae serotype 22A capsular saccharide is sized to a weight average molecular weight of about 350 kDa. In an embodiment, the isolated S. pneumoniae serotype 22A capsular saccharide is sized to a weight average molecular weight of about 400 kDa. In an embodiment, the isolated S. pneumoniae serotype 22A capsular saccharide is sized to a weight average molecular weight of about 450 kDa. In an embodiment, the isolated S. pneumoniae serotype 22A capsular saccharide is sized to a weight average molecular weight of about 500 kDa. In an embodiment, the isolated S. pneumoniae serotype 22A capsular saccharide is sized to a weight average molecular weight of about 550 kDa. In an embodiment, the isolated S. pneumoniae serotype 22A capsular saccharide is sized to a weight average molecular weight of about 600 kDa. In an embodiment, the isolated S. pneumoniae serotype 22A capsular saccharide is sized to a weight average molecular weight of about 700 kDa. In an embodiment, the isolated S. pneumoniae serotype 22A capsular saccharide is sized to a weight average molecular weight of about 800 kDa. In an embodiment, the isolated S. pneumoniae serotype 22A capsular saccharide is sized to a weight average molecular weight of about 900 kDa. In an embodiment, the isolated S. pneumoniae serotype 22A capsular saccharide is sized to a weight average molecular weight of about 1000 kDa.

In an embodiment, the isolated capsular saccharide is not sized.

The isolated capsular saccharide described above may be activated (e.g., chemically activated) to make them capable of reacting and then incorporated into glycoconjugates, as further described herein.

2. Streptococcus pneumoniae Serotype 22A Glycoconjugates of the Invention

For the purpose of the invention the term ‘glycoconjugate’ indicates a capsular saccharide conjugated to a carrier protein via covalent or non-covalent bonds. In an embodiment, the capsular saccharide is conjugated to a carrier protein via non-covalent bonds (such as the rhizavidin/biotin system, see e.g. WO2012155007, WO2020056202). Preferably, the capsular saccharide is conjugated via covalent bonds. In one embodiment the capsular saccharide is conjugated directly to a carrier protein. In a second embodiment the capsular saccharide is conjugated to a carrier protein through a spacer/linker.

The present invention provides glycoconjugates in which saccharides as provided for above are conjugated to a carrier protein. Therefore, in an embodiment, the invention provides a glycoconjugate comprising a saccharide having the above disclosed repeating unit conjugated to a carrier protein.

In an embodiment, the invention provides a glycoconjugate consisting of a saccharide having the above disclosed repeating unit conjugated to a carrier protein.

2.1 Attributes of the Streptococcus pneumoniae Serotype 22A Glycoconjugates of the Invention

The isolated polysaccharide described above may be activated (e.g., chemically activated) to make them capable of reacting (e.g. with a linker or directly with the carrier protein) and then incorporated into glycoconjugates, as further described herein.

Before activation, the size of the isolated polysaccharide can be reduced while preserving critical features of the structure of the polysaccharide. Mechanical or chemical sizing maybe employed. In an embodiment, the size of the isolated polysaccharide is reduced by chemical hydrolysis. The size of the isolated polysaccharide can also be reduced by mechanical homogenization. In an embodiment, the size of the isolated polysaccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

The weight average molecular weight (Mw) of the saccharide before conjugation refers to the Mw before the activation of the saccharide (i.e. after an eventual sizing step but before reacting the saccharide with an activating agent). In the context of the present invention the Mw of the saccharide is not substantially modified by the activation step and the Mw of the saccharide incorporated in the conjugate is similar to the Mw of the saccharide as measured before activation.

In an embodiment, the serotype 22A glycoconjugate of the present invention comprises a serotype 22A polysaccharide wherein the weight average molecular weight (Mw) of said polysaccharide before conjugation is between 50 kDa and 1,000 kDa.

In an embodiment, the serotype 22A glycoconjugate of the present invention comprises a serotype 22A polysaccharide wherein the weight average molecular weight (Mw) of said polysaccharide before conjugation is between 100 kDa and 600 kDa.

In an embodiment, the serotype 22A glycoconjugate of the present invention comprises a serotype 22A polysaccharide wherein the weight average molecular weight (Mw) of said polysaccharide before conjugation is between 100 kDa and 400 kDa.

In an embodiment, the serotype 22A glycoconjugate of the present invention comprises a serotype 22A polysaccharide wherein the weight average molecular weight (Mw) of said polysaccharide before conjugation is between 150 kDa and 300 kDa.

In some embodiments, the serotype 22A glycoconjugate of the invention has a weight average molecular weight (Mw) of between 250 kDa and 20,000 kDa.

In other embodiments, the serotype 22A glycoconjugate has a weight average molecular weight (Mw) of between 500 kDa and 15,000 kDa.

In other embodiments, the serotype 22A glycoconjugate has a weight average molecular weight (Mw) of between 500 kDa and 10,000 kDa.

In still other embodiments, the serotype 22A glycoconjugate has a weight average molecular weight (Mw) of between 750 kDa and 7,500 kDa.

In preferred embodiments, the serotype 22A glycoconjugate has a weight average molecular weight (Mw) of between 1,000 kDa and 5,000 kDa.

Native serotype 22A polysaccharide is O-acetylated and the total amount of O-acetylation is approximately one O-acetyl group per polysaccharide repeating unit. The degree of O-acetylation of the polysaccharide can be determined by any method known in the art, for example, by proton NMR or by ion-HPLC analysis (see for example Lemercinier et al. (1996) Carbohydrate Research 296:83-96; Jones et al. (2002) J. Pharmaceutical and Biomedical Analysis 30:1233-1247; Hestrin, S. (1949) J. Biol. Chem. 180:249-261).

In an embodiment, the serotype 22A glycoconjugate of the present invention comprises a serotype 22A saccharide comprising on average at least 0.5 O-acetyl group per saccharide repeating unit. In a preferred embodiment, the serotype 22A glycoconjugate of the present invention comprises a serotype 22A saccharide comprising on average at least 0.8 O-acetyl group per saccharide repeating unit.

In a preferred embodiment, the serotype 22A glycoconjugate of the present invention comprises a serotype 22A saccharide comprising on average between about 0.8 and about 1 O-acetyl group per saccharide repeating unit.

In an embodiment, the ratio of O-acetyl group per saccharide repeating unit in the glycoconjugate to O-acetyl group per saccharide repeating unit in the isolated polysaccharide is at least 0.6. In a preferred embodiment, the ratio of O-acetyl group per saccharide repeating unit in the glycoconjugate to O-acetyl group per saccharide repeating unit in the isolated polysaccharide is at least 0.9.

Another way to characterize the serotype 22A glycoconjugates of the invention is by the number of lysine residues in the carrier protein (e.g., CRM₁₉₇, SCP, DT or TT) that become conjugated to the saccharide which can be characterized as a range of conjugated lysines (degree of conjugation). The evidence for lysine modification of the carrier protein, due to covalent linkages to the polysaccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the serotype 22A glycoconjugate of the invention is between 2 and 15. In an embodiment, the degree of conjugation of the serotype 22A glycoconjugate of the invention is between 2 and 10. In an embodiment, the degree of conjugation of the serotype 22A glycoconjugate of the invention is between 3 and 5. In an embodiment, the degree of conjugation of the serotype 22A glycoconjugate of the invention is between 2 and 6. In an embodiment, the degree of conjugation of the serotype 22A glycoconjugate of the invention is between 4 and 10.

The serotype 22A glycoconjugates of the invention may also be characterized by the ratio (weight/weight) of saccharide to carrier protein. In some embodiments, the ratio of serotype 22A saccharide to carrier protein in the glycoconjugate (w/w) is between 0.5 and 3.0. In other embodiments, the saccharide to carrier protein ratio (w/w) is between 0.5 and 2.0. In other embodiments, the saccharide to carrier protein ratio (w/w) is between 0.5 and 1.5. In other embodiments, the saccharide to carrier protein ratio (w/w) is between 0.8 and 1.2. In other embodiments, the saccharide to carrier protein ratio (w/w) is between 0.5 and 1.0. In other embodiments, the saccharide to carrier protein ratio (w/w) is between 1.0 and 1.5. In other embodiments, the saccharide to carrier protein ratio (w/w) is between 1.0 and 2.0. In further embodiments, the saccharide to carrier protein ratio (w/w) is between 0.8 and 1.2. In a preferred embodiment, the ratio of serotype 22A saccharide to carrier protein in the conjugate is between 0.7 and 1.1. In some such embodiments, the carrier protein is CRM₁₉₇.

The serotype 22A glycoconjugates and immunogenic compositions of the invention may contain free saccharide that is not conjugated to the carrier protein but is nevertheless present in the glycoconjugate composition. The free saccharide may be noncovalently associated with (i.e., noncovalently bound to, adsorbed to, or entrapped in or with) the glycoconjugate. In a preferred embodiment, the serotype 22A glycoconjugate comprises less than about 50% of free serotype 22A saccharide compared to the total amount of serotype 22A saccharide. In a preferred embodiment, the serotype 22A glycoconjugate comprises less than about 25% of free serotype 22A saccharide compared to the total amount of serotype 22A saccharide. In an even preferred embodiment, the serotype 22A glycoconjugate comprises less than about 20% of free serotype 22A saccharide compared to the total amount of serotype 22A saccharide. In a yet preferred embodiment, the serotype 22A glycoconjugate comprises less than about 15% of free serotype 22A saccharide compared to the total amount of serotype 22A saccharide.

The serotype 22A glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to K_{d} by the expression, K_{d}=(Vₑ−V₀)/(Vᵢ−V₀).

In a preferred embodiment, at least 30% of the serotype 22A glycoconjugate has a K_{d} below or equal to 0.3 in a CL-4B column. In a preferred embodiment, at least 40% of the glycoconjugate has a K_{d} below or equal to 0.3 in a CL-4B column. In a preferred embodiment, at least 60% of the serotype 22A glycoconjugate has a K_{d} below or equal to 0.3 in a CL-4B column. In a preferred embodiment, between 50% and 80% of the serotype 22A glycoconjugate has a K_{d} below or equal to 0.3 in a CL-4B column. In a preferred embodiment, between 65% and 80% of the serotype 22A glycoconjugate has a K_{d} below or equal to 0.3 in a CL-4B column.

2.2 Streptococcus pneumoniae Serotype 22A Glycoconjugates of the Invention Bearing α-L-Arabinofuranose Residues

The process to prepare the serotype 22A glycoconjugate of the invention may comprise an oxidation step of the polysaccharide by an oxidizing agent which oxidizes terminal hydroxyl group to an aldehyde. This is the case when direct reductive amination is used. The oxidizing agent may be for example periodate. When a polysaccharide reacts with periodate, periodate oxidises vicinal hydroxyl groups to form carbonyl or aldehyde groups and causes cleavage of a C—C bond. The activated 22A polysaccharide is then compounded with a carrier protein and reacted with a reducing agent to form a glycoconjugate. In a preferred embodiment, the reducing agent is sodium cyanoborohydride. At the end of the reduction reaction, the unreacted aldehyde groups remaining in the conjugates, are usually capped using a suitable capping agent. In one embodiment this capping agent is sodium borohydride (NaBH₄).

The primary site for oxidation with periodate is the α-D-Galactofuranose (α-D-Galf) residue (residue D at FIG. 1). The activated α-D-Galf residues that don't react with the carrier protein are sensitive to reduction using NaBH₄. Treatment of activated serotype 22A polysaccharide with a reducing agent such as NaBH₄ will reduces the oxidized α-D-Galf residue from a ketone/hydrate to an alcohol and transform the oxidized α-D-Galf to α-L-Arabinofuranose (α-L-Araf) residues as illustrated in FIG. 5.

Therefore, in an embodiment, the serotype 22A glycoconjugate of the present invention comprises a serotype 22A capsular saccharide comprising between about 1 to about 70 α-L-Arabinofuranose (α-L-Araf) residues in every 100 saccharide repeat units of the saccharide. In an embodiment, the serotype 22A glycoconjugate of the present invention comprises a serotype 22A capsular saccharide comprising between about 1 to about 60 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 1 to about 50 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 1 to about 40 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 1 to about 30 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 1 to about 20 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 1 to about 10 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 1 to about 5 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 5 to about 70 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 5 to about 60 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 5 to about 50 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 5 to about 40 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 5 to about 30 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 5 to about 20 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 5 to about 10 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 10 to about 70 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 10 to about 60-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 10 to about 50 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 10 to about 40 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 10 to about 30 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising between about 10 to about 20 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising about 1 α-L-Arabinofuranose (α-L-Araf) residue in every 100 saccharide repeat units of the saccharide.

In an embodiment, the serotype 22A glycoconjugate of the present invention comprises a serotype 22A capsular saccharide comprising about 2 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising about 3 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising about 4 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising about 5 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising about 10 α-L-Arabinofuranose (α-L-Araf) residues in every 100 saccharide repeat units of the saccharide.

In an embodiment, the serotype 22A glycoconjugate of the present invention comprises a serotype 22A capsular saccharide comprising about 20 α-L-Arabinofuranose (α-L-Araf) residues in every 100 saccharide repeat units of the saccharide.

In an embodiment, the serotype 22A glycoconjugate of the present invention comprises a serotype 22A capsular saccharide with the following repeating unit:

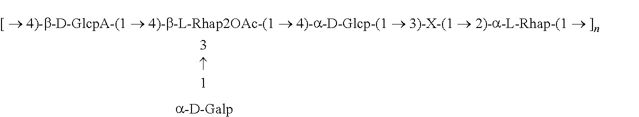

where n represents the number of repeating units, wherein X represents either a α-D-galactofuranose (α-D-Galf) or a α-L-Arabinofuranose (α-L-Araf) residue. In an embodiment, said serotype 22A capsular saccharide comprises between about 70 to about 99.5 α-D-galactofuranose (α-D-Galf) residues and between about 0.5 to about 30 α-L-Arabinofuranose (α-L-Araf) residues in every 100 saccharide repeat units of the saccharide.

In an embodiment, said serotype 22A capsular saccharide comprises between about 80 to about 99.5 α-D-galactofuranose (α-D-Galf) residues and between about 0.5 to about 20 α-L-Arabinofuranose (α-L-Araf) residues in every 100 saccharide repeat units of the saccharide.

In an embodiment, said serotype 22A capsular saccharide comprises between about 90 to about 99.5 α-D-galactofuranose (α-D-Galf) residues and between about 0.5 to about 10 α-L-Arabinofuranose (α-L-Araf) residues in every 100 saccharide repeat units of the saccharide.

In an embodiment, said serotype 22A capsular saccharide comprises between about 95 to about 99.5 α-D-galactofuranose (α-D-Galf) residues and between about 0.5 to about 5 α-L-Arabinofuranose (α-L-Araf) residues in every 100 saccharide repeat units of the saccharide.

In an embodiment, said serotype 22A capsular saccharide comprises about 90 α-D-galactofuranose (α-D-Galf) residues and about 10 α-L-Arabinofuranose (α-L-Araf) residues in every 100 saccharide repeat units of the saccharide.

In an embodiment, said serotype 22A capsular saccharide comprises about 95 α-D-galactofuranose (α-D-Galf) residues and about 5 α-L-Arabinofuranose (α-L-Araf) residues in every 100 saccharide repeat units of the saccharide.

In an embodiment, said serotype 22A capsular saccharide comprises about 96 α-D-galactofuranose (α-D-Galf) residues and about 4 α-L-Arabinofuranose (α-L-Araf) residues in every 100 saccharide repeat units of the saccharide.

In an embodiment, said serotype 22A capsular saccharide comprises about 97 α-D-galactofuranose (α-D-Galf) residues and about 3 α-L-Arabinofuranose (α-L-Araf) residues in every 100 saccharide repeat units of the saccharide.

In an embodiment, said serotype 22A capsular saccharide comprises about 98 α-D-galactofuranose (α-D-Galf) residues and about 2 α-L-Arabinofuranose (α-L-Araf) residues in every 100 saccharide repeat units of the saccharide.

In an embodiment, said serotype 22A capsular saccharide comprises about 99 α-D-galactofuranose (α-D-Galf) residues and about 1 α-L-Arabinofuranose (α-L-Araf) residues in every 100 saccharide repeat units of the saccharide.

In an embodiment, said serotype 22A capsular saccharide comprises about 99.5 α-D-galactofuranose (α-D-Galf) residues and about 0.5 α-L-Arabinofuranose (α-L-Araf) residues in every 100 saccharide repeat units of the saccharide.

In an embodiment, the serotype 22A glycoconjugate of the present invention comprises a serotype 22A capsular saccharide with the following repeating unit:

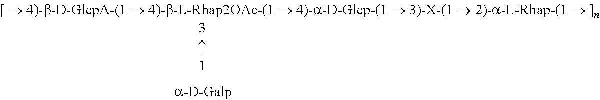

where n represents the number of repeating units, wherein X represents either a α-D-galactofuranose (α-D-Galf) or a α-L-Arabinofuranose (α-L-Araf) residue and wherein the O-acetyl group at position 2 of 3-D-Rhap is present in about 50% to about 100% of the repeating units. In an embodiment, said serotype 22A capsular saccharide comprises between about 80 to about 99.5 α-D-galactofuranose (α-D-Galf) residues and between about 0.5 to about 20 α-L-Arabinofuranose (α-L-Araf) residues in every 100 saccharide repeat units of the saccharide.

In an embodiment, said serotype 22A capsular saccharide comprises between about 90 to about 99.5 α-D-galactofuranose (α-D-Galf) residues and between about 0.5 to about 10 α-L-Arabinofuranose (α-L-Araf) residues in every 100 saccharide repeat units of the saccharide.

In an embodiment, said serotype 22A capsular saccharide comprises between about 95 to about 99.5 α-D-galactofuranose (α-D-Galf) residues and between about 0.5 to about 5 α-L-Arabinofuranose (α-L-Araf) residues in every 100 saccharide repeat units of the saccharide.

In an embodiment, said serotype 22A capsular saccharide comprises about 90 α-D-galactofuranose (α-D-Galf) residues and about 10 α-L-Arabinofuranose (α-L-Araf) residues in every 100 saccharide repeat units of the saccharide.

In an embodiment, said serotype 22A capsular saccharide comprises about 95 α-D-galactofuranose (α-D-Galf) residues and about 5 α-L-Arabinofuranose (α-L-Araf) residues in every 100 saccharide repeat units of the saccharide.

In an embodiment, said serotype 22A capsular saccharide comprises about 96 α-D-galactofuranose (α-D-Galf) residues and about 4 α-L-Arabinofuranose (α-L-Araf) residues in every 100 saccharide repeat units of the saccharide.

In an embodiment, said serotype 22A capsular saccharide comprises about 97 α-D-galactofuranose (α-D-Galf) residues and about 3 α-L-Arabinofuranose (α-L-Araf) residues in every 100 saccharide repeat units of the saccharide.

In an embodiment, said serotype 22A capsular saccharide comprises about 98 α-D-galactofuranose (α-D-Galf) residues and about 2 α-L-Arabinofuranose (α-L-Araf) residues in every 100 saccharide repeat units of the saccharide.

In an embodiment, said serotype 22A capsular saccharide comprises about 99 α-D-galactofuranose (α-D-Galf) residues and about 1 α-L-Arabinofuranose (α-L-Araf) residues in every 100 saccharide repeat units of the saccharide.

In an embodiment, said serotype 22A capsular saccharide comprises about 99.5 α-D-galactofuranose (α-D-Galf) residues and about 0.5 α-L-Arabinofuranose (α-L-Araf) residues in every 100 saccharide repeat units of the saccharide.

In an embodiment, the serotype 22A glycoconjugate of the present section 2.2 is prepared by direct reductive amination.

2.3 Mode of Preparation of the Streptococcus pneumoniae Serotype 22A Glycoconjugates of the Invention

The serotype 22A glycoconjugate of the present invention can be prepared by any coupling technique known to those of ordinary skill in the art.

In an embodiment, the serotype 22A saccharide is coupled to the carrier protein via non-covalent bonds (see e.g. WO2012155007, WO2020056202).

In an embodiment, the serotype 22A saccharide is conjugated via covalent bonds. In one embodiment the capsular saccharide is conjugated directly to a carrier protein. In a second embodiment the capsular saccharide is conjugated to a carrier protein through a spacer/linker.

In an embodiment, the serotype 22A glycoconjugate of the present invention is conjugated to the carrier protein via a linker, for instance a bifunctional linker. The linker is optionally heterobifunctional or homobifunctional, having for example a reactive amino group and a reactive carboxylic acid group, two reactive amino groups or two reactive carboxylic acid groups. The linker has for example between 4 and 20, 4 and 12, 5 and 10 carbon atoms.

A possible linker is adipic acid dihydrazide (ADH). Other linkers include B-propionamido (WO 00/10599), nitrophenyl-ethylamine (Gever et al (1979) Med. Microbiol. Immunol. 165; 171-288), haloalkyl halides (U.S. Pat. No. 4,057,685), glycosidic linkages (U.S. Pat. Nos. 4,673,574, 4,808,700), hexane diamine and 6-aminocaproic acid (U.S. Pat. No. 4,459,286).

In an embodiment, the serotype 22A glycoconjugate of the present invention is conjugated directly to the carrier protein (without a linker).

In general the following types of chemical groups on a protein carrier can be used for coupling/conjugation:
1) Amino group (for instance via lysine). In one embodiment this group is linked to carboxyl groups on saccharides directly or to a carboxyl group on a linker with carbodiimide chemistry e.g. with EDAC (1-Ethyl-3-(3-dimethylaminopropyl)carbodiimide). In another embodiment this group is linked to hydroxyl groups activated with CDAP or CNBr on saccharides directly or to such groups on a linker; to saccharides or linkers having an aldehyde group; to saccharides or linkers having a succinimide ester group.
2) Carboxyl (for instance via aspartic acid or glutamic acid). In one embodiment this group is linked to amino groups on saccharides directly or to an amino group on a linker with carbodiimide chemistry e.g. with EDAC.
3) Sulphydryl (for instance via cysteine). In one embodiment this group is linked to a bromo or chloro acetylated saccharide or linker with maleimide chemistry. In one embodiment this group is activated/modified with bis diazobenzidine.
4) Hydroxyl group (for instance via tyrosine). In one embodiment this group is activated/modified with bis diazobenzidine.
5) Imidazolyl group (for instance via histidine). In one embodiment this group is activated/modified with bis diazobenzidine.
6) Guanidyl group (for instance via arginine).
7) Indolyl group (for instance via tryptophan).

On the serotype 22A saccharide, in general the following groups can be used for a coupling: OH, COOH or NH2. Aldehyde groups can be generated after different treatments known in the art such as: periodate, acid hydrolysis, hydrogen peroxide, etc.

In an embodiment, the serotype 22A glycoconjugate of the present invention is prepared using CDAP chemistry. In said embodiment, the serotype 22A saccharide is activated with 1-cyano-4-dimethylamino pyridinium tetrafluoroborate (CDAP) to form a cyanate ester. The activated saccharide can then be coupled directly or via a spacer (linker) group to an amino group on the carrier protein. For example, the spacer could be cystamine or cysteamine to give a thiolated polysaccharide which can be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)).

In a preferred embodiment, the cyanate ester of the activated saccharide is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier. Such conjugates are described for example in WO 93/15760, WO 95/08348 and WO 96/129094.

In an embodiment, the serotype 22A glycoconjugate of the present invention is prepared using carbodiimides, hydrazides, active esters, norborane, p-nitrobenzoic acid, N-hydroxysuccinimide, S—NHS, EDC, TSTU. Many are described in International Patent Application Publication No. WO 98/42721. Conjugation may involve a carbonyl linker which may be formed by reaction of a free hydroxyl group of the saccharide with CDI (see Bethell et al. (1979) 1. Biol. Chern. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

Direct Reductive Amination In an embodiment, the serotype 22A glycoconjugate of the present invention is prepared by direct reductive amination (see e.g. U.S. Pat. Nos. 4,365,170, 4,673,574, WO2006/110381, WO2008/079653, WO2008/143709, WO2008/079732, WO2011/110531, WO2012/119972, WO2015110941, WO2015110940, WO2018/144439, WO2018/156491). According to the present invention, reductive amination involves two steps, (1) oxidation (activation) of the serotype 22A purified saccharide, (2) reduction of the activated saccharide and the carrier protein (e.g., CRM₁₉₇, TT or SCP) to form a glycoconjugate.

As mentioned above, before oxidation, sizing of the serotype 22A saccharide to a target molecular weight (MW) range can be performed. Therefore, in an embodiment, the isolated polysaccharide is sized before oxidation.

In an embodiment, the serotype 22A saccharide of the invention is conjugated to a carrier protein by a process comprising the step of:
(a) reacting said serotype 22A saccharide with an oxidizing agent;
(b) compounding the activated saccharide of step (a) with a carrier protein; and
(c) reacting the compounded activated saccharide and carrier protein with a reducing agent to form a glycoconjugate.

In an embodiment, the serotype 22A saccharide of the invention is conjugated to a carrier protein by a process comprising the step of:
(a) reacting said serotype 22A saccharide with an oxidizing agent;
(a′) quenching the oxidation reaction by addition of a quenching agent;
(b) compounding the activated saccharide of step (a′) with a carrier protein; and
(c) reacting the compounded activated saccharide and carrier protein with a reducing agent to form a glycoconjugate.

Following the oxidation step (a) the saccharide is said to be activated and is referred to as “activated saccharide”.

In an embodiment, the oxidizing agent is any oxidizing agent which oxidizes a terminal hydroxyl group to an aldehyde. In an embodiment, the oxidizing agent is periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate).

In an embodiment, the oxidizing agent is periodate in the presence of bivalent cations (see WO2008/143709).

In an embodiment, the oxidizing agent is periodic acid. In an embodiment, the oxidizing agent is periodic acid in the presence of bivalent cations. In an embodiment, the oxidizing agent is periodic acid in the presence of Mg²⁺. In an embodiment, the oxidizing agent is periodic acid in the presence of Ca²⁺. In an embodiment, the oxidizing agent is orthoperiodate.

In a preferred embodiment, the oxidizing agent is sodium periodate. In an embodiment, the periodate used for the oxidation is metaperiodate. In an embodiment the periodate used for the oxidation is sodium metaperiodate.

When a polysaccharide reacts with periodate, periodate oxidises vicinal hydroxyl groups to form carbonyl or aldehyde groups and causes cleavage of a C—C bond. For this reason, the term “reacting a polysaccharide with periodate” includes oxidation of vicinal hydroxyl groups by periodate.

In one embodiment step a) comprises reacting the polysaccharide with 0.01-2 molar equivalents of periodate. In one embodiment step a) comprises reacting the polysaccharide with 0.1-1.0 molar equivalents of periodate. In one embodiment step a) comprises reacting the polysaccharide with 0.1-0.5 molar equivalents of periodate.

In an embodiment, the oxidizing agent is a mixture of a stable nitroxyl radical compound with an oxidant (see WO2014097099).

In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO or 4-Amino-TEMPO, 4-Acetamido-2,2,6,6-tetramethylpiperidine 1-oxyl. Preferably said stable nitroxyl radical compound is TEMPO. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL.

In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In one embodiment, the quenching agent of step a′) is selected from vicinal diols, 1,2-aminoalcohols, amino acids, glutathione, sulfite, bisulfate, dithionite, metabisulfite, thiosulfate, phosphites, hypophosphites or phosphorous acid.

In one embodiment, the quenching agent is a 1,2-aminoalcohols of formula (I):

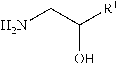

wherein R¹ is selected from H, methyl, ethyl, propyl or isopropyl.

In one embodiment, the quenching agent is selected from sodium and potassium salts of sulfite, bisulfate, dithionite, metabisulfite, thiosulfate, phosphites, hypophosphites or phosphorous acid.

In one embodiment, the quenching agent is an amino acid. In such embodiments, said amino acid may be selected from serine, threonine, cysteine, cystine, methionine, proline, hydroxyproline, tryptophan, tyrosine, and histidine.

In one embodiment, the quenching agent is a sulfite such as bisulfate, dithionite, metabisulfite, thiosulfate.

In one embodiment, the quenching agent is a compound comprising two vicinal hydroxyl groups (vicinal diols), i.e., two hydroxyl groups covalently linked to two adjacent carbon atoms. Preferably, the quenching agent is a compound of formula (II):

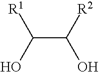

wherein R¹ and R² are each independently selected from H, methyl, ethyl, propyl or isopropyl.

In a preferred embodiment, the quenching agent is glycerol, ethylene glycol, propan-1,2-diol, butan-1,2-diol or butan-2,3-diol, or ascorbic acid. In an even preferred embodiment, the quenching agent is butan-2,3-diol.

In a preferred embodiment the degree of oxidation (also named “degree of activation” in the present document) of the activated serotype 22A saccharide is between 2 and 30. In an embodiment the degree of oxidation (DO) of the activated serotype 22A polysaccharide is between 10 and 25.

In one embodiment the activated saccharide and the carrier protein are lyophilised before step b).

In an embodiment the initial input ratio (weight by weight) of activated serotype 22A saccharide to carrier protein at step b) is between 4:1 and 0.1:1. In an embodiment the initial input ratio (weight by weight) of activated serotype 22A saccharide to carrier protein at step b) is between 1.5:1 and 0.5:1.

In an embodiment, the reduction reaction (c) is carried out in aqueous solvent. In another embodiment, the reduction reaction (c) is carried out in aprotic solvent.

In an embodiment, the reduction reaction (c) is carried out in the presence of dimethylsulphoxide (DMSO) or dimethylformamide (DMF). In an embodiment, the reduction reaction (c) is carried out in the presence of dimethylformamide (DMF). In an embodiment, the reduction reaction (c) is carried out in the presence of dimethylsulphoxide (DMSO).

In one embodiment the reduction reaction (c) is carried out in a solution consisting essentially of dimethylsulphoxide (DMSO) or dimethylformamide (DMF). In one embodiment the reduction reaction (c) is carried out in a solution consisting essentially of dimethylformamide (DMF). In one embodiment the reduction reaction (c) is carried out in a solution consisting essentially of dimethylsulphoxide (DMSO).

In an embodiment, the reduction reaction (c) is carried out in DMSO (dimethylsulfoxide) or in DMF (dimethylformamide)) solvent. In an embodiment, the reduction reaction (c) is carried out in DMSO (dimethylsulfoxide) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In an embodiment, the reducing agent is sodium triacetoxyborohydride. In a preferred embodiment, the reducing agent is sodium cyanoborohydride. In an embodiment, the reducing agent is sodium cyanoborohydride in the present of nickel (see WO2018144439).

In one embodiment between 0.2 and 20 molar equivalents of reducing agent is used at step c). In one embodiment between 0.5 and 10 molar equivalents of reducing agent is used at step c). In one embodiment between 1.0 and 5 molar equivalents of reducing agent is used at step c).

At the end of the reduction reaction, there may be unreacted aldehyde groups remaining in the conjugates, these may be capped using a suitable capping agent. In one embodiment this capping agent is sodium borohydride (NaBH₄). In an embodiment capping is achieved by mixing the product of step c) with 1 to 20 molar equivalents of sodium borohydride. In an embodiment capping is achieved by mixing the product of step c) with 1 to 10 molar equivalents of sodium borohydride. In an embodiment capping is achieved by mixing the product of step c) with 1 to 5 molar equivalents of sodium borohydride.

CDI and/or CDT Chemistry

In an embodiment, the serotype 22A glycoconjugate of the present invention is prepared by CDI and/or CDT chemistry as disclosed in WO2022249107.

CDI and/or CDT chemistry involves two steps, (1) reacting the serotype 22A saccharide with CDI and/or CDT in an aprotic solvent to produce an activated saccharide (activation), (2) reacting the activated saccharide with a carrier protein (e.g. CRM₁₉₇, TT or SCP) to form a glycoconjugate.

In an embodiment, the activating agent of step (1) is 1,1′-carbonyldiimidazole (CDI). In an embodiment, the activating agent of step (1) is 1,1′-Carbonyl-di-(1,2,4-triazole) (CDT).

As mentioned above, before activation with CDI and/or CDT, sizing of the serotype 22A saccharide to a target molecular weight (MW) range can be performed.

Therefore, in an embodiment, the serotype 22A saccharide is sized before activation with CDI. In an embodiment, the isolated polysaccharide is sized before activation with CDT. In an embodiment, the serotype 22A saccharide is sized to any of the target molecular weight (MW) range defined above.

Therefore, in an embodiment, the serotype 22A saccharide is conjugated to a carrier protein by a process comprising the step of:
(a) reacting said isolated polysaccharide with CDI and/or CDT in an aprotic solvent;
(b) reacting the activated polysaccharide of step (a) with a carrier protein in an aprotic solvent to form a glycoconjugate.

Following step (a) the polysaccharide is said to be activated and is referred to as “activated polysaccharide”.

In one embodiment step a) comprises reacting the serotype 22A saccharide with CDI.

In one embodiment step a) comprises reacting the serotype 22A saccharide with an amount of CDI that is between 0.5-10 molar equivalent to the amount of serotype 22A saccharide present in the reaction mixture.

In one embodiment step a) comprises reacting the serotype 22A saccharide with CDT.

In one embodiment step a) comprises reacting the serotype 22A saccharide with an amount of CDT hat is between 0.5-10 molar equivalent to the amount of serotype 22A saccharide present in the reaction mixture.

In an embodiment, the activating reaction a) is carried out in the presence of dimethylsulphoxide (DMSO), dimethylformamide (DMF), dimethylacetamide, N-methyl-2-pyrrolidone or hexamethylphosphoramide (HMPA). In an embodiment, the activating reaction a) is carried out in the presence of dimethylsulphoxide (DMSO).

In one embodiment the activating reaction a) is carried out in a solution consisting essentially of dimethylsulphoxide (DMSO) or dimethylformamide (DMF). In one embodiment the activating reaction a) is carried out in a solution consisting essentially of dimethylsulphoxide (DMSO).

In an embodiment, the conjugation reaction b) is carried out in the presence of dimethylsulphoxide (DMSO), dimethylformamide (DMF), dimethylacetamide, N-methyl-2-pyrrolidone or hexamethylphosphoramide (HMPA).

In an embodiment, the conjugation reaction b) is carried out in the presence of dimethylsulphoxide (DMSO).

In one embodiment the conjugation reaction b) is carried out in a solution consisting essentially of dimethylsulphoxide (DMSO) or dimethylformamide (DMF). In one embodiment the conjugation reaction b) is carried out in a solution consisting essentially of dimethylsulphoxide (DMSO).

In one embodiment, weak organic base can be added to the reaction mixture after the activating reaction a) but before the conjugation reaction b). The weak organic base can be added before or after the carrier protein is introduced the reaction mixture. Therefore, in one embodiment, the weak organic base is added to the reaction mixture before the carrier protein is introduced. In another embodiment, the weak organic base is added to the reaction mixture after the carrier protein is introduced. Weak organic base can be selected from alkanamines, imidazole, triazole, pyridine, histidine and guanidine. Alkanamines include alkyl primary amines such as methyl amine, ethylamine, propylamine, isopropylamine; alkyl secondary amines such as dimethyl amine, diethylamine, dipropylamine, diisopropylamine; alkyl tertially amines such as trimethyl amine, triethylamine, tri-isopropylamine, di-N,N′-isopropylethylamine, et al. In an embodiment, the weak organic base is an alkanamine. In an embodiment, the weak organic base is an imidazole. In an embodiment, the weak organic base is a triazole. In an embodiment, the weak organic base is pyridine. In an embodiment, the weak organic base is histidine. In an embodiment, the weak organic base is guanidine.

In one embodiment following the conjugation reaction b) unconjugated reactive sites of the activated polysaccharide are hydrolyzed. In one embodiment unconjugated reactive sites are hydrolyzed by addition to the conjugation solution of an aqueous solution. In one embodiment unconjugated reactive sites are hydrolyzed by addition to the conjugation solution of an aqueous buffered solution. In one embodiment unconjugated reactive sites are hydrolyzed by addition to the conjugation solution of an aqueous buffered solution and adjustment of the pH to between about 3.0 to about 10.0. In one embodiment unconjugated reactive sites are hydrolyzed by addition to the conjugation solution of an aqueous buffered solution and adjustment of the pH to between about 7.0 to about 10.0. In one embodiment unconjugated reactive sites are hydrolyzed by addition to the conjugation solution of an aqueous buffered solution and adjustment of the pH to between about 3.0 to about 7.0. In one embodiment unconjugated reactive sites are hydrolyzed by addition to the conjugation solution of an aqueous buffered solution and adjustment of the pH to about 4.0. In one embodiment unconjugated reactive sites are hydrolyzed by addition to the conjugation solution of an aqueous buffered solution and adjustment of the pH to about 9.0.

eTEC Chemistry

In an embodiment, the serotype 22A glycoconjugate of the present invention is prepared by eTEC chemistry as disclosed WO2014027302

The eTEC spacer includes seven linear atoms (i.e., —C(O)NH(CH2)2SCH2C(O)—) and provides stable thioether and amide bonds between the saccharide and carrier protein. Synthesis of the eTEC linked glycoconjugate involves reaction of an activated hydroxyl group of the saccharide with the amino group of a thioalkylamine reagent, e.g., cystamine or cysteinamine or a salt thereof, forming a carbamate linkage to the saccharide to provide a thiolated saccharide. Generation of one or more free sulfhydryl groups is accomplished by reaction with a reducing agent to provide an activated thiolated saccharide. Reaction of the free sulfhydryl groups of the activated thiolated saccharide with an activated carrier protein having one or more α-haloacetamide groups on amine containing residues generates a thioether bond to form the conjugate, wherein the carrier protein is attached to the eTEC spacer through an amide bond.

Therefore, in an embodiment, the serotype 22A glycoconjugate of the present invention comprises a serotype 22A saccharide covalently conjugated to a carrier protein through a (2-((2-oxoethyl)thio)ethyl)carbamate (eTEC) spacer.

In an embodiment, the serotype 22A glycoconjugate of the present invention comprises a serotype protein through a (2-((2-22A saccharide conjugated to a carrier oxoethyl)thio)ethyl)carbamate (eTEC) spacer, wherein the saccharide is covalently linked to the eTEC spacer through a carbamate linkage, and wherein the carrier protein is covalently linked to the eTEC spacer through an amide linkage.

The eTEC linked glycoconjugates of the invention may be represented by the general formula (III):

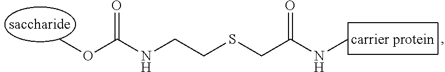

where (saccharide) represents the serotype 22A saccharide.

Formula (III) is a schematic representation of glycoconjugates of the invention. It should not be understood that only one linkage is present between the saccharide and the carrier protein. Rather, an individual carrier protein (CP) molecule may be linked to more than one serotype 22A saccharide molecule and an individual saccharide molecule can be linked to more than one individual carrier protein (CP) molecule. Additionally, a majority of the saccharide repeating unit remains unmodified and covalent linkages between the carrier protein and the saccharide is for a minority of the saccharide repeat units.

### Click Chemistry

In an embodiment, the serotype 22A glycoconjugate of the present invention is prepared by click chemistry (see e.g. PCT/IB2023/050202).

Therefore, in an embodiment, the serotype 22A glycoconjugate of the present invention comprises a serotype 22A saccharide covalently conjugated to a carrier protein (CP) through a spacer and having the general formula (IV):

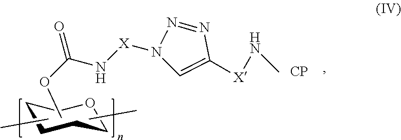

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 1 to 10 and m is selected from 1 to 4,
wherein X′ is selected from the group consisting of CH₂O(CH₂)_{n″}CH₂C═O, CH₂O(CH₂CH₂O) m′ (CH₂)_{n″}CH₂C═O, where n″ is selected from 0 to 10 and m′ is selected from 0 to 4,
wherein the structure in square backet represents a repeat unit of the serotype 22A saccharide and wherein n represents the number of repeating units.

In a particular aspect, the invention is directed to a serotype 22A glycoconjugate comprising a serotype 22A saccharide covalently conjugated to a carrier protein (CP) through a spacer and having the general formula (IV), wherein X is CH₂(CH₂)_{n′}, where n′ is 2 and wherein X′ is CH₂O(CH₂)_{n″}CH₂C═O where n″ is 1.

In a particular aspect, the invention pertains to a serotype 22A glycoconjugate comprising a serotype 22A saccharide covalently conjugated to a carrier protein (CP) through a spacer and having the general formula (V),

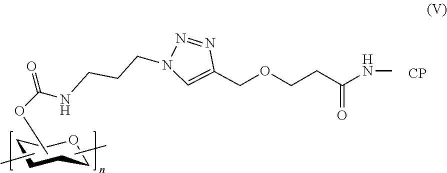

wherein the structure in square backet represents a repeat unit of the serotype 22A saccharide and wherein n represents the number of repeating units.

In an aspect, the serotype 22A glycoconjugate of the present invention comprises a serotype 22A saccharide covalently conjugated to a carrier protein (CP) through a spacer and having the general formula (VI):

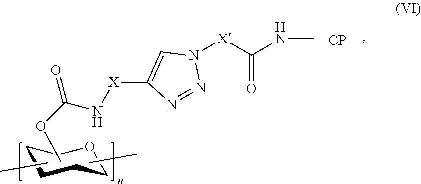

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 0 to 10 and m is selected from 1 to 4, wherein X′ is selected from the group consisting of CH₂(CH₂)_{n″}, CH₂O(CH₂)_{n″}CH₂, CH₂O(CH₂CH₂O)_{m′}(CH₂)_{n″}CH₂, where n″ is selected from 0 to 10 and m′ is selected from 0 to 4 and wherein the structure in square backet represents a repeat unit of the serotype 22A saccharide and wherein n represents the number of repeating units.

In a particular aspect, the invention is directed to a serotype 22A glycoconjugate comprising a serotype 22A saccharide covalently conjugated to a carrier protein (CP) through a spacer and having the general formula (VI), wherein X is CH₂(CH₂)_{n′}, where n′ is 0 and wherein X′ is CH₂(CH₂)_{n″} where n″ is 0.

In a particular aspect, the invention pertains to a serotype 22A glycoconjugate comprising a serotype 22A saccharide covalently conjugated to a carrier protein (CP) through a spacer and having the general formula (VII),

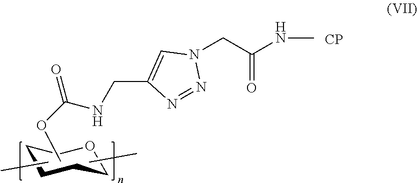

wherein the structure in square backet represents a repeat unit of the serotype 22A saccharide and wherein n represents the number of repeating units.

Formulas (IV), (V), (VII) and (VII) are schematic representations of glycoconjugates of the invention. It should not be understood that a linkage is present at every repeating unit of the saccharide (the structure in square brackets). Rather, a majority of the saccharide repeating unit remains unmodified and covalent linkages between the carrier protein and the saccharide is for a minority of the saccharide repeat units. Additionally, an individual carrier protein (CP) molecule may be linked to more than one saccharide molecule and an individual saccharide molecule can be linked to more than one individual carrier protein (CP) molecule. The structure in square brackets represents a repeat unit of the serotype 22A saccharide.

Following conjugation to the carrier protein, the serotype 22A glycoconjugate of the invention can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

### 2.4 Carrier Proteins

A component of the glycoconjugate is a carrier protein to which the serotype 22A saccharide is conjugated. The terms “protein carrier” or “carrier protein” or “carrier” may be used interchangeably herein. Carrier proteins should be amenable to standard conjugation procedures.

In a preferred embodiment, the carrier protein of the serotype 22A saccharide glycoconjugate is selected in the group consisting of: DT (Diphtheria Toxoid), TT (Tetanus Toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substution at glutamic acid 553 (Douglas et al. (1987) J. Bacteriol. 169 (11): 4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP). Another suitable carrier protein is rhizavidin [aa 45-179J-GGGGSSS-SP1500-AAA-SP0785] (CP1) (WO2020056202). Another suitable carrier protein is Rhavi-linker-PdT (G294P)-linker-SP0435 [aa 62-185] fusion protein (SPP2), see WO2023039223. WO2020/056202 and WO2023/039223 are incorporated by reference. SPP2 is described in particular at sections to of WO2023/039223.

In a preferred embodiment, the carrier protein of the serotype 22A saccharide glycoconjugate of the invention is selected from the group consisting of TT, DT, DT mutants (such as CRM₁₉₇), and a C5a peptidase from Streptococcus (SCP).

In an embodiment, the carrier protein of the serotype 22A saccharide glycoconjugate is DT (Diphtheria Toxoid). In another embodiment, the carrier protein of the serotype 22A saccharide glycoconjugate is TT (Tetanus Toxoid).

In another embodiment, the carrier protein of the serotype 22A saccharide glycoconjugate is PD (H. influenzae protein D; see, e.g., EP0594610 B).

In a preferred embodiment, the carrier protein of the serotype 22A saccharide glycoconjugate is CRM₁₉₇ or a C5a peptidase from Streptococcus (SCP).

In another embodiment, the carrier protein of the serotype 22A saccharide glycoconjugate is rhizavidin [aa 45-179J-GGGGSSS-SP1500-AAA-SP0785] (CP1).

In another embodiment, the carrier protein of the serotype 22A saccharide glycoconjugate is Rhavi-linker-PdT (G294P)-linker-SP0435 [aa 62-185] fusion protein (SPP2). In an embodiment, said SPP2 has the aminao acid sequence as set forth at SE ID NO: 19 of WO2023/039223.

In a preferred embodiment, the serotype 22A saccharide is conjugated to CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{lox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the serotype 22A saccharide is conjugated to CRM₁₉₇ protein. In an embodiment, the serotype 22A saccharide is conjugated to CRM₁₉₇ protein or the A chain of CRM₁₉₇ (see CN103495161). In an embodiment, the serotype 22A saccharide is conjugated the A chain of CRM₁₉₇ obtained via expression by genetically recombinant E. coli (see CN103495161).

In other preferred embodiments, the carrier protein of the serotype 22A saccharide glycoconjugate of the invention is SCP (Streptococcal C5a Peptidase).

Two important species of β-hemolytic streptococci, Streptococcus pyogenes (group A Streptococcus, GAS) and Streptococcus agalactiae (group B Streptococcus, GBS), which cause a variety of serious human infections that range from mild cases of pharyngitis and impetigo to serious invasive diseases such as necrotizing fasciitis (GAS) and neonatal sepsis (GBS) have developed a way to defeat this immune response. All human isolates of β-hemolytic streptococci, including GAS and GBS, produce a highly conserved cell-wall protein SCP (Streptococcal C5a Peptidase) that specifically inactivates C5a. The scp genes from GAS and GBS encode a polypeptide containing between 1,134 and 1,181 amino acids (Brown et al., PNAS, 2005, vol. 102, no. 51 pages 18391-18396). The first 31 residues are the export signal presequence and are removed upon passing through the cytoplasmic membrane. The next 68 residues serve as a pro-sequence and must be removed to produce active SCP. The next 10 residues can be removed without loss of protease activity. At the other end, starting with Lys-1034, are four consecutive 17-residue motifs followed by a cell sorting and cell-wall attachment signal. This combined signal is composed of a 20-residue hydrophilic sequence containing an LPTTND sequence, a 17-residue hydrophobic sequence, and a short basic carboxyl terminus.

SCP can be divided in domains (see FIG. 1B of Brown et al., PNAS, 2005, vol. 102, no. 51 pages 18391-18396). These domains are the Pre/Pro domain (which comprises the export signal presequence (commonly the first 31 residues) and the pro-sequence (commonly the next 68 residues)), the protease domain (which is splitted in two part (protease part 1 commonly residues 89-333/334 and protease domain part 2 and commonly residues 467/468-583/584), the protease-associated domain (PA domain) (commonly residues 333/334-467/468), three fibronectin type III (Fn) domains (Fn1, commonly residues 583/584-712/713; Fn2, commonly residues 712/713-928/929/930; commonly Fn3, residues 929/930-1029/1030/1031) and a cell wall anchor domain (commonly residues 1029/1030/1031 to the C-terminus).

In an embodiment, the carrier protein of the serotype 22A saccharide glycoconjugate of the invention is an SCP from GBS (SCPB). An example of SCPB is provided at SEQ. ID.NO: 3 of WO97/26008. See also SEQ ID NO: 3 of WO00/34487.

In another preferred embodiments, the carrier protein of the serotype 22A saccharide glycoconjugate of the invention is an SCP from GAS (SCPA).

Examples of SCPA can be found at SEQ.ID.No.1 and SEQ.ID.No.2 of WO97/26008. See also SEQ ID NO: 1, 2 and 23 of WO00/34487.

In a preferred embodiment, the carrier protein of the serotype 22A saccharide glycoconjugate of the invention is an enzymatically inactive SCP.

In other preferred embodiments, the carrier protein of the serotype 22A saccharide glycoconjugate of the invention is an enzymatically inactive SCP from GBS (SCPB).

In another preferred embodiments, the carrier protein of the serotype 22A saccharide glycoconjugate of the invention is an enzymatically inactive SCP from GAS (SCPA).

In an embodiment, the carrier protein of the serotype 22A saccharide glycoconjugate of the invention is a fragment of an SCP. In an embodiment, the carrier protein of the serotype 22A saccharide glycoconjugate of the invention is a fragment of an SCPA. Preferably, the carrier protein of the serotype 22A saccharide glycoconjugate of the invention is a fragment of an SCPB.

In an embodiment, the carrier protein of the serotype 22A saccharide glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 22A saccharide glycoconjugate of the invention is a fragment of an SCP which comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 22A saccharide glycoconjugate of the invention is an enzymatically inactive fragment of an SCP which comprises the protease domain, the protease-associated domain (PA domain) and two of the three fibronectin type III (Fn) domains but does not comprise the export signal presequence, the pro-sequence and the cell wall anchor domain.

In an embodiment, the carrier protein of the serotype 22A saccharide glycoconjugate of the invention is an enzymatically inactive fragment of an SCP. In an embodiment, said enzymatically inactive fragment of SCP comprises the protease domain, the protease-associated domain (PA domain) and the three fibronectin type III (Fn) domains but does not comprise the export signal presequence, the pro-sequence and the cell wall anchor domain.

In an embodiment, the carrier protein of the serotype 22A saccharide glycoconjugate of the invention is an enzymatically inactive fragment of an SCPA. In an embodiment, said enzymatically inactive fragment of an SCPA comprises the protease domain, the protease-associated domain (PA domain) and the three fibronectin type III (Fn) domains but does not comprise the export signal presequence, the pro-sequence and the cell wall anchor domain.

In a preferred embodiment, the carrier protein of the serotype 22A saccharide glycoconjugate of the invention is an enzymatically inactive fragment of SCPB. Preferably, said enzymatically inactive fragment of SCPB comprises the protease domain, the protease-associated domain (PA domain) and the three fibronectin type III (Fn) domains but does not comprise the export signal presequence, the pro-sequence and the cell wall anchor domain.

In an embodiment, the enzymatic activity of SCP is inactivated by replacing at least one amino acid of the wild type sequence. In an embodiment, said replacement is selected from the group consisting of D130A, H193A, N295A and S512A. The numbers indicate the amino acid residue position in the peptidase according to the numbering of SEQ ID NO: 1 of WO00/34487.

Therefore, in an embodiment, the carrier protein of the serotype 22A saccharide glycoconjugate of the invention is an enzymatically inactive SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 22A saccharide glycoconjugate of the invention is an enzymatically inactive SCPA where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 22A saccharide glycoconjugate of the invention is an enzymatically inactive SCPB where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 22A saccharide glycoconjugate of the invention is an enzymatically inactive fragment of an SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 22A saccharide glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 22A saccharide glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the serotype 22A saccharide glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the enzymatic activity of SCP is inactivated by replacing at least two amino acids of the wild type sequence. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In an embodiment, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

Therefore, in an embodiment, the carrier protein of the serotype 22A saccharide glycoconjugate of the invention is an enzymatically inactive SCP where said inactivation is accomplished by replacing at least two amino acids of the wild type sequence. Preferably, said replacement of at least two amino acids is in the protease domain. In an embodiment, said replacement of at least two amino acid is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 22A saccharide glycoconjugate of the invention is an enzymatically inactive SCPA where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 22A saccharide glycoconjugate of the invention is an enzymatically inactive SCPB where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 22A saccharide glycoconjugate of the invention is an enzymatically inactive fragment of an SCP where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 22A saccharide glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 22A saccharide glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least one amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the serotype 22A saccharide glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the enzymatic activity of SCP is inactivated by replacing at least three amino acids of the wild type sequence. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

Therefore, in an embodiment, the carrier protein of the serotype 22A saccharide glycoconjugate of the invention is an enzymatically inactive SCP where said inactivation is accomplished by replacing at least three amino acids of the wild type sequence. Preferably, said replacement of at least three amino acids is in the protease domain. In an embodiment, said replacement of at least three amino acid is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 22A saccharide glycoconjugate of the invention is an enzymatically inactive SCPA where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 22A saccharide glycoconjugate of the invention is an enzymatically inactive SCPB where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A.

In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A. In an embodiment, the carrier protein of the serotype 22A saccharide glycoconjugate of the invention is an enzymatically inactive fragment of an SCP where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 22A saccharide glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 22A saccharide glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the serotype 22A saccharide glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the enzymatic activity of SCP is inactivated by replacing at least four amino acids of the wild type sequence. In an embodiment, said at least four amino acids replacements are D130A, H193A, N295A and S512A.

Therefore, in an embodiment, the carrier protein of the serotype 22A saccharide glycoconjugate of the invention is an enzymatically inactive SCP where said inactivation is accomplished by replacing at least four amino acids of the wild type sequence. Preferably, said replacement of at least four amino acids is in the protease domain. In an embodiment, said replacement of at least four amino acid is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 22A saccharide glycoconjugate of the invention is an enzymatically inactive SCPA where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 22A saccharide glycoconjugate of the invention is an enzymatically inactive SCPB where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 22A saccharide glycoconjugate of the invention is an enzymatically inactive fragment of an SCP where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 22A saccharide glycoconjugate of the invention is an enzymatically inactive fragment of SCP which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the serotype 22A saccharide glycoconjugate of the invention is an enzymatically inactive fragment of SCPA which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least one amino acids is in part 2 of the protease domain. In an embodiment, said at least four amino acids replacements are D130A, H193A, N295A and S512A

In an embodiment, the carrier protein of the serotype 22A saccharide glycoconjugate of the invention is an enzymatically inactive fragment of SCPB which comprises the protease domain, the protease-associated domain (PA domain) and the three fibronectin type III (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acids is in part 2 of the protease domain. In an embodiment, said at least four amino acids replacements are D130A, H193A, N295A and S512A

In a particular embodiment, the carrier protein of the serotype 22A saccharide glycoconjugate of the invention is an enzymatically inactive fragment of SCP which consists of SEQ ID NO: 1.

In a particular embodiment, the carrier protein of the serotype 22A saccharide glycoconjugate of the invention is an enzymatically inactive fragment of SCP which consists of SEQ ID NO: 2.

SEQ ID NO: 1:
MAKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKVVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYVVEDMAGNITYTPVTKLLEGHSNKPEQ

SEQ ID NO: 1 is 950 amino acids long.

SEQ ID NO: 2:
AKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKVVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYVVEDMAGNITYTPVTKLLEGHSNKPEQ

SEQ ID NO: 2 is 949 amino acids long.

In a particular embodiment, the carrier protein of the serotype 22A saccharide glycoconjugate of the invention is an enzymatically inactive fragment of SCP consisting of a polypeptide having at least 90% identity with SEQ ID NO: 1.

In a particular embodiment, the carrier protein of the serotype 22A saccharide glycoconjugate of the invention is an enzymatically inactive fragment of SCP consisting of a polypeptide having at least 95% identity with SEQ ID NO: 1.

In a particular embodiment, the carrier protein of the serotype 22A saccharide glycoconjugate of the invention is an enzymatically inactive fragment of SCP consisting of a polypeptide having at least 99% identity with SEQ ID NO: 1.

In a particular embodiment, the carrier protein of the serotype 22A saccharide glycoconjugate of the invention is an enzymatically inactive fragment of SCP consisting of a polypeptide having at least 99.5% identity with SEQ ID NO: 1.

In a particular embodiment, the carrier protein of the serotype 22A saccharide glycoconjugate of the invention is an enzymatically inactive fragment of SCP consisting of a polypeptide having at least 99.8% identity with SEQ ID NO: 1.

In a particular embodiment, the carrier protein of the serotype 22A saccharide glycoconjugate of the invention is an enzymatically inactive fragment of SCP consisting of a polypeptide having at least 99.85% identity with SEQ ID NO: 1.

In a particular embodiment, the carrier protein of the serotype 22A saccharide glycoconjugate of the invention is an enzymatically inactive fragment of SCP consisting of a polypeptide having at least 90% identity with SEQ ID NO: 2.

In a particular embodiment, the carrier protein of the serotype 22A saccharide glycoconjugate of the invention is an enzymatically inactive fragment of SCP consisting of a polypeptide having at least 95% identity with SEQ ID NO: 2.

In a particular embodiment, the carrier protein of the serotype 22A saccharide glycoconjugate of the invention is an enzymatically inactive fragment of SCP consisting of a polypeptide having at least 99% identity with SEQ ID NO: 2.

In a particular embodiment, the carrier protein of the serotype 22A saccharide glycoconjugate of the invention is an enzymatically inactive fragment of SCP consisting of a polypeptide having at least 99.5% identity with SEQ ID NO: 2.

In a particular embodiment, the carrier protein of the serotype 22A saccharide glycoconjugate of the invention is an enzymatically inactive fragment of SCP consisting of a polypeptide having at least 99.8% identity with SEQ ID NO: 2.

In a particular embodiment, the carrier protein of the serotype 22A saccharide glycoconjugate of the invention is an enzymatically inactive fragment of SCP consisting of a polypeptide having at least 99.85% identity with SEQ ID NO: 2.

### 3. Immunogenic Compositions

In an embodiment the invention relates to an immunogenic composition comprising a S. pneumoniae serotype 22A saccharide of the invention.

In an embodiment the invention relates to an immunogenic composition comprising a Streptococcus pneumoniae serotype 22A glycoconjugate of the invention.

In an embodiment the invention relates to an immunogenic composition comprising a Streptococcus pneumoniae serotype 22A glycoconjugate of the invention and comprising from 1 to 45 different glycoconjugates.

In an embodiment the invention relates to an immunogenic composition comprising a Streptococcus pneumoniae serotype 22A glycoconjugate of the invention and comprising from 1 to 45 glycoconjugates from different serotypes of S. pneumoniae (1 to 45 pneumococcal conjugates).

In one embodiment the invention relates to an immunogenic composition comprising glycoconjugates from 7, 8, 9, 10, 11, 12, 13, 14, 15, 16, 17, 18, 19, 20, 21, 22, 23, 24 or 25 different serotypes of S. pneumoniae. In one embodiment the immunogenic composition comprises glycoconjugates from 16 or 20 different serotypes of S. pneumoniae.

In an embodiment the immunogenic composition is a 21, 22, 23, 24 or 25-valent pneumococcal conjugate composition.

In an embodiment the immunogenic composition is a 21-valent pneumococcal conjugate composition. In an embodiment the immunogenic composition is a 22-valent pneumococcal conjugate composition. In an embodiment the immunogenic composition is a 23-valent pneumococcal conjugate composition. In an embodiment the immunogenic composition is a 24-valent pneumococcal conjugate composition. In an embodiment the immunogenic composition is a 25-valent pneumococcal conjugate composition.

In an embodiment the invention relates to an immunogenic composition comprising a Streptococcus pneumoniae serotype 22A glycoconjugate of the invention and comprising from 26 to 45 glycoconjugates from different serotypes of S. pneumoniae (26 to 45 pneumococcal conjugates). In one embodiment the invention relates to an immunogenic composition comprising glycoconjugates from 26, 27, 28, 29, 30, 31, 32, 33, 34, 35, 36, 37, 38, 39, 40, 41, 42, 43, 44 or 45 different serotypes of S. pneumoniae.

In one embodiment the immunogenic composition comprises glycoconjugates from 35 or 45 different serotypes of S. pneumoniae. In an embodiment the immunogenic composition is a 35, 36, 37, 38, 39, 40, 41, 42, 43, 44 or 45-valent pneumococcal conjugate compositions. In an embodiment the immunogenic composition is a 40, 41, 42, 43, 44 or 45-valent pneumococcal conjugate compositions. In an embodiment the immunogenic composition is a 40-valent pneumococcal conjugate composition. In an embodiment the immunogenic composition is a 41-valent pneumococcal conjugate composition. In an embodiment the immunogenic composition is a 42-valent pneumococcal conjugate composition. In an embodiment the immunogenic composition is a 43-valent pneumococcal conjugate composition. In an embodiment the immunogenic composition is a 44-valent pneumococcal conjugate composition. In an embodiment the immunogenic composition is a 45-valent pneumococcal conjugate composition.

In an embodiment the invention relates to an immunogenic composition comprising a Streptococcus pneumoniae serotype 22A glycoconjugate of the invention and further comprising glycoconjugates from S. pneumoniae serotypes 4, 6B, 9V, 14, 18C, 19F and 23F.

In an embodiment said immunogenic composition comprises in addition glycoconjugates from S. pneumoniae serotypes 1, 5 and 7F.

In an embodiment any of the immunogenic compositions above comprises in addition glycoconjugates from S. pneumoniae serotype 3.

In an embodiment any of the immunogenic compositions above comprises in addition glycoconjugates from S. pneumoniae serotypes 6A and 19A.

In an embodiment any of the immunogenic compositions above comprise in addition a glycoconjugates from S. pneumoniae serotype 22F and 33F.

In an embodiment any of the immunogenic compositions above comprise in addition a glycoconjugates from S. pneumoniae serotypes 8, 10A, 11A, 12F and 15B.

In an embodiment any of the immunogenic compositions above comprise in addition a glycoconjugates from S. pneumoniae serotype 2.

In an embodiment any of the immunogenic compositions above comprise in addition a glycoconjugates from S. pneumoniae serotype 9N.

In an embodiment any of the immunogenic compositions above comprise in addition a glycoconjugates from S. pneumoniae serotype 17F.

In an embodiment any of the immunogenic compositions above comprise in addition a glycoconjugates from S. pneumoniae serotype 20.

In an embodiment any of the immunogenic compositions above comprise in addition a glycoconjugates from S. pneumoniae serotype 15C.

In an embodiment the invention relates to an immunogenic composition comprising a Streptococcus pneumoniae serotype 22A glycoconjugate of the invention and further comprising glycoconjugates from S. pneumoniae serotypes 4, 6B, 9V, 14, 18C, 19F and 23F. In an embodiment the immunogenic composition is an 8-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 4, 5, 6B, 7F, 9V, 14, 18C, 19F and 23F. In an embodiment the immunogenic composition is an 11-valent pneumococcal conjugate compositions.

In an embodiment the invention relates to an immunogenic composition comprising a S. pneumoniae serotype 22A glycoconjugate of the invention and further comprising glycoconjugates from S. pneumoniae serotypes 1, 3, 4, 5, 6A, 6B, 7F, 9V, 14, 18C, 19A, 19F and 23F. In an embodiment the immunogenic composition is a 14-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 9V, 14, 18C, 19A, 19F, 22F, 23F and 33F. In an embodiment the immunogenic composition is a 16-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F. In an embodiment the immunogenic composition is a 21-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15C, 18C, 19A, 19F, 22F, 23F and 33F. In an embodiment the immunogenic composition is a 21-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 2, 3, 4, 5, 6A, 6B, 7F, 8, 9V, 10A, 11A, 12F, 14, 15B, 18C, 19A, 19F, 22F, 23F and 33F. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 2, 3, 4, 5, 6A, 6B, 7F, 8, 9V, 10A, 11A, 12F, 14, 15C, 18C, 19A, 19F, 22F, 23F and 33F. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 9N, 10A, 11A, 12F, 14, 15B, 18C, 19A, 19F, 22F, 23F and 33F. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 2, 3, 4, 5, 6A, 6B, 7F, 8, 9V, 9N, 10A, 11A, 12F, 14, 15B, 18C, 19A, 19F, 22F, 23F and 33F. In an embodiment the immunogenic composition is a 23-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 2, 3, 4, 5, 6A, 6B, 7F, 8, 9V, 9N, 10A, 11A, 12F, 14, 15B, 17F, 18C, 19A, 19F, 22F, 23F and 33F. In an embodiment the immunogenic composition is a 24-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 2, 3, 4, 5, 6A, 6B, 7F, 8, 9V, 9N, 10A, 11A, 12F, 14, 15B, 17F, 18C, 19A, 19F, 20, 22F, 23F and 33F. In an embodiment the immunogenic composition is a 25-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F and 33F. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F and 33F. In an embodiment the immunogenic composition is a 23-valent pneumococcal conjugate compositions.

In an embodiment the invention relates to an immunogenic composition comprising a Streptococcus pneumoniae serotype 22A glycoconjugate of the invention and further comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In one embodiment, the S. pneumoniae saccharides are conjugated to CRM₁₉₇. In one embodiment, the S. pneumoniae saccharides from serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F, 35B and 22A are conjugated to CRM₁₉₇ and the S. pneumoniae saccharide from serotype 3 is conjugated to SCP. In an embodiment the immunogenic composition is a 26-valent pneumococcal conjugate compositions.

In an embodiment the invention relates to an immunogenic composition comprising a Streptococcus pneumoniae serotype 22A glycoconjugate of the invention and further comprising at least one glycoconjugate selected from the group consisting of glycoconjugates from S. pneumoniae serotypes 2, 3, 7C, 9N, 10B, 15A, 16F, 17F, 20, 21, 23A, 23B, 24B, 24F, 27, 29, 31, 33B, 34, 35B, 35F and 38.

In an embodiment the invention relates to an immunogenic composition comprising a Streptococcus pneumoniae serotype 22A glycoconjugate of the invention and further comprising twenty one glycoconjugates selected from the group consisting of glycoconjugates from S. pneumoniae serotypes 2, 3, 7C, 9N, 10B, 15A, 16F, 17F, 20, 21, 23A, 23B, 24B, 24F, 27, 29, 31, 33B, 34, 35B, 35F and 38. In an embodiment the immunogenic composition is a 22-valent pneumococcal conjugate compositions.

In an embodiment the invention relates to an immunogenic composition comprising a Streptococcus pneumoniae serotype 22A glycoconjugate of the invention and further comprising glycoconjugates from serotypes 2, 3, 7C, 9N, 10B, 15A, 16F, 17F, 20, 21, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a Streptococcus pneumoniae serotype 22A glycoconjugate of the invention and further comprising at least one glycoconjugate selected from the group consisting of glycoconjugates from S. pneumoniae serotypes 2, 3, 7C, 9N, 10B, 15A, 16F, 17F, 19A, 19F, 20, 21, 23A, 23B, 24B, 24F, 27, 29, 31, 33B, 34, 35B, 35F and 38.

In an embodiment the invention relates to an immunogenic composition comprising a Streptococcus pneumoniae serotype 22A glycoconjugate of the invention and further comprising twenty two glycoconjugates selected from the group consisting of glycoconjugates from S. pneumoniae serotypes 2, 3, 7C, 9N, 10B, 15A, 16F, 17F, 19A, 19F, 20, 21, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a Streptococcus pneumoniae serotype 22A glycoconjugate of the invention and further comprising twenty three glycoconjugates selected from the group consisting of glycoconjugates from S. pneumoniae serotypes 2, 3, 7C, 9N, 10B, 15A, 16F, 17F, 19A, 19F, 20, 21, 23A, 23B, 24B, 24F, 27, 29, 31, 33B, 34, 35B, 35F and 38. In an embodiment the immunogenic composition is a 24-valent pneumococcal conjugate compositions.

In an embodiment the invention relates to an immunogenic composition comprising a Streptococcus pneumoniae serotype 22A glycoconjugate of the invention and further comprising glycoconjugates from serotypes 2, 3, 7C, 9N, 10B, 15A, 16F, 17F, 20, 21, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a Streptococcus pneumoniae serotype 22A glycoconjugate of the invention and further comprising glycoconjugates from serotypes 2, 3, 7C, 9N, 10B, 15A, 16F, 17F, 19A, 19F, 20, 21, 23A, 23B, 24B, 24F, 27, 29, 31, 33B, 34, 35B, 35F and 38. In an embodiment the immunogenic composition is a 25-valent pneumococcal conjugate compositions.

Compositions of the invention may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

### 4 Adjuvant(s)

In some embodiments, the immunogenic compositions disclosed herein may further comprise at least one, two or three adjuvants. In some embodiments, the immunogenic compositions disclosed herein may further comprise at least one adjuvant.

In some embodiments, the immunogenic compositions disclosed herein may further comprise one adjuvant.

In some embodiments, the immunogenic compositions disclosed herein may further comprise two adjuvants.

The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide).

In a preferred embodiment, the immunogenic compositions disclosed herein comprise aluminum phosphate or aluminum hydroxide as adjuvant. In an even preferred embodiment, the immunogenic compositions disclosed herein comprise aluminum phosphate as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

In an embodiment of the present invention, the immunogenic compositions as disclosed herein comprise any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

### 5 Formulation

The immunogenic compositions of the invention may be formulated in liquid form (i.e., solutions or suspensions) or in a lyophilized form. In an embodiment, the immunogenic composition of the invention is formulated in a liquid form. In an embodiment, the immunogenic composition of the invention is formulated in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In some embodiments, the buffer is succinate. In some embodiments, the buffer is histidine. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer.

In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0.

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL. In an embodiment, the immunogenic composition of the invention for injection has a volume of 0.2 mL to 1 mL, even more preferably a volume of about 0.5 mL.

### 6 Uses of the Glycoconjugate and Immunogenic Compositions of the Invention

The glycoconjugates disclosed herein may be use as antigens. For example, they may be part of a vaccine.

Therefore, in an embodiment, the immunogenic compositions of the invention are for use as a medicament.

In an embodiment, the immunogenic compositions of the invention are for use as a vaccine.

Therefore, in an embodiment, the immunogenic compositions described herein are for use in generating an immune response in a subject. In one aspect, the subject is a mammal, such as a human, non-human primate, cat, sheep, pig, horse, bovine or dog. Preferably, the subject is a human.

The immunogenic compositions described herein may be used in therapeutic or prophylactic methods for preventing, treating or ameliorating a bacterial infection, disease or condition in a subject. In particular, immunogenic compositions described herein may be used to prevent, treat or ameliorate a S. pneumoniae infection, disease or condition in a subject. Preferably, the immunogenic compositions described herein may be used to prevent, treat or ameliorate S. pneumoniae infection, disease or condition by the serotypes contained in the composition in a subject.

Thus, in one aspect, the disclosure provides a method of preventing, treating or ameliorating an infection, disease or condition associated with S. pneumoniae serotype 22A in a subject, comprising administering to the subject an immunologically effective amount of an immunogenic composition of the disclosure.

In some such embodiments, the infection, disease or condition is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess.

In an embodiment, the disclosure provides a method of inducing an immune response to S. pneumoniae serotype 22A in a subject comprising administering to the subject an immunologically effective amount of an immunogenic composition of the invention. In one aspect, the subject is a mammal, such as a human, cat, sheep, pig, horse, bovine or dog. Preferably the subject is a human.

In an embodiment, the immunogenic compositions disclosed herein are for use as a vaccine. In such embodiments the immunogenic compositions described herein may be used to prevent S. pneumoniae serotype 22A infection in a subject. Thus, in one aspect, the invention provides a method of preventing an infection by S. pneumoniae serotype 22A in a subject comprising administering to the subject an immunologically effective amount of an immunogenic composition of the disclosure. In some such embodiments, the infection is selected from the group consisting of pneumonia, sinusitis, otitis media, acute otitis media, meningitis, bacteremia, sepsis, pleural empyema, conjunctivitis, osteomyelitis, septic arthritis, endocarditis, peritonitis, pericarditis, mastoiditis, cellulitis, soft tissue infection and brain abscess. In one aspect, the subject is a mammal, such as a human, cat, sheep, pig, horse, bovine or dog. Preferably the subject is a human.

The immunogenic composition of the present disclosure can be used to protect or treat a human susceptible to a S. pneumoniae serotype 22A infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

7 Subject to be Treated with the Immunogenic Compositions of the Invention

As disclosed herein, the immunogenic compositions described herein may be used in various therapeutic or prophylactic methods for preventing, treating or ameliorating a bacterial infection, disease or condition in a subject.

In a preferred embodiment, said subject is a human. In a most preferred embodiment, said subject is a newborn (i.e., under three months of age), an infant (i.e., from 3 months to one year of age) or a toddler (i.e., from one year to four years of age). In an embodiment, the immunogenic compositions disclosed herein are for use as a vaccine.

In such embodiment, the subject to be vaccinated may be less than 1 year of age. For example, the subject to be vaccinated can be about 1, about 2, about 3, about 4, about 5, about 6, about 7, about 8, about 9, about 10, about 11 or about 12 months of age. In an embodiment, the subject to be vaccinated is about 2, about 4 or about 6 months of age. In another embodiment, the subject to be vaccinated is less than 2 years of age. For example, the subject to be vaccinated can be about 12 to about 15 months of age. In some cases, as little as one dose of the immunogenic composition according to the invention is needed, but under some circumstances, a second, third or fourth dose may be given (see section 8 below).

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

In an embodiment of the present invention, the immunocompromised subject to be vaccinated suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is selected from the one disclosed on page 24, line 11, to page 25, line 19, of WO 2010/125480.

In a particular embodiment of the present invention, the immunocompromised subject to be vaccinated suffers from a disease selected from the groups consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised subject to be vaccinated suffers from malnutrition.

In a particular embodiment of the present invention, the immunocompromised subject to be vaccinated is taking a drug or treatment that lowers the body's resistance to infection. In an embodiment, said drug is selected from the one disclosed on page 26, line 33, to page 26, line 4, of WO 2010/125480.

In a particular embodiment of the present invention, the immunocompromised subject to be vaccinated is a smoker.

In a particular embodiment of the present invention, the immunocompromised subject to be vaccinated has a white blood cell count (leukocyte count) below 5×10⁹ cells per liter, or below 4×10⁹ cells per liter, or below 3×10⁹ cells per liter, or below 2×10⁹ cells per liter, or below 1×10° cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised subject to be vaccinated suffers from neutropenia. In a particular embodiment of the present invention, the immunocompromised subject to be vaccinated has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter.

A low white blood cell count or “neutropenia” is a condition characterized by abnormally low levels of neutrophils in the circulating blood. Neutrophils are a specific kind of white blood cell that help to prevent and fight infections. The most common reason that cancer patients experience neutropenia is as a side effect of chemotherapy. Chemotherapy-induced neutropenia increases a patient's risk of infection and disrupts cancer treatment.

In a particular embodiment of the present invention, the immunocompromised subject to be vaccinated has a CD4+ cell count below 500/mm³, or CD4+ cell count below 300/mm³, or CD4+ cell count below 200/mm³, CD4+ cell count below 100/mm³, CD4+ cell count below 75/mm³, or CD4+ cell count below 50/mm³.

CD4 cell tests are normally reported as the number of cells in mm³. Normal CD4 counts are between 500 and 1,600, and CD8 counts are between 375 and 1,100. CD4 counts drop dramatically in people with HIV.

In an embodiment of the invention, any of the immunocompromised subjects disclosed herein is a human male or a human female.

### 8 Regimen

In some cases, as little as one dose of the immunogenic composition according to the invention is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the invention is a single dose. In a particular embodiment, said single dose schedule is for healthy persons being at least 2 years of age.

In an embodiment, the schedule of vaccination of the immunogenic composition according to the invention is a multiple dose schedule. In a particular embodiment, said multiple dose schedule consists of a series of 2 doses separated by an interval of about 1 month to about 2 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses separated by an interval of about 1 month to about 2 months. In another embodiment, said multiple dose schedule consists of a series of 3 doses separated by an interval of about 1 month, or a series of 3 doses separated by an interval of about 2 months.

In another embodiment, said multiple dose schedule consists of a series of 3 doses separated by an interval of about 1 month to about 2 months followed by a fourth dose about 10 months to about 13 months after the first dose. In another embodiment, said multiple dose schedule consists of a series of 3 doses separated by an interval of about 1 month followed by a fourth dose about 10 months to about 13 months after the first dose, or a series of 3 doses separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses separated by an interval of about 1 month to about 2 months (for example 28-56 days between doses), starting at 2 months of age, and followed by a toddler dose at 12-18 months of age. In an embodiment, said multiple dose schedule consists of a series of 3 doses separated by an interval of about 1 month to about 2 months (for example 28-56 days between doses), starting at 2 months of age, and followed by a toddler dose at 12-15 months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more boosts are given at intervals that range from about 2 to about 24 weeks, preferably with a dosing interval of 4-8 weeks.

In an embodiment, a prime dose is given at day 0 and a boost is given about 3 months later.

### 9. Analytical Methods

Native serotype 22A polysaccharide is O-acetylated and the total amount of O-acetylation has been found to be approximately one O-acetyl group per polysaccharide repeating unit. The degree of O-acetylation of the polysaccharide can be determined by any method known in the art, for example, by proton NMR or by ion-HPLC analysis (see for example Lemercinier et al. (1996) Carbohydrate Research 296:83-96; Jones et al. (2002) J. Pharmaceutical and Biomedical Analysis 30:1233-1247; Hestrin, S. (1949) J. Biol. Chem. 180:249-261).

In an embodiment the invention relates to a method of detecting the presence of O-acetyl groups in an isolated S. pneumoniae serotype 22A polysaccharide, said method comprising the step of: a) isolating an S. pneumoniae serotype 22A polysaccharide and b) detecting the presence of O-acetyl groups in said polysaccharide.

In an embodiment the presence of O-acetyl groups is detected by NMR, Mass Spectrometry (MS) or High-performance liquid chromatography (HPLC).

In an embodiment the presence of O-acetyl groups is detected by NMR.

In an embodiment the presence of O-acetyl groups is detected by Mass Spectrometry (MS).

In a preferred embodiment the presence of O-acetyl groups is detected by High-performance liquid chromatography (HPLC).

In an embodiment, the invention relates to a method of detecting the presence of O-acetyl groups in an oxidized serotype 22A polysaccharide, said method comprising the step of: a) reacting an isolated S. pneumoniae serotype 22A polysaccharide with an oxidizing agent and b) detecting the presence of O-acetyl groups in said oxidized polysaccharide.

In an embodiment the presence of O-acetyl groups is detected by NMR, Mass Spectrometry (MS) or High-performance liquid chromatography (HPLC).

In an embodiment the presence of O-acetyl groups is detected by NMR.

In an embodiment the presence of O-acetyl groups is detected by Mass Spectrometry (MS).

In a preferred embodiment the presence of O-acetyl groups is detected by High-performance liquid chromatography (HPLC).

In an embodiment, said oxidizing agent is any oxidizing agent which oxidizes a terminal hydroxyl group to an aldehyde. In an embodiment, the oxidizing agent is periodate. In an embodiment, the oxidizing agent is orthoperiodate. In a preferred embodiment, the oxidizing agent is sodium periodate. In an embodiment, the oxidizing agent is metaperiodate. In a preferred embodiment the oxidizing agent is sodium metaperiodate.

In an embodiment, said oxidizing agent is a stable nitroxyl radical compound and an oxidant. In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO or 4-Acetamido-2,2,6,6-tetramethylpiperidine 1-oxyl. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL. In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In an embodiment the invention relates to a method of detecting the presence of O-acetyl groups in S. pneumoniae serotype 22A glycoconjugate, said method comprising the step of: a) preparing a S. pneumoniae serotype 22A glycoconjugate and b) detecting the presence of O-acetyl groups in said glycoconjugate.

In an embodiment the presence of O-acetyl groups is detected by NMR, Mass Spectrometry (MS) or High-performance liquid chromatography (HPLC).

In an embodiment the presence of O-acetyl groups is detected by NMR.

In an embodiment the presence of O-acetyl groups is detected by Mass Spectrometry (MS).

In a preferred embodiment the presence of O-acetyl groups is detected by High-performance liquid chromatography (HPLC).

In an embodiment the invention relates to a method of measuring the degree of O-acetylation of an isolated S. pneumoniae serotype 22A polysaccharide, said method comprising the step of: a) isolating an S. pneumoniae serotype 22A polysaccharide and b) measuring the amount of O-acetyl groups in said polysaccharide.

In an embodiment the amount of O-acetyl groups is measured by NMR, Mass Spectrometry (MS) or High-performance liquid chromatography (HPLC).

In an embodiment the amount of O-acetyl groups is measured by NMR.

In an embodiment the amount of O-acetyl groups is measured by Mass Spectrometry (MS).

In a preferred embodiment the amount of O-acetyl groups is measured by High-performance liquid chromatography (HPLC).

In an embodiment the invention relates to a method of measuring the degree of O-acetylation of an isolated S. pneumoniae serotype 22A polysaccharide, said method comprising the step of: a) isolating an S. pneumoniae serotype 22A polysaccharide b) treating said polysaccharide with a base and c) measuring the amount of O-acetyl groups in said polysaccharide.

In an embodiment the amount of O-acetyl groups is measured by NMR, Mass Spectrometry (MS) or High-performance liquid chromatography (HPLC).

In an embodiment the amount of O-acetyl groups is measured by NMR.

In an embodiment the amount of O-acetyl groups is measured by Mass Spectrometry (MS).

In a preferred embodiment the amount of O-acetyl groups is measured by High-performance liquid chromatography (HPLC).

In an embodiment, the invention relates to a method of measuring the degree of O-acetylation in an oxidized serotype 22A polysaccharide, said method comprising the step of: a) reacting an isolated S. pneumoniae serotype 22A polysaccharide with an oxidizing agent and b) measuring the amount of O-acetyl groups in said oxidized polysaccharide.

In an embodiment the amount of O-acetyl groups is measured by NMR, Mass Spectrometry (MS) or High-performance liquid chromatography (HPLC).

In an embodiment the amount of O-acetyl groups is measured by NMR.

In an embodiment the amount of O-acetyl groups is measured by Mass Spectrometry (MS).

In a preferred embodiment the amount of O-acetyl groups is measured by High-performance liquid chromatography (HPLC).

In an embodiment, said oxidizing agent is any oxidizing agent which oxidizes a terminal hydroxyl group to an aldehyde. In an embodiment, the oxidizing agent is periodate. In an embodiment, the oxidizing agent is orthoperiodate. In a preferred embodiment, the oxidizing agent is sodium periodate. In an embodiment, the oxidizing agent is metaperiodate. In a preferred embodiment the oxidizing agent is sodium metaperiodate.

In an embodiment, said oxidizing agent is a stable nitroxyl radical compound and an oxidant. In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO or 4-Acetamido-2,2,6,6-tetramethylpiperidine 1-oxyl. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL. In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In an embodiment the invention relates to a method of measuring the degree of O-acetylation in S. pneumoniae serotype 22A glycoconjugate, said method comprising the step of: a) preparing a S. pneumoniae serotype 22A glycoconjugate and b) measuring the amount of O-acetyl groups in said glycoconjugate.

In an embodiment the amount of O-acetyl groups is measured by NMR, Mass Spectrometry (MS) or High-performance liquid chromatography (HPLC).

In an embodiment the amount of O-acetyl groups is measured by NMR.

In an embodiment the amount of O-acetyl groups is measured by Mass Spectrometry (MS).

In a preferred embodiment the amount of O-acetyl groups is measured by High-performance liquid chromatography (HPLC).

In an embodiment, the invention relates to a method of detecting the presence of L-Arabinofuranose-5-Aldehyde residues in an oxidized serotype 22A polysaccharide, said method comprising the step of: a) reacting an isolated S. pneumoniae serotype 22A polysaccharide with an oxidizing agent and b) detecting the presence of L-Arabinofuranose-5-Aldehyde residues in said oxidized polysaccharide.

In an embodiment the presence of L-Arabinofuranose-5-Aldehyde residues is detected by NMR. In an embodiment the presence of L-Arabinofuranose-5-Aldehyde residues is detected by 2D NMR.

In a preferred embodiment, the presence of L-Arabinofuranose-5-Aldehyde residues is detected by 2D ¹H-¹³C HSQC NMR.

In an embodiment, the presence of L-Arabinofuranose-5-Aldehyde residues is detected by Mass Spectrometry (MS). In an embodiment, the presence of L-Arabinofuranose-5-Aldehyde residues is detected by Tandem Mass Spectrometry (MS/MS). In an embodiment, the presence of L-Arabinofuranose-5-Aldehyde residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment, the presence of L-Arabinofuranose-5-Aldehyde residues is detected by Size-Exclusion Chromatography combined with Mass Spectrometry (SEC/MS).

In an embodiment, the presence of L-Arabinofuranose-5-Aldehyde residues is detected by Gas Chromatography-Mass Spectrometry (GC-MS). In an embodiment, the presence of L-Arabinofuranose-5-Aldehyde residues is detected by Liquid Chromatography-Mass Spectrometry (LC-MS). In an embodiment, the presence of L-Arabinofuranose-5-Aldehyde residues is detected by Capillary Electrophoresis-Mass Spectrometry (CE-MS). In an embodiment, the presence of L-Arabinofuranose-5-Aldehyde residues is detected by Ion Mobility Spectrometry-Mass Spectrometry (IMS/MS). In an embodiment, the presence of L-Arabinofuranose-5-Aldehyde residues is detected by Hydrophilic Interaction Liquid Chromatography-Mass Spectrometry (HILIC-LC/MS).

In an embodiment, said oxidizing agent is any oxidizing agent which oxidizes a terminal hydroxyl group to an aldehyde. In an embodiment, the oxidizing agent is periodate. In an embodiment, the oxidizing agent is orthoperiodate. In a preferred embodiment, the oxidizing agent is sodium periodate. In an embodiment, the oxidizing agent is metaperiodate. In a preferred embodiment the oxidizing agent is sodium metaperiodate.

In an embodiment, said oxidizing agent is a stable nitroxyl radical compound and an oxidant. In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO or 4-Acetamido-2,2,6,6-tetramethylpiperidine 1-oxyl. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL. In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide. In an aspect, said stable nitroxyl radical compound is 2,2,6,6-Tetramethyl-1-piperidinyloxy free radical (TEMPO) and said oxidant is N-Chlorosuccinimide (NCS).

In an embodiment, the invention relates to a method of detecting the presence of α-L-Arabinofuranose (α-L-Araf) residues in a reduced serotype 22A polysaccharide, said method comprising the step of: a) reacting an isolated S. pneumoniae serotype 22A polysaccharide with a reducing agent and b) detecting the presence of α-L-Arabinofuranose (α-L-Araf) residues in said reduced polysaccharide.

In an embodiment the presence of α-L-Arabinofuranose (α-L-Araf) residues is detected by NMR. In an embodiment the presence of α-L-Arabinofuranose (α-L-Araf) residues is detected by 2D NMR.

In an embodiment the presence of α-L-Arabinofuranose (α-L-Araf) residues is detected by Heteronuclear Single Quantum Coherence Spectroscopy (HSQC), Heteronuclear multiple-bond correlation spectroscopy (HMBC), Correlation spectroscopy (COSY), and/or Heteronuclear Single Quantum Coherence Spectroscopy-Total Correlation Spectroscopy (HSQC-TOCSY). In a preferred embodiment, the presence of α-L-Arabinofuranose (α-L-Araf) residues is detected by 2D ¹H-¹³C HSQC NMR.

In an embodiment the presence of α-L-Arabinofuranose (α-L-Araf) residues is detected by Mass Spectrometry (MS). In an embodiment the presence of α-L-Arabinofuranose (α-L-Araf) residues is detected by Tandem Mass Spectrometry (MS/MS). In an embodiment the presence of α-L-Arabinofuranose (α-L-Araf) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of α-L-Arabinofuranose (α-L-Araf) residues is detected by Size-Exclusion Chromatography combined with Mass Spectrometry (SEC/MS).

In an embodiment the presence of α-L-Arabinofuranose (α-L-Araf) residues is detected by Gas Chromatography-Mass Spectrometry (GC-MS). In an embodiment the presence of α-L-Arabinofuranose (α-L-Araf) residues is detected by Liquid Chromatography-Mass Spectrometry (LC-MS). In an embodiment the presence of α-L-Arabinofuranose (α-L-Araf) residues is detected by Capillary Electrophoresis-Mass Spectrometry (CE-MS). In an embodiment the presence of α-L-Arabinofuranose (α-L-Araf) residues is detected by Ion Mobility Spectrometry-Mass Spectrometry (IMS/MS). In an embodiment the presence of α-L-Arabinofuranose (α-L-Araf) residues is detected by Hydrophilic Interaction Liquid Chromatography-Mass Spectrometry (HILIC-LC/MS).

In an embodiment, said reducing agent is sodium borohydride (NaBH₄).

In an embodiment, said isolated S. pneumoniae serotype 22A polysaccharide has been previously treated with an oxidizing agent. In an embodiment, the oxidizing agent is any oxidizing agent which oxidizes a terminal hydroxyl group to an aldehyde. In an embodiment, the oxidizing agent is periodate. In an embodiment, the oxidizing agent is orthoperiodate. In a preferred embodiment, the oxidizing agent is sodium periodate. In an embodiment, the oxidizing agent is metaperiodate. In a preferred embodiment the oxidizing agent is sodium metaperiodate.

In an embodiment, said isolated S. pneumoniae serotype 22A polysaccharide has been previously treated with a stable nitroxyl radical compound and an oxidant. In an aspect, said stable nitroxyl radical compound is a molecule bearing a TEMPO or a PROXYL (2,2,5,5-tetramethyl-1-pyrrolidinyloxy) moiety. Preferably said molecule has the ability to selectively oxidize primary alcohol in the presence of an oxidant, to generate aldehyde groups, without affecting secondary hydroxyl groups. More preferably said molecule has the ability to selectively oxidize primary alcohol in the presence of an oxidant, to generate aldehyde groups, without over oxidation to carboxyl groups. In an aspect, said stable nitroxyl radical compound is TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO or 4-Acetamido-2,2,6,6-tetramethylpiperidine 1-oxyl. In an aspect, said stable nitroxyl radical compound is selected from the groups consisting of TEMPO, 2,2,6,6-Tetramethyl-4-(methylsulfonyloxy)-1-piperidinooxy, 4-Phosphonooxy-TEMPO, 4-Oxo-TEMPO, 4-Methoxy-TEMPO, 4-Isothiocyanato-TEMPO, 4-(2-Iodoacetamido)-TEMPO free radical, 4-Hydroxy-TEMPO, 4-Cyano-TEMPO, 4-Carboxy-TEMPO, 4-(2-Bromoacetamido)-TEMPO, 4-Amino-TEMPO, 4-Acetamido-2,2,6,6-tetramethylpiperidine 1-oxyl. Preferably said stable nitroxyl radical compound is TEMPO. In a further aspect, said stable nitroxyl radical compound is 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL or 3-Cyano-PROXYL. In a further aspect, said stable nitroxyl radical compound is selected from the groups consisting of 3β-DOXYL-5α-cholestane, 5-DOXYL-stearic acid, 16-DOXYL-stearic acid, Methyl 5-DOXYL-stearate, 3-(Aminomethyl)-PROXYL, 3-Carbamoyl-PROXYL, 3-Carbamoyl-2,2,5,5-tetramethyl-3-pyrrolin-1-oxyl, 3-Carboxy-PROXYL, 3-Cyano-PROXYL. In an aspect, the oxidant is a molecule bearing a N-halo moiety. Preferably said molecule has the ability to selectively oxidize primary alcohol in the presence of a nitroxyl radical compound. In an aspect, said oxidant is N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid or 1,3,5-triiodo-1,3,5-triazinane-2,4,6-trione. In an aspect, said oxidant is selected from the group consisting of N-Chlorosuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

In an aspect, said stable nitroxyl radical compound is 2,2,6,6-Tetramethyl-1-piperidinyloxy free radical (TEMPO) and said oxidant is N-Chlorosuccinimide (NCS).

In an embodiment the invention relates to a method of detecting the presence of α-L-Arabinofuranose (α-L-Araf) residues in S. pneumoniae serotype 22A glycoconjugate, said method comprising the step of: a) preparing a S. pneumoniae serotype 22A glycoconjugate and b) detecting the presence of α-L-Arabinofuranose (α-L-Araf) residues in said glycoconjugate.

In an embodiment the presence of α-L-Arabinofuranose (α-L-Araf) residues is detected by NMR. In an embodiment the presence of α-L-Arabinofuranose (α-L-Araf) residues is detected by 2D NMR.

In an embodiment the presence of α-L-Arabinofuranose (α-L-Araf) residues is detected by Heteronuclear Single Quantum Coherence Spectroscopy (HSQC), Heteronuclear multiple-bond correlation spectroscopy (HMBC), Correlation spectroscopy (COSY), and/or Heteronuclear Single Quantum Coherence Spectroscopy-Total Correlation Spectroscopy (HSQC-TOCSY). In a preferred embodiment, the presence of α-L-Arabinofuranose (α-L-Araf) residues is detected by 2D ¹H-¹³C HSQC NMR.

In an embodiment the presence of α-L-Arabinofuranose (α-L-Araf) residues is detected by Mass Spectrometry (MS). In an embodiment the presence of α-L-Arabinofuranose (α-L-Araf) residues is detected by Tandem Mass Spectrometry (MS/MS). In an embodiment the presence of α-L-Arabinofuranose (α-L-Araf) residues is detected by Gas Chromatography-Mass Spectrometry (GC-MS), Liquid Chromatography-Mass Spectrometry (LC-MS), Capillary Electrophoresis-Mass Spectrometry (CE-MS) or Ion Mobility Spectrometry-Mass Spectrometry (IMS/MS or IMMS). In an embodiment the presence of α-L-Arabinofuranose (α-L-Araf) residues is detected by Size-Exclusion Chromatography combined with Mass Spectrometry (SEC/MS).

In an embodiment the presence of N α-L-Arabinofuranose (α-L-Araf) residues is detected by Gas Chromatography-Mass Spectrometry (GC-MS). In an embodiment the presence of α-L-Arabinofuranose (α-L-Araf) residues is detected by Liquid Chromatography-Mass Spectrometry (LC-MS). In an embodiment the presence of N α-L-Arabinofuranose (α-L-Araf) residues is detected by Capillary Electrophoresis-Mass Spectrometry (CE-MS). In an embodiment the presence of α-L-Arabinofuranose (α-L-Araf) residues is detected by Ion Mobility Spectrometry-Mass Spectrometry (IMS/MS). In an embodiment the presence of α-L-Arabinofuranose (α-L-Araf) residues is detected by Hydrophilic Interaction Liquid Chromatography-Mass Spectrometry (HILIC-LC/MS).

### 10. Particular Embodiments of the Invention are Set Forth in the Following Numbered Paragraphs 1 to 321:

1. An isolated S. pneumoniae serotype 22A saccharide with the following repeating unit:

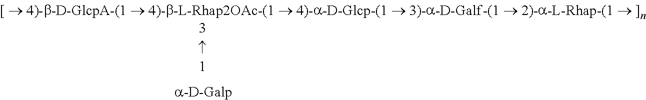

where n represents the number of repeating units.

2. An isolated S. pneumoniae serotype 22A saccharide with the following repeating unit:

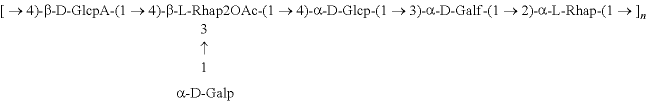

where n represents the number of repeating units and wherein the O-acetyl group at position 2 of β-D-Rhap is present in about 100% of the repeating units.

3. An isolated S. pneumoniae serotype 22A saccharide with the following repeating unit:

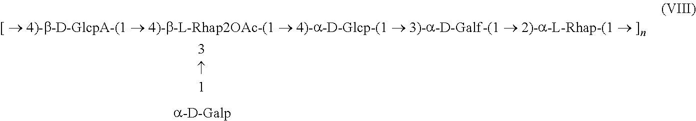

where n represents the number of repeating units and wherein the O-acetyl group at position 2 of β-D-Rhap is present in about 50% to about 100% of the repeating units.

4. The isolated S. pneumoniae serotype 22A saccharide of paragraph 3 wherein the O-acetyl group at position 2 of 3-D-Rhap is present in about 60% to about 95% of the repeating units.

5. The isolated S. pneumoniae serotype 22A saccharide of paragraph 3 wherein the O-acetyl group at position 2 of 3-D-Rhap is present in about 80% to about 99% of the repeating units.

6. The isolated S. pneumoniae serotype 22A saccharide of paragraph 3 wherein the O-acetyl group at position 2 of 3-D-Rhap is present in about 0% to about 95% of the repeating units.

7. The isolated S. pneumoniae serotype 22A saccharide of paragraph 3 wherein the O-acetyl group at position 2 of β-D-Rhap is present in about 10% to about 90% of the repeating units.

8. The isolated S. pneumoniae serotype 22A saccharide of paragraph 3 wherein the O-acetyl group at position 2 of 3-D-Rhap is present in about 50% to about 80% of the repeating units.

9. An isolated S. pneumoniae serotype 22A saccharide with the following repeating unit:

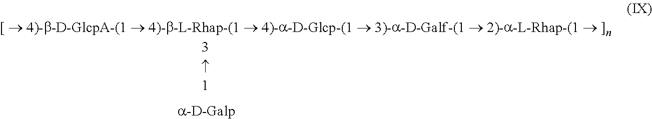

where n represents the number of repeating units.

10. The isolated S. pneumoniae serotype 22A saccharide of any one of paragraphs 1-9 having between 10 and 5,000 repeating units.

11. The isolated S. pneumoniae serotype 22A saccharide of any one of paragraphs 1-9 having between 50 and 4,500 repeating units.

12. The isolated S. pneumoniae serotype 22A saccharide of any one of paragraphs 1-9 having between 100 and 4,500 repeating units.

13. The isolated S. pneumoniae serotype 22A saccharide of any one of paragraphs 1-9 having between 150 and 2,000 repeating units.

14. The isolated S. pneumoniae serotype 22A saccharide of any one of paragraphs 1-9 having a weight average molecular weight between 5 kDa and 5000 kDa.

15. The isolated S. pneumoniae serotype 22A saccharide of any one of paragraphs 1-9 having a weight average molecular weight between 5 kDa and 2000 kDa.

16. The isolated S. pneumoniae serotype 22A saccharide of any one of paragraphs 1-9 having a weight average molecular weight between 50 kDa and 5000 kDa.

17. The isolated S. pneumoniae serotype 22A saccharide of any one of paragraphs 1-9 having a weight average molecular weight between 50 kDa and 1000 kDa.

18. The isolated S. pneumoniae serotype 22A saccharide of any one of paragraphs 1-9 having a weight average molecular weight between 100 kDa and 5000 kDa.

19. The isolated S. pneumoniae serotype 22A saccharide of any one of paragraphs 1-9 having a weight average molecular weight between 100 kDa and 1000 kDa.

20. The isolated S. pneumoniae serotype 22A saccharide of any one of paragraphs 1-9 having a weight average molecular weight between 100 kDa and 500 kDa.

21. The isolated S. pneumoniae serotype 22A saccharide of any one of paragraphs 1-9 having a weight average molecular weight between 300 kDa and 5000 kDa.

22. The isolated S. pneumoniae serotype 22A saccharide of any one of paragraphs 1-9 having a weight average molecular weight between 300 kDa and 1000 kDa.

23. The isolated S. pneumoniae serotype 22A saccharide of any one of paragraphs 1-9 having a weight average molecular weight between 500 kDa and 3000 kDa.

24. The isolated S. pneumoniae serotype 22A saccharide of any one of paragraphs 1-9 having a weight average molecular weight between 500 kDa and 2000 kDa.

25. The isolated S. pneumoniae serotype 22A saccharide of any one of paragraphs 1-9 having a weight average molecular weight between 500 kDa and 1000 kDa.

26. The isolated S. pneumoniae serotype 22A saccharide of any one of paragraphs 1-9 sized to a weight average molecular weight between 10 kDa and 1000 kDa.

27. The isolated S. pneumoniae serotype 22A saccharide of any one of paragraphs 1-9 sized to a weight average molecular weight between 50 kDa and 500 kDa.

28. The isolated S. pneumoniae serotype 22A saccharide of any one of paragraphs 1-9 sized to a weight average molecular weight between 100 kDa and 400 kDa.

29. A glycoconjugate comprising an isolated S. pneumoniae serotype 22A saccharide of any one of paragraphs 1-28 conjugated to a carrier protein.

30. A glycoconjugate consisting of an isolated S. pneumoniae serotype 22A saccharide of any one of paragraphs 1-28 conjugated to a carrier protein.

31. A serotype 22A glycoconjugate comprising a serotype 22A capsular saccharide comprising between about 1 to about 70 α-L-Arabinofuranose (α-L-Araf) residues in every 100 saccharide repeat units of the saccharide.

32. A serotype 22A glycoconjugate comprising a serotype 22A capsular saccharide comprising between about 1 to about 60 α-L-Arabinofuranose (α-L-Araf) residues in every 100 saccharide repeat units of the saccharide.

33. A serotype 22A glycoconjugate comprising a serotype 22A capsular saccharide comprising between about 1 to about 50 α-L-Arabinofuranose (α-L-Araf) residues in every 100 saccharide repeat units of the saccharide.

34. A serotype 22A glycoconjugate comprising a serotype 22A capsular saccharide comprising between about 1 to about 40 α-L-Arabinofuranose (α-L-Araf) residues in every 100 saccharide repeat units of the saccharide.

35. A serotype 22A glycoconjugate comprising a serotype 22A capsular saccharide comprising between about 1 to about 30 α-L-Arabinofuranose (α-L-Araf) residues in every 100 saccharide repeat units of the saccharide.

36. A serotype 22A glycoconjugate comprising a serotype 22A capsular saccharide comprising between about 1 to about 20 α-L-Arabinofuranose (α-L-Araf) residues in every 100 saccharide repeat units of the saccharide.

37. A serotype 22A glycoconjugate comprising a serotype 22A capsular saccharide comprising between about 1 to about 10 α-L-Arabinofuranose (α-L-Araf) residues in every 100 saccharide repeat units of the saccharide.

38. A serotype 22A glycoconjugate comprising a serotype 22A capsular saccharide comprising between about 1 to about 5 α-L-Arabinofuranose (α-L-Araf) residues in every 100 saccharide repeat units of the saccharide.

39. A serotype 22A glycoconjugate comprising a serotype 22A capsular saccharide comprising between about 5 to about 70 α-L-Arabinofuranose (α-L-Araf) residues in every 100 saccharide repeat units of the saccharide.

40. A serotype 22A glycoconjugate comprising a serotype 22A capsular saccharide comprising between about 5 to about 60 α-L-Arabinofuranose (α-L-Araf) residues in every 100 saccharide repeat units of the saccharide.

41. A serotype 22A glycoconjugate comprising a serotype 22A capsular saccharide comprising between about 5 to about 50 α-L-Arabinofuranose (α-L-Araf) residues in every 100 saccharide repeat units of the saccharide.

42. A serotype 22A glycoconjugate comprising a serotype 22A capsular saccharide comprising between about 5 to about 40 α-L-Arabinofuranose (α-L-Araf) residues in every 100 saccharide repeat units of the saccharide.

43. A serotype 22A glycoconjugate comprising a serotype 22A capsular saccharide comprising between about 5 to about 30 α-L-Arabinofuranose (α-L-Araf) residues in every 100 saccharide repeat units of the saccharide.

44. A serotype 22A glycoconjugate comprising a serotype 22A capsular saccharide comprising between about 5 to about 20 α-L-Arabinofuranose (α-L-Araf) residues in every 100 saccharide repeat units of the saccharide.

45. A serotype 22A glycoconjugate comprising a serotype 22A capsular saccharide comprising between about 5 to about 10 α-L-Arabinofuranose (α-L-Araf) residues in every 100 saccharide repeat units of the saccharide.

46. A serotype 22A glycoconjugate comprising a serotype 22A capsular saccharide comprising between about 10 to about 70 α-L-Arabinofuranose (α-L-Araf) residues in every 100 saccharide repeat units of the saccharide.

47. A serotype 22A glycoconjugate comprising a serotype 22A capsular saccharide comprising between about 10 to about 60 α-L-Arabinofuranose (α-L-Araf) residues in every 100 saccharide repeat units of the saccharide.

48. A serotype 22A glycoconjugate comprising a serotype 22A capsular saccharide comprising between about 10 to about 50 α-L-Arabinofuranose (α-L-Araf) residues in every 100 saccharide repeat units of the saccharide.

49. A serotype 22A glycoconjugate comprising a serotype 22A capsular saccharide comprising between about 10 to about 40 α-L-Arabinofuranose (α-L-Araf) residues in every 100 saccharide repeat units of the saccharide.

50. A serotype 22A glycoconjugate comprising a serotype 22A capsular saccharide comprising between about 10 to about 30 α-L-Arabinofuranose (α-L-Araf) residues in every 100 saccharide repeat units of the saccharide.

51. A serotype 22A glycoconjugate comprising a serotype 22A capsular saccharide comprising between about 10 to about 20 α-L-Arabinofuranose (α-L-Araf) residues in every 100 saccharide repeat units of the saccharide.

52. A serotype 22A glycoconjugate comprising a serotype 22A capsular saccharide comprising about 1 α-L-Arabinofuranose (α-L-Araf) residue in every 100 saccharide repeat units of the saccharide.

53. A serotype 22A glycoconjugate comprising a serotype 22A capsular saccharide comprising about 2 α-L-Arabinofuranose (α-L-Araf) residues in every 100 saccharide repeat units of the saccharide.

54. A serotype 22A glycoconjugate comprising a serotype 22A capsular saccharide comprising about 3 α-L-Arabinofuranose (α-L-Araf) residues in every 100 saccharide repeat units of the saccharide.

55. A serotype 22A glycoconjugate comprising a serotype 22A capsular saccharide comprising about 4 α-L-Arabinofuranose (α-L-Araf) residues in every 100 saccharide repeat units of the saccharide.

56. A serotype 22A glycoconjugate comprising a serotype 22A capsular saccharide comprising about 5 α-L-Arabinofuranose (α-L-Araf) residues in every 100 saccharide repeat units of the saccharide.

57. A serotype 22A glycoconjugate comprising a serotype 22A capsular saccharide comprising about 10 α-L-Arabinofuranose (α-L-Araf) residues in every 100 saccharide repeat units of the saccharide.

58. A serotype 22A glycoconjugate comprising a serotype 22A capsular saccharide comprising about 20 α-L-Arabinofuranose (α-L-Araf) residues in every 100 saccharide repeat units of the saccharide.

59. A serotype 22A glycoconjugate comprising a serotype 22A capsular saccharide with the following repeating unit:

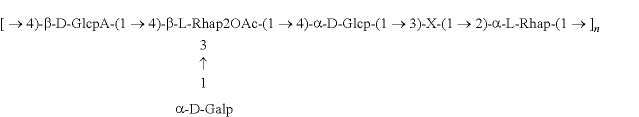

where n represents the number of repeating units, wherein X represents either a α-D-galactofuranose (α-D-Galf) or a α-L-Arabinofuranose (α-L-Araf) residue.

60. The serotype 22A glycoconjugate of paragraph 59, wherein said serotype 22A capsular saccharide comprises between about 70 to about 99.5 α-D-galactofuranose (α-D-Galf) residues and between about 0.5 to about 30 α-L-Arabinofuranose (α-L-Araf) residues in every 100 saccharide repeat units of the saccharide.

61. The serotype 22A glycoconjugate of paragraph 59, wherein said serotype 22A capsular saccharide comprises between about 80 to about 99.5 α-D-galactofuranose (α-D-Galf) residues and between about 0.5 to about 20 α-L-Arabinofuranose (α-L-Araf) residues in every 100 saccharide repeat units of the saccharide.

62. The serotype 22A glycoconjugate of paragraph 59, wherein said serotype 22A capsular saccharide comprises between about 90 to about 99.5 α-D-galactofuranose (α-D-Galf) residues and between about 0.5 to about 10 α-L-Arabinofuranose (α-L-Araf) residues in every 100 saccharide repeat units of the saccharide.

63. The serotype 22A glycoconjugate of paragraph 59, wherein said serotype 22A capsular saccharide comprises between about 95 to about 99.5 α-D-galactofuranose (α-D-Galf) residues and between about 0.5 to about 5 α-L-Arabinofuranose (α-L-Araf) residues in every 100 saccharide repeat units of the saccharide.

64. The serotype 22A glycoconjugate of paragraph 59, wherein said serotype 22A capsular saccharide comprises about 90 α-D-galactofuranose (α-D-Galf) residues and about 10 α-L-Arabinofuranose (α-L-Araf) residues in every 100 saccharide repeat units of the saccharide.

65. The serotype 22A glycoconjugate of paragraph 59, wherein said serotype 22A capsular saccharide comprises about 95 α-D-galactofuranose (α-D-Galf) residues and about 5 α-L-Arabinofuranose (α-L-Araf) residues in every 100 saccharide repeat units of the saccharide.

66. The serotype 22A glycoconjugate of paragraph 59, wherein said serotype 22A capsular saccharide comprises about 96 α-D-galactofuranose (α-D-Galf) residues and about 4 α-L-Arabinofuranose (α-L-Araf) residues in every 100 saccharide repeat units of the saccharide.

67. The serotype 22A glycoconjugate of paragraph 59, wherein said serotype 22A capsular saccharide comprises about 97 α-D-galactofuranose (α-D-Galf) residues and about 3 α-L-Arabinofuranose (α-L-Araf) residues in every 100 saccharide repeat units of the saccharide.

68. The serotype 22A glycoconjugate of paragraph 59, wherein said serotype 22A capsular saccharide comprises about 98 α-D-galactofuranose (α-D-Galf) residues and about 2 α-L-Arabinofuranose (α-L-Araf) residues in every 100 saccharide repeat units of the saccharide.

69. The serotype 22A glycoconjugate of paragraph 59, wherein said serotype 22A capsular saccharide comprises about 99 α-D-galactofuranose (α-D-Galf) residues and about 1 α-L-Arabinofuranose (α-L-Araf) residues in every 100 saccharide repeat units of the saccharide.

70. The serotype 22A glycoconjugate of paragraph 59, wherein said serotype 22A capsular saccharide comprises about 99.5 α-D-galactofuranose (α-D-Galf) residues and about 0.5 α-L-Arabinofuranose (α-L-Araf) residues in every 100 saccharide repeat units of the saccharide.

71. A serotype 22A glycoconjugate comprising a serotype 22A capsular saccharide with the following repeating unit:

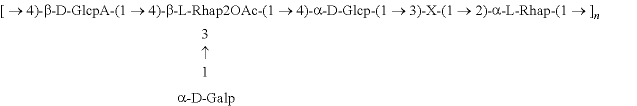

where n represents the number of repeating units, wherein X represents either a α-D-galactofuranose (α-D-Galf) or a α-L-Arabinofuranose (α-L-Araf) residue and wherein the O-acetyl group at position 2 of β-D-Rhap is present in about 50% to about 100% of the repeating units. In an embodiment, said serotype 22A capsular saccharide comprises between about 80 to about 99.5 α-D-galactofuranose (α-D-Galf) residues and between about 0.5 to about 20 α-L-Arabinofuranose (α-L-Araf) residues in every 100 saccharide repeat units of the saccharide.

72. The serotype 22A glycoconjugate of paragraph 71, wherein said serotype 22A capsular saccharide comprises between about 90 to about 99.5 α-D-galactofuranose (α-D-Galf) residues and between about 0.5 to about 10 α-L-Arabinofuranose (α-L-Araf) residues in every 100 saccharide repeat units of the saccharide.

73. The serotype 22A glycoconjugate of paragraph 71, wherein said serotype 22A capsular saccharide comprises between about 95 to about 99.5 α-D-galactofuranose (α-D-Galf) residues and between about 0.5 to about 5 α-L-Arabinofuranose (α-L-Araf) residues in every 100 saccharide repeat units of the saccharide.

74. The serotype 22A glycoconjugate of paragraph 71, wherein said serotype 22A capsular saccharide comprises about 90 α-D-galactofuranose (α-D-Galf) residues and about 10 α-L-Arabinofuranose (α-L-Araf) residues in every 100 saccharide repeat units of the saccharide.

75. The serotype 22A glycoconjugate of paragraph 71, wherein said serotype 22A capsular saccharide comprises about 95 α-D-galactofuranose (α-D-Galf) residues and about 5 α-L-Arabinofuranose (α-L-Araf) residues in every 100 saccharide repeat units of the saccharide.

76. The serotype 22A glycoconjugate of paragraph 71, wherein said serotype 22A capsular saccharide comprises about 96 α-D-galactofuranose (α-D-Galf) residues and about 4 α-L-Arabinofuranose (α-L-Araf) residues in every 100 saccharide repeat units of the saccharide.

77. The serotype 22A glycoconjugate of paragraph 71, wherein said serotype 22A capsular saccharide comprises about 97 α-D-galactofuranose (α-D-Galf) residues and about 3 α-L-Arabinofuranose (α-L-Araf) residues in every 100 saccharide repeat units of the saccharide.

78. The serotype 22A glycoconjugate of paragraph 71, wherein said serotype 22A capsular saccharide comprises about 98 α-D-galactofuranose (α-D-Galf) residues and about 2 α-L-Arabinofuranose (α-L-Araf) residues in every 100 saccharide repeat units of the saccharide.

79. The serotype 22A glycoconjugate of paragraph 71, wherein said serotype 22A capsular saccharide comprises about 99 α-D-galactofuranose (α-D-Galf) residues and about 1 α-L-Arabinofuranose (α-L-Araf) residues in every 100 saccharide repeat units of the saccharide.

80. The serotype 22A glycoconjugate of paragraph 71, wherein said serotype 22A capsular saccharide comprises about 99.5 α-D-galactofuranose (α-D-Galf) residues and about 0.5 α-L-Arabinofuranose (α-L-Araf) residues in every 100 saccharide repeat units of the saccharide.

81. The serotype 22A glycoconjugate of any one of paragraphs 31-80 wherein said serotype 22A glycoconjugate is prepared by direct reductive amination.

81a. The serotype 22A glycoconjugate of paragraph 29 or 30 wherein said serotype 22A glycoconjugate comprises a serotype 22A saccharide covalently conjugated to a carrier protein through a (2-((2-oxoethyl)thio)ethyl)carbamate (eTEC) spacer.

82. The serotype 22A glycoconjugate of paragraph 29 or 30 wherein said serotype 22A glycoconjugate is prepared by click chemistry.

83. A serotype 22A glycoconjugate comprising a serotype 22A saccharide covalently conjugated to a carrier protein (CP) through a spacer and having the general formula (IV):

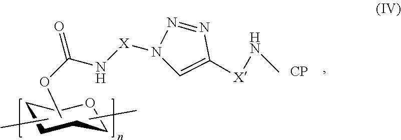

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 1 to 10 and m is selected from 1 to 4,
wherein X′ is selected from the group consisting of CH₂O(CH₂)_{n″}CH₂C═O, CH₂O(CH₂CH₂O) m′ (CH₂)_{n″}CH₂C═O, where n″ is selected from 0 to 10 and m′ is selected from 0 to 4, wherein the structure in square backet represents a repeat unit of the serotype 22A saccharide and wherein n represents the number of repeating units.

84. A serotype 22A glycoconjugate comprising a serotype 22A saccharide covalently conjugated to a carrier protein (CP) through a spacer and having the general formula (IV), wherein X is CH₂(CH₂)_{n′}, where n′ is 2 and wherein X′ is CH₂O(CH₂)_{n″}CH₂C═O where n″ is 1.

85. A serotype 22A glycoconjugate comprising a serotype 22A saccharide covalently conjugated to a carrier protein (CP) through a spacer and having the general formula (V),

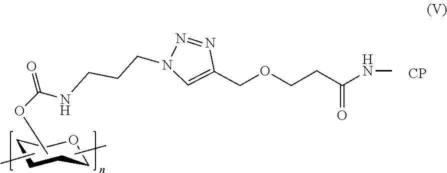

wherein the structure in square backet represents a repeat unit of the serotype 22A saccharide and wherein n represents the number of repeating units.

86. A serotype 22A glycoconjugate comprising a serotype 22A saccharide covalently conjugated to a carrier protein (CP) through a spacer and having the general formula (VI):

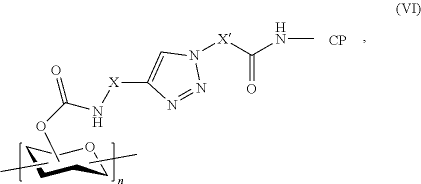

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 0 to 10 and m is selected from 1 to 4,
wherein X′ is selected from the group consisting of CH₂(CH₂)_{n″}, CH₂O(CH₂)_{n″}CH₂, CH₂O(CH₂CH₂O) m′ (CH₂)_{n″}CH₂, where n″ is selected from 0 to 10 and m′ is selected from 0 to 4 and wherein the structure in square backet represents a repeat unit of the serotype 22A saccharide and wherein n represents the number of repeating units.

87. A serotype 22A glycoconjugate comprising a serotype 22A saccharide covalently conjugated to a carrier protein (CP) through a spacer and having the general formula (IV), wherein X is CH₂(CH₂)_{n′}, where n′ is 0 and wherein X′ is CH₂(CH₂)_{n″} where n″ is 0.

88. A serotype 22A glycoconjugate comprising a serotype 22A saccharide covalently conjugated to a carrier protein (CP) through a spacer and having the general formula (VII),

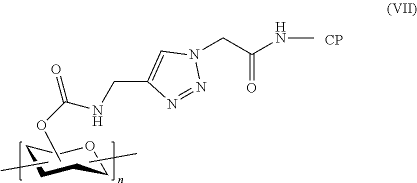

wherein the structure in square backet represents a repeat unit of the serotype 22A saccharide and wherein n represents the number of repeating units.

89. The glycoconjugate of any one of paragraphs 31 to 88 wherein said a serotype 22A saccharide is an isolated S. pneumoniae serotype 22A saccharide of any one of paragraphs 1-28.

90. The glycoconjugate of any one of paragraphs 29 to 88 wherein the weight average molecular weight (Mw) of said S. pneumoniae serotype 22A saccharide before conjugation is between 50 kDa and 1,000 kDa.

91. The glycoconjugate of any one of paragraphs 29 to 88 wherein the weight average molecular weight (Mw) of said S. pneumoniae serotype 22A saccharide before conjugation is between 100 kDa and 600 kDa.

92. The glycoconjugate of any one of paragraphs 29 to 88 wherein the weight average molecular weight (Mw) of said S. pneumoniae serotype 22A saccharide before conjugation is between 100 kDa and 400 kDa.

93. The glycoconjugate of any one of paragraphs 29 to 88 wherein the weight average molecular weight (Mw) of said S. pneumoniae serotype 22A saccharide before conjugation is between 150 kDa and 300 kDa.

94. The glycoconjugate of any one of paragraphs 29 to 93 wherein said serotype 22A glycoconjugate has a weight average molecular weight (Mw) of between 250 kDa and 20,000 kDa.

95. The glycoconjugate of any one of paragraphs 29 to 93 wherein said serotype 22A glycoconjugate has a weight average molecular weight (Mw) of between 500 kDa and 15,000 kDa.

96. The glycoconjugate of any one of paragraphs 29 to 93 wherein said serotype 22A glycoconjugate has a weight average molecular weight (Mw) of between 500 kDa and 10,000 kDa.

97. The glycoconjugate of any one of paragraphs 29 to 93 wherein said serotype 22A glycoconjugate has a weight average molecular weight (Mw) of between 750 kDa and 7,500 kDa.

98. The glycoconjugate of any one of paragraphs 29 to 93 wherein said serotype 22A glycoconjugate has a weight average molecular weight (Mw) of between 1,000 kDa and 5,000 kDa.

99. The glycoconjugate of any one of paragraphs 29 to 98 wherein the degree of conjugation of said serotype 22A glycoconjugate is between 2 and 15.

100. The glycoconjugate of any one of paragraphs 29 to 98 wherein the degree of conjugation of said serotype 22A glycoconjugate is between 2 and 10.

101. The glycoconjugate of any one of paragraphs 29 to 98 wherein the degree of conjugation of said serotype 22A glycoconjugate is between 3 and 5.

102. The glycoconjugate of any one of paragraphs 29 to 98 wherein the degree of conjugation of said serotype 22A glycoconjugate is between 2 and 6.

103. The glycoconjugate of any one of paragraphs 29 to 98 wherein the degree of conjugation of said serotype 22A glycoconjugate is between 4 and 10.

104. The glycoconjugate of any one of paragraphs 29 to 103 wherein the ratio of serotype 22A saccharide to carrier protein in the glycoconjugate (w/w) is between 0.5 and 3.0.

105. The glycoconjugate of any one of paragraphs 29 to 103 wherein the ratio of serotype 22A saccharide to carrier protein in the glycoconjugate (w/w) is between 0.5 and 2.0.

106. The glycoconjugate of any one of paragraphs 29 to 103 wherein the ratio of serotype 22A saccharide to carrier protein in the glycoconjugate (w/w) is between 0.5 and 1.5.

107. The glycoconjugate of any one of paragraphs 29 to 103 wherein the ratio of serotype 22A saccharide to carrier protein in the glycoconjugate (w/w) is between 0.8 and 1.2.

108. The glycoconjugate of any one of paragraphs 29 to 103 wherein the ratio of serotype 22A saccharide to carrier protein in the glycoconjugate (w/w) is between 0.5 and 1.0.

109. The glycoconjugate of any one of paragraphs 29 to 103 wherein the ratio of serotype 22A saccharide to carrier protein in the glycoconjugate (w/w) is between 1.0 and 1.5.

110. The glycoconjugate of any one of paragraphs 29 to 103 wherein the ratio of serotype 22A saccharide to carrier protein in the glycoconjugate (w/w) is between 1.0 and 2.0.

111. The glycoconjugate of any one of paragraphs 29 to 103 wherein the ratio of serotype 22A saccharide to carrier protein in the glycoconjugate (w/w) is between 0.8 and 1.2.

112. The glycoconjugate of any one of paragraphs 29 to 103 wherein the ratio of serotype 22A saccharide to carrier protein in the glycoconjugate (w/w) is between 0.7 and 1.1.

113. The glycoconjugate of any one of paragraphs 29 to 112 wherein said serotype 22A glycoconjugate comprises less than about 50% of free serotype 22A saccharide compared to the total amount of serotype 22A saccharide.

114. The glycoconjugate of any one of paragraphs 29 to 112 wherein said serotype 22A glycoconjugate comprises less than about 25% of free serotype 22A saccharide compared to the total amount of serotype 22A saccharide.

115. The glycoconjugate of any one of paragraphs 29 to 112 wherein said serotype 22A glycoconjugate comprises less than about 20% of free serotype 22A saccharide compared to the total amount of serotype 22A saccharide.

116. The glycoconjugate of any one of paragraphs 29 to 112 wherein said serotype 22A glycoconjugate comprises less than about 15% of free serotype 22A saccharide compared to the total amount of serotype 22A saccharide.

117. The glycoconjugate of any one of paragraphs 29 to 112 wherein at least 30% of the serotype 22A glycoconjugate has a K_{d} below or equal to 0.3 in a CL-4B column.

118. The glycoconjugate of any one of paragraphs 29 to 112 wherein at least 40% of the glycoconjugate has a K_{d} below or equal to 0.3 in a CL-4B column.

119. The glycoconjugate of any one of paragraphs 29 to 116 wherein at least 60% of the serotype 22A glycoconjugate has a K_{d} below or equal to 0.3 in a CL-4B column.

120. The glycoconjugate of any one of paragraphs 29 to 116 wherein between 50% and 80% of the serotype 22A glycoconjugate has a K_{d} below or equal to 0.3 in a CL-4B column.

121. The glycoconjugate of any one of paragraphs 29 to 116 wherein between 65% and 80% of the serotype 22A glycoconjugate has a K_{d} below or equal to 0.3 in a CL-4B column.

122. The glycoconjugate of any one of paragraphs 29 to 121 wherein the carrier protein of the serotype 22A saccharide glycoconjugate is selected in the group consisting of: TT, DT, DT mutants and a C5a peptidase from Streptococcus (SCP).

123. The glycoconjugate of any one of paragraphs 29 to 121 wherein the carrier protein of the serotype 22A saccharide glycoconjugate is rhizavidin [aa 45-179J-GGGGSSS-SP1500-AAA-SP0785] (CP1).

123a. The glycoconjugate of any one of paragraphs 29 to 121 wherein the carrier protein of the serotype 22A saccharide glycoconjugate is Rhavi-linker-PdT (G294P)-linker-SP0435 [aa 62-185] fusion protein (SPP2).

124. The glycoconjugate of any one of paragraphs 29 to 121 wherein the carrier protein of the serotype 22A saccharide glycoconjugate is DT.

125. The glycoconjugate of any one of paragraphs 29 to 121 wherein the carrier protein of the serotype 22A saccharide glycoconjugate is TT.

126. The glycoconjugate of any one of paragraphs 29 to 121 wherein the carrier protein of the serotype 22A saccharide glycoconjugate is PD.

127. The glycoconjugate of any one of paragraphs 29 to 121 wherein the carrier protein of the serotype 22A saccharide glycoconjugate is CRM₁₉₇ or a C5a peptidase from Streptococcus (SCP).

128. The glycoconjugate of any one of paragraphs 29 to 121 wherein the carrier protein of the serotype 22A saccharide glycoconjugate is CRM₁₉₇.

129. The glycoconjugate of any one of paragraphs 29 to 121 wherein the carrier protein of the serotype 22A saccharide glycoconjugate is SCP.

130. The glycoconjugate of any one of paragraphs 29 to 121 wherein the carrier protein of the serotype 22A saccharide glycoconjugate is an enzymatically inactive SCP.

131. The glycoconjugate of any one of paragraphs 29 to 121 wherein the carrier protein of the serotype 22A saccharide glycoconjugate is an enzymatically inactive SCP from GBS (SCPB).

132. The glycoconjugate of any one of paragraphs 29 to 121 wherein the carrier protein of the serotype 22A saccharide glycoconjugate is an enzymatically inactive SCP from GAS (SCPA).

133. The glycoconjugate of any one of paragraphs 29 to 121 wherein the carrier protein of the serotype 22A saccharide glycoconjugate is a fragment of an SCPB.

134. The glycoconjugate of any one of paragraphs 29 to 121 wherein the carrier protein of the serotype 22A saccharide glycoconjugate is an enzymatically inactive fragment of SCP which consists of SEQ ID NO: 1.

135. The glycoconjugate of any one of paragraphs 29 to 121 wherein the carrier protein of the serotype 22A saccharide glycoconjugate is an enzymatically inactive fragment of SCP which consists of SEQ ID NO: 2.

136. An immunogenic composition comprising a S. pneumoniae serotype 22A saccharide according to any one of paragraphs 1-28.

137. An immunogenic composition comprising a S. pneumoniae serotype 22A saccharide glycoconjugate according to any one of paragraphs 29-135.

138. The immunogenic composition of paragraph 137 comprising from 1 to 45 different glycoconjugates.

139. The immunogenic composition of paragraph 137 comprising from 1 to 45 glycoconjugates from different serotypes of S. pneumoniae.

140. The immunogenic composition of paragraph 137 comprising glycoconjugates from 7, 8, 9, 10, 11, 12, 13, 14, 15, 16, 17, 18, 19, 20, 21, 22, 23, 24 or 25 different serotypes of S. pneumoniae.

141. The immunogenic composition of paragraph 137 comprising glycoconjugates from 16 or 20 different serotypes of S. pneumoniae.

142. The immunogenic composition of paragraph 137 which is a 21, 22, 23, 24 or 25-valent pneumococcal conjugate composition.

143. The immunogenic composition of paragraph 137 which is a 21-valent pneumococcal conjugate composition.

144. The immunogenic composition of paragraph 137 which is a 22-valent pneumococcal conjugate composition.

145. The immunogenic composition of paragraph 137 which is a 23-valent pneumococcal conjugate composition.

146. The immunogenic composition of paragraph 137 which is a 24-valent pneumococcal conjugate composition.

147. The immunogenic composition of paragraph 137 which is a 25-valent pneumococcal conjugate composition.

148. The immunogenic composition of paragraph 137 comprising glycoconjugates from 26 to 45 glycoconjugates from different serotypes of S. pneumoniae.

149. The immunogenic composition of paragraph 137 comprising glycoconjugates from 26, 27, 28, 29, 30, 31, 32, 33, 34, 35, 36, 37, 38, 39, 40, 41, 42, 43, 44 or 45 different serotypes of S. pneumoniae.

150. The immunogenic composition of paragraph 137 comprising glycoconjugates from 35 or 45 different serotypes of S. pneumoniae.

151. The immunogenic composition of paragraph 137 which is a 35, 36, 37, 38, 39, 40, 41, 42, 43, 44 or 45-valent pneumococcal conjugate composition.

152. The immunogenic composition of paragraph 137 which is a 40, 41, 42, 43, 44 or 45-valent pneumococcal conjugate composition.

153. The immunogenic composition of paragraph 137 which is a 40-valent pneumococcal conjugate composition.

154. The immunogenic composition of paragraph 137 which is a 41-valent pneumococcal conjugate composition.

155. The immunogenic composition of paragraph 137 which is a 42-valent pneumococcal conjugate composition.

156. The immunogenic composition of paragraph 137 which is a 43-valent pneumococcal conjugate composition.

157. The immunogenic composition of paragraph 137 which is a 44-valent pneumococcal conjugate composition.

158. The immunogenic composition of paragraph 137 which is a 45-valent pneumococcal conjugate composition.

159. The immunogenic composition of any one of paragraphs 137-158 further comprising glycoconjugates from S. pneumoniae serotypes 4, 6B, 9V, 14, 18C, 19F and 23F.

160. The immunogenic composition of paragraph 159 further comprising glycoconjugates from S. pneumoniae serotypes 1, 5 and 7F.

161. The immunogenic composition of paragraph 160 further comprising a glycoconjugate from S. pneumoniae serotype 3.

162. The immunogenic composition of paragraph 161 further comprising glycoconjugates from S. pneumoniae serotypes 6A and 19A.

163. The immunogenic composition of paragraph 162 further comprising glycoconjugates from S. pneumoniae serotype 22F and 33F.

164. The immunogenic composition of paragraph 163 further comprising glycoconjugates from S. pneumoniae serotypes 8, 10A, 11A, 12F and 15B.

165. The immunogenic composition of paragraph 164 further comprising a glycoconjugate from S. pneumoniae serotype 2.

166. The immunogenic composition of paragraph 165 further comprising a glycoconjugate from S. pneumoniae serotype 9N.

167. The immunogenic composition of paragraph 166 further comprising a glycoconjugate from S. pneumoniae serotype 17F.

168. The immunogenic composition of paragraph 167 further comprising a glycoconjugate from S. pneumoniae serotype 20.

169. The immunogenic composition of paragraph 168 further comprising a glycoconjugate from S. pneumoniae serotype 2.

170. The immunogenic composition of paragraph 169 further comprising a glycoconjugate from S. pneumoniae serotype 15C.

171. The immunogenic composition of any one of paragraphs 137-140 further comprising glycoconjugates from S. pneumoniae serotypes 4, 6B, 9V, 14, 18C, 19F and 23F and wherein said immunogenic composition is an 8-valent pneumococcal conjugate composition.

172. The immunogenic composition of any one of paragraphs 137-140 further comprising glycoconjugates from S. pneumoniae serotypes 1, 4, 5, 6B, 7F, 9V, 14, 18C, 19F and 23F and wherein said immunogenic composition is an 11-valent pneumococcal conjugate composition.

173. The immunogenic composition of any one of paragraphs 137-140 further comprising glycoconjugates from S. pneumoniae serotypes 1, 3, 4, 5, 6A, 6B, 7F, 9V, 14, 18C, 19A, 19F and 23F and wherein said immunogenic composition is a 14-valent pneumococcal conjugate composition.

174. The immunogenic composition of any one of paragraphs 137-140 further comprising glycoconjugates from S. pneumoniae serotypes 1, 3, 4, 5, 6A, 6B, 7F, 9V, 14, 18C, 19A, 19F, 22F, 23F and 33F and wherein said immunogenic composition is a 16-valent pneumococcal conjugate composition.

175. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and wherein said immunogenic composition is a 21-valent pneumococcal conjugate composition.

176. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15C, 18C, 19A, 19F, 22F, 23F and 33F.

177. The immunogenic composition of paragraph 176 which is a 21-valent pneumococcal conjugate composition.

178. The immunogenic composition of any one of paragraphs 137-140 further comprising glycoconjugates from S. pneumoniae serotypes 1, 2, 3, 4, 5, 6A, 6B, 7F, 8, 9V, 10A, 11A, 12F, 14, 15B, 18C, 19A, 19F, 22F, 23F and 33F.

179. The immunogenic composition of paragraph 178 which is a 22-valent pneumococcal conjugate composition.

180. The immunogenic composition of any one of paragraphs 137-140 further comprising glycoconjugates from S. pneumoniae serotypes 1, 2, 3, 4, 5, 6A, 6B, 7F, 8, 9V, 10A, 11A, 12F, 14, 15C, 18C, 19A, 19F, 22F, 23F and 33F.

181. The immunogenic composition of paragraph 180 which is a 22-valent pneumococcal conjugate composition.

182. The immunogenic composition of any one of paragraphs 137-140 further comprising glycoconjugates from S. pneumoniae serotypes 1, 3, 4, 5, 6A, 6B, 7F, 8, 9V, 9N, 10A, 11A, 12F, 14, 15B, 18C, 19A, 19F, 22F, 23F and 33F.

183. The immunogenic composition of paragraph 182 which is a 22-valent pneumococcal conjugate composition.

184. The immunogenic composition of any one of paragraphs 137-140 further comprising glycoconjugates from S. pneumoniae serotypes 1, 2, 3, 4, 5, 6A, 6B, 7F, 8, 9V, 9N, 10A, 11A, 12F, 14, 15B, 18C, 19A, 19F, 22F, 23F and 33F.

185. The immunogenic composition of paragraph 184 which is a 23-valent pneumococcal conjugate composition.

186. The immunogenic composition of any one of paragraphs 137-140 further comprising glycoconjugates from S. pneumoniae serotypes 1, 2, 3, 4, 5, 6A, 6B, 7F, 8, 9V, 9N, 10A, 11A, 12F, 14, 15B, 17F, 18C, 19A, 19F, 22F, 23F and 33F.

187. The immunogenic composition of paragraph 186 which is a 24-valent pneumococcal conjugate composition.

188. The immunogenic composition of any one of paragraphs 137-140 further comprising glycoconjugates from S. pneumoniae serotypes 1, 2, 3, 4, 5, 6A, 6B, 7F, 8, 9V, 9N, 10A, 11A, 12F, 14, 15B, 17F, 18C, 19A, 19F, 20, 22F, 23F and 33F.

189. The immunogenic composition of paragraph 188 which is a 25-valent pneumococcal conjugate composition.

190. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F.

191. The immunogenic composition of paragraph 190 which is a 22-valent pneumococcal conjugate composition.

192. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F.

193. The immunogenic composition of paragraph 192 which is a 22-valent pneumococcal conjugate composition.

194. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F.

195. The immunogenic composition of paragraph 194 which is a 22-valent pneumococcal conjugate composition.

196. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F and 33F.

197. The immunogenic composition of paragraph 196 which is a 22-valent pneumococcal conjugate composition.

198. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B.

199. The immunogenic composition of paragraph 198 which is a 22-valent pneumococcal conjugate composition.

200. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F and 33F.

201. The immunogenic composition of paragraph 200 which is a 23-valent pneumococcal conjugate composition.

202. The immunogenic composition of any one of paragraphs 137-140 further comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B.

203. The immunogenic composition of paragraph 202 wherein the S. pneumoniae saccharides are conjugated to CRM₁₉₇.

204. The immunogenic composition of paragraph 202 wherein the S. pneumoniae saccharides from serotypes 1, 4, 5, 6A, 6B, 7F, 8, 9V, 10A, 11A, 12F, 14, 15A, 15B, 18C, 19A, 19F, 22F, 23A, 23B, 23F, 24F, 33F, 35B and 38 are conjugated to CRM₁₉₇ and the S. pneumoniae saccharide serotype 3 is conjugated to SCP.

205. The immunogenic composition of paragraph 204 which is a 26-valent pneumococcal conjugate composition.

206. The immunogenic composition of any one of paragraphs 137-140 further comprising at least one glycoconjugate selected from the group consisting of glycoconjugates from S. pneumoniae serotypes 2, 3, 7C, 9N, 10B, 15A, 16F, 17F, 20, 21, 23A, 23B, 24B, 24F, 27, 29, 31, 33B, 34, 35B, 35F and 38.

207. The immunogenic composition of any one of paragraphs 137-140 further comprising twenty-one glycoconjugates selected from the group consisting of glycoconjugates from S. pneumoniae serotypes 2, 3, 7C, 9N, 10B, 15A, 16F, 17F, 20, 21, 23A, 23B, 24B, 24F, 27, 29, 31, 33B, 34, 35B, 35F and 38.

208. The immunogenic composition of paragraph 207 which is a 22-valent pneumococcal conjugate composition.

209. The immunogenic composition of any one of paragraphs 137-140 further comprising glycoconjugates from serotypes 2, 3, 7C, 9N, 10B, 15A, 16F, 17F, 20, 21, 23A, 23B, 24B, 24F, 27, 29, 31, 33B, 34, 35B, 35F and 38.

210. The immunogenic composition of paragraph 209 which is a 23-valent pneumococcal conjugate composition.

211. The immunogenic composition of any one of paragraphs 137-140 further comprising at least one glycoconjugate selected from the group consisting of glycoconjugates from S. pneumoniae serotypes 2, 3, 7C, 9N, 10B, 15A, 16F, 17F, 19A, 19F, 20, 21, 23A, 23B, 24B, 24F, 27, 29, 31, 33B, 34, 35B, 35F and 38.

212. The immunogenic composition of any one of paragraphs 137-140 further comprising twenty-two glycoconjugates selected from the group consisting of glycoconjugates from S. pneumoniae serotypes 2, 3, 7C, 9N, 10B, 15A, 16F, 17F, 19A, 19F, 20, 21, 23A, 23B, 24B, 24F, 27, 29, 31, 33B, 34, 35B, 35F and 38.

213. The immunogenic composition of paragraph 212 which is a 23-valent pneumococcal conjugate composition.

214. The immunogenic composition of any one of paragraphs 137-140 further comprising twenty-three glycoconjugates selected from the group consisting of glycoconjugates from S. pneumoniae serotypes 2, 3, 7C, 9N, 10B, 15A, 16F, 17F, 19A, 19F, 20, 21, 23A, 23B, 24B, 24F, 27, 29, 31, 33B, 34, 35B, 35F and 38.

215. The immunogenic composition of paragraph 214 which is a 24-valent pneumococcal conjugate composition.

216. The immunogenic composition of any one of paragraphs 137-140 further comprising glycoconjugates from serotypes 2, 3, 7C, 9N, 10B, 15A, 16F, 17F, 20, 21, 23A, 23B, 24B, 24F, 27, 29, 31, 33B, 34, 35B, 35F and 38.

217. The immunogenic composition of paragraph 216 which is a 23-valent pneumococcal conjugate composition.

218. The immunogenic composition of any one of paragraphs 137-140 further comprising glycoconjugates from serotypes 2, 3, 7C, 9N, 10B, 15A, 16F, 17F, 19A, 19F, 20, 21, 23A, 23B, 24B, 24F, 27, 29, 31, 33B, 34, 35B, 35F and 38.

219. The immunogenic composition of paragraph 210 which is a 25-valent pneumococcal conjugate composition.

220. The immunogenic composition of any one of paragraphs 137-219 further comprising at least one, two or three adjuvants.

221. The immunogenic composition of any one of paragraphs 128-211 further comprising one adjuvant.

222. The immunogenic composition of any one of paragraphs 137-219 further comprising two adjuvants.

223. The immunogenic composition of any one of paragraphs 137-219 Further comprising aluminum salts (alum) as adjuvant.

224. The immunogenic composition of any one of paragraphs 137-219 further comprising aluminum phosphate as adjuvant.

225. The immunogenic composition of any one of paragraphs 137-219 further comprising a saponin based adjuvant.

226. The immunogenic composition of any one of paragraphs 137-219 further comprising a QS21 based adjuvant.

227. The immunogenic composition of any one of paragraphs 137-219 further comprising a CpG Oligonucleotide as adjuvant.

228. The immunogenic composition of any one of paragraphs 137-227 for use as a vaccine.

229. The immunogenic composition of any one of paragraphs 137-227 for use in a method of preventing, treating or ameliorating an infection, disease or condition associated with S. pneumoniae serotype 22A in a subject.

230. The immunogenic composition of any one of paragraphs 137-227 for use in a method of inducing an immune response to S. pneumoniae serotype 22A in a subject said method comprising administering to the subject an immunologically effective amount of said immunogenic composition.

231. The immunogenic composition of any one of paragraphs 137-227 for use in a method of preventing an infection by S. pneumoniae 22A in a subject said method comprising administering to the subject an immunologically effective amount of said immunogenic composition.

232. A method of detecting the presence of O-acetyl groups in an isolated S. pneumoniae serotype 22A polysaccharide, said method comprising the step of: a) isolating an S. pneumoniae serotype 22A polysaccharide and b) detecting the presence of O-acetyl groups in said polysaccharide.

233. The method of paragraph 232 wherein the presence of O-acetyl groups is detected by NMR, Mass Spectrometry (MS) or High-performance liquid chromatography (HPLC).

234. The method of paragraph 232 wherein the presence of O-acetyl groups is detected by NMR. 235. The method of paragraph 232 wherein the presence of O-acetyl groups is detected by Mass Spectrometry (MS).

236. The method of paragraph 232 wherein the presence of O-acetyl groups is detected by High-performance liquid chromatography (HPLC).

237. A method of detecting the presence of O-acetyl groups in an oxidized serotype 22A polysaccharide, said method comprising the step of: a) reacting an isolated S. pneumoniae serotype 22A polysaccharide with an oxidizing agent and b) detecting the presence of O-acetyl groups in said oxidized polysaccharide.

238. The method of paragraph 237 wherein the presence of O-acetyl groups is detected by NMR, Mass Spectrometry (MS) or High-performance liquid chromatography (HPLC).

239. The method of paragraph 237 wherein the presence of O-acetyl groups is detected by NMR. 240. The method of paragraph 237 wherein the presence of O-acetyl groups is detected by Mass Spectrometry (MS).

241. The method of paragraph 237 wherein the presence of O-acetyl groups is detected by High-performance liquid chromatography (HPLC).

242. The method of any one of paragraphs 237-241 wherein said oxidizing agent is any oxidizing agent which oxidizes a terminal hydroxyl group to an aldehyde.

243. The method of any one of paragraphs 237-241 wherein said oxidizing agent is periodate.

244. The method of any one of paragraphs 237-241 wherein said oxidizing agent is orthoperiodate.

245. The method of any one of paragraphs 237-241 wherein said oxidizing agent is sodium periodate.

246. The method of any one of paragraphs 237-241 wherein said oxidizing agent is metaperiodate.

247. The method of any one of paragraphs 237-241 wherein said oxidizing agent is sodium metaperiodate.

248. The method of any one of paragraphs 237-241 wherein said oxidizing agent is a stable nitroxyl radical compound and an oxidant.

249. The method of paragraph 248 wherein said stable nitroxyl radical compound is a molecule bearing a TEMPO or a PROXYL (2,2,5,5-tetramethyl-1-pyrrolidinyloxy) moiety.

250. The method of paragraph 248 wherein said stable nitroxyl radical compound is 2,2,6,6-Tetramethyl-1-piperidinyloxy free radical (TEMPO) and said oxidant is N-Chlorosuccinimide (NCS).

251. A method of detecting the presence of O-acetyl groups in S. pneumoniae serotype 22A glycoconjugate, said method comprising the step of: a) preparing a S. pneumoniae serotype 22A glycoconjugate and b) detecting the presence of O-acetyl groups in said glycoconjugate.

252. The method of paragraph 251 wherein the presence of O-acetyl groups is detected by NMR, Mass Spectrometry (MS) or High-performance liquid chromatography (HPLC).

253. The method of paragraph 251 wherein the presence of O-acetyl groups is detected by NMR. 254. The method of paragraph 251 wherein the presence of O-acetyl groups is detected by Mass Spectrometry (MS).

255. The method of paragraph 251 wherein the presence of O-acetyl groups is detected by High-performance liquid chromatography (HPLC).

256. A method of measuring the degree of O-acetylation of an isolated S. pneumoniae serotype 22A polysaccharide, said method comprising the step of: a) isolating an S. pneumoniae serotype 22A polysaccharide and b) measuring the amount of O-acetyl groups in said polysaccharide.

257. The method of paragraph 256 wherein the amount of O-acetyl groups is measured by NMR, Mass Spectrometry (MS) or High-performance liquid chromatography (HPLC).

258. The method of paragraph 256 wherein the amount of O-acetyl groups is measured by NMR.

259. The method of paragraph 256 wherein the amount of O-acetyl groups is measured by Mass Spectrometry (MS).

260. The method of paragraph 256 wherein the amount of O-acetyl groups is measured by High-performance liquid chromatography (HPLC).

261. A method of measuring the degree of O-acetylation of an isolated S. pneumoniae serotype 22A polysaccharide, said method comprising the step of: a) isolating an S. pneumoniae serotype 22A polysaccharide b) treating said polysaccharide with a base and c) measuring the amount of O-acetyl groups in said polysaccharide.

262. The method of paragraph 261 wherein the amount of O-acetyl groups is measured by NMR, Mass Spectrometry (MS) or High-performance liquid chromatography (HPLC).

263. The method of paragraph 261 wherein the amount of O-acetyl groups is measured by NMR. 264. The method of paragraph 261 wherein the amount of O-acetyl groups is measured by Mass Spectrometry (MS).

265. The method of paragraph 261 wherein the amount of O-acetyl groups is measured by High-performance liquid chromatography (HPLC).

266. A method of measuring the degree of O-acetylation in an oxidized serotype 22A polysaccharide, said method comprising the step of: a) reacting an isolated S. pneumoniae serotype 22A polysaccharide with an oxidizing agent and b) measuring the amount of O-acetyl groups in said oxidized polysaccharide.

267. The method of paragraph 266 wherein the amount of O-acetyl groups is measured by NMR, Mass Spectrometry (MS) or High-performance liquid chromatography (HPLC).

268. The method of paragraph 266 wherein the amount of O-acetyl groups is measured by NMR.

269. The method of paragraph 266 wherein the amount of O-acetyl groups is measured by Mass Spectrometry (MS).

270. The method of paragraph 266 wherein the amount of O-acetyl groups is measured by High-performance liquid chromatography (HPLC).

271. The method of any one of paragraphs 266-270 wherein said oxidizing agent is any oxidizing agent which oxidizes a terminal hydroxyl group to an aldehyde.

272. The method of any one of paragraphs 266-270 wherein said oxidizing agent is periodate.

273. The method of any one of paragraphs 266-270 wherein said oxidizing agent is orthoperiodate.

274. The method of any one of paragraphs 266-270 wherein said oxidizing agent is sodium periodate.

275. The method of any one of paragraphs 266-270 wherein said oxidizing agent is metaperiodate.

276. The method of any one of paragraphs 266-270 wherein said oxidizing agent is sodium metaperiodate.

277. The method of any one of paragraphs 266-2702 wherein said oxidizing agent is a stable nitroxyl radical compound and an oxidant.

278. The method of paragraph 277 wherein said stable nitroxyl radical compound is a molecule bearing a TEMPO or a PROXYL (2,2,5,5-tetramethyl-1-pyrrolidinyloxy) moiety.

279. The method of paragraph 277 wherein said stable nitroxyl radical compound is 2,2,6,6-Tetramethyl-1-piperidinyloxy free radical (TEMPO) and said oxidant is N-Chlorosuccinimide (NCS).

280. A method of measuring the degree of O-acetylation in S. pneumoniae serotype 22A glycoconjugate, said method comprising the step of: a) preparing a S. pneumoniae serotype 22A glycoconjugate and b) measuring the amount of O-acetyl groups in said glycoconjugate.

281. The method of paragraph 280 wherein the amount of O-acetyl groups is measured by NMR, Mass Spectrometry (MS) or High-performance liquid chromatography (HPLC).

282. The method of paragraph 280 wherein the amount of O-acetyl groups is measured by NMR. 283. The method of paragraph 280 wherein the amount of O-acetyl groups is measured by Mass Spectrometry (MS).

284. The method of paragraph 280 wherein the amount of O-acetyl groups is measured by High-performance liquid chromatography (HPLC).

285. A method of detecting the presence of L-Arabinofuranose-5-Aldehyde residues in an oxidized serotype 22A polysaccharide, said method comprising the step of: a) reacting an isolated S. pneumoniae serotype 22A polysaccharide with an oxidizing agent and b) detecting the presence of L-Arabinofuranose-5-Aldehyde residues in said oxidized polysaccharide.

286. The method of paragraph 285 wherein the presence of L-Arabinofuranose-5-Aldehyde residues is detected by NMR.

287. The method of paragraph 285 wherein the presence of L-Arabinofuranose-5-Aldehyde residues is detected by 2D NMR.

288. The method of paragraph 285 wherein the presence of L-Arabinofuranose-5-Aldehyde residues is detected by Mass Spectrometry (MS).

289. The method of paragraph 285 wherein the presence of L-Arabinofuranose-5-Aldehyde residues is detected by Hydrophilic Interaction Liquid Chromatography-Mass Spectrometry (HILIC-LC/MS).

290. The method of any one of paragraphs 285-289 wherein said oxidizing agent is any oxidizing agent which oxidizes a terminal hydroxyl group to an aldehyde.

291. The method of any one of paragraphs 285-289 wherein said oxidizing agent is periodate.

292. The method of any one of paragraphs 285-289 wherein said oxidizing agent is orthoperiodate.

293. The method of any one of paragraphs 285-289 wherein said oxidizing agent is sodium periodate.

294. The method of any one of paragraphs 285-289 wherein said oxidizing agent is metaperiodate.

295. The method of any one of paragraphs 285-289 wherein said oxidizing agent is sodium metaperiodate.

296. The method of any one of paragraphs 285-289 wherein said oxidizing agent is a stable nitroxyl radical compound and an oxidant.

297. The method of paragraph 296 wherein said stable nitroxyl radical compound is a molecule bearing a TEMPO or a PROXYL (2,2,5,5-tetramethyl-1-pyrrolidinyloxy) moiety.

298. The method of paragraph 296 wherein said stable nitroxyl radical compound is 2,2,6,6-Tetramethyl-1-piperidinyloxy free radical (TEMPO) and said oxidant is N-Chlorosuccinimide (NCS).

299. A method of detecting the presence of α-L-Arabinofuranose (α-L-Araf) residues in a reduced serotype 22A polysaccharide, said method comprising the step of: a) reacting an isolated S. pneumoniae serotype 22A polysaccharide with a reducing agent and b) detecting the presence of α-L-Arabinofuranose (α-L-Araf) residues in said reduced polysaccharide.

300. The method of paragraph 299 wherein the presence of α-L-Arabinofuranose (α-L-Araf) residues is detected by NMR.

301. The method of paragraph 299 wherein the presence of α-L-Arabinofuranose (α-L-Araf) residues is detected by 2D NMR.

302. The method of paragraph 299 wherein the presence of α-L-Arabinofuranose (α-L-Araf) residues is detected by Heteronuclear Single Quantum Coherence Spectroscopy (HSQC), Heteronuclear multiple-bond correlation spectroscopy (HMBC), Correlation spectroscopy (COSY) or Heteronuclear Single Quantum Coherence Spectroscopy-Total Correlation Spectroscopy (HSQC-TOCSY).

303. The method of paragraph 299 wherein the presence of α-L-Arabinofuranose (α-L-Araf) residues is detected by 2D ¹H-¹³C HSQC NMR.

304. The method of paragraph 299 wherein the presence of α-L-Arabinofuranose (α-L-Araf) residues is detected by Mass Spectrometry (MS).

305. The method of any one of paragraphs 299-304 wherein the reducing agent is sodium borohydride (NaBH₄).

306. The method of any one of paragraphs 299-305 wherein said isolated S. pneumoniae serotype 22A polysaccharide has been previously treated with an oxidizing agent.

307. The method of paragraph 306 wherein the oxidizing agent is any oxidizing agent which oxidizes a terminal hydroxyl group to an aldehyde.

308. The method of paragraph 306 wherein the oxidizing agent is periodate.

309. The method of paragraph 306 wherein the oxidizing agent is orthoperiodate.

310. The method of paragraph 306 wherein the oxidizing agent is sodium periodate.

311. The method of paragraph 306 wherein the oxidizing agent is metaperiodate.

312. The method of paragraph 306 wherein the oxidizing agent is sodium metaperiodate.

313. The method of any one of paragraphs 299-305 wherein said isolated S. pneumoniae serotype 22A polysaccharide has been previously treated with a stable nitroxyl radical compound and an oxidant.

314. The method of paragraph 313 wherein said stable nitroxyl radical compound is a molecule bearing a TEMPO or a PROXYL (2,2,5,5-tetramethyl-1-pyrrolidinyloxy) moiety.

315. The method of paragraph 313 wherein said stable nitroxyl radical compound is 2,2,6,6-Tetramethyl-1-piperidinyloxy free radical (TEMPO) and said oxidant is N-Chlorosuccinimide (NCS).

316. A method of detecting the presence of α-L-Arabinofuranose (α-L-Araf) residues in S. pneumoniae serotype 22A glycoconjugate, said method comprising the step of: a) preparing a S. pneumoniae serotype 22A glycoconjugate and b) detecting the presence of α-L-Arabinofuranose (α-L-Araf) residues in said glycoconjugate.

317. The method of paragraph 316 wherein the presence of α-L-Arabinofuranose (α-L-Araf) residues is detected by NMR.

318. The method of paragraph 316 wherein the presence of α-L-Arabinofuranose (α-L-Araf) residues is detected by 2D NMR.

319. The method of paragraph 316 wherein the presence of α-L-Arabinofuranose (α-L-Araf) residues is detected by Heteronuclear Single Quantum Coherence Spectroscopy (HSQC), Heteronuclear multiple-bond correlation spectroscopy (HMBC), Correlation spectroscopy (COSY) or Heteronuclear Single Quantum Coherence Spectroscopy-Total Correlation Spectroscopy (HSQC-TOCSY).

320. The method of paragraph 316 wherein the presence of α-L-Arabinofuranose (α-L-Araf) residues is detected by 2D ¹H-¹³C HSQC NMR.

321. The method of paragraph 316 wherein the presence of α-L-Arabinofuranose (α-L-Araf) residues is detected by Mass Spectrometry (MS).

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLES

### Example 1: Serotype 22A Capsular Saccharides, Samples Preparation

Streptococcus pneumoniae Serotype 22A Polysaccharide:

25 mg of lyophilized native Streptococcus pneumoniae serotype 22A was weighed and dissolved in 1000 μL of D20. The sample was bath sonicated for 20 minutes at 50° C. The polysaccharide solution was sized using sonication for 10 minutes with 20% amplitude power for 15 second ON and 15 second OFF cycles. Final concentration of the native and sized samples was 25 mg/mL. The samples were transferred into the NMR tube for NMR data collection and analysis.

### Example 2: Structural Elucidation of Streptococcus pneumoniae Serotype 22A Polysaccharide by NMR Experiments

Both one-dimensional (1D) and two-dimensional (2D) NMR experiments were conducted. 1D ¹H spectrum was collected to identify the chemical shift fingerprint for each proton in the molecule. 2D homo- and hetero-nuclear spectra are collected to identify the chemical bond pattern between protons and carbons in the molecule, to map the backbone of the representative sugars in the serotype 22A polymer repeat unit, the inter-sugar glycosidic linkages, and to identify the location of the different functional groups on the sugar backbones.

All 1D and 2D data was collected at 75° C. using Bruker 600 MHz spectrometer equipped with BBO cryoprobe. The NMR data was processed using NvX and analyzed using MestraNova and NMRViewJ (NvJ) software (Methods Mol. Biol. 278, 313, 2004). The proton signals were referenced using the TMS signal set to 0 ppm, whereas the carbon signals were indirectly referenced against the TMS signal (0 ppm).

1D proton data was collected with 32 scans, with recycle delay of 5s. The following 2D NMR experiments were recorded: ¹H-¹H homonuclear COSY (Correlation Spectroscopy, 2048×512: total number of point in the ¹H and ¹³C dimensions, respectively, with 2 scans, recycle delay 1s), ¹H-¹³C HSQC (Heteronuclear Single Quantum Coherence, 2048×256: total number of point in the ¹H and ¹³C dimensions, respectively, with 4 scans, recycle delay 1s), ¹H-¹³C HSQC-TOCSY (Heteronuclear Single Quantum Coherence-Total Correlation Spectroscopy, 2048×256: total number of point in the 1H and 13C dimensions, respectively, with 48 scans, recycle delay 1 s mixing time of 120 ms), ¹H-¹³C HMBC (Heteronuclear Multiple Bond Correlation, 2048×256: total number of point in the ¹H and ¹³C dimensions, respectively, long range J~5-10 Hz, with 48 scans, recycle delay 1 s) and ¹H-¹³C HSQC-COSY (Heteronuclear Single Quantum Coherence-Correlation Spectroscopy, 2048×256: total number of point in the ¹H and ¹³C dimensions, respectively, with 48 scans, recycle delay 1s), ¹H-¹³C CLIP-HSQC (1H decoupled Heteronuclear Single Quantum Coherence, 2048×256: total number of point in the ¹H and ¹³C dimensions, respectively, with 8 scans, recycle delay 1 s). 1D ³¹P data was collected with 256 scans, with recycle delay of 5s. ¹H-³¹P HMBC (Heteronuclear Multiple Bond Correlation, 2048×32: total number of points in the ¹H and ³¹P dimensions, respectively, long range J~5-10 Hz).

Structural elucidation of the polysaccharides entails assigning all the resonances of the polymer repeat unit (RU). We achieve this by analyzing both one-dimensional (1D) and two-dimensional (2D) NMR experiments. 1D ¹H spectrum was collected to identify the chemical shift fingerprint for each proton in the molecule. 2D homo- and hetero-nuclear spectra are collected to identify the chemical bond pattern between protons and carbons in the molecule, to map the backbone of the representative sugars in the serotype 22A polymer repeat unit, the inter-sugar glycosidic linkages, and to identify the location of the different functional groups on the sugar backbones.

The chemical structure of Streptococcus pneumoniae serotype 22A is shown in FIG. 1. The serotype 22A polysaccharide is a hexa-saccharide: β-D-glucuronic acid (A), O-acetylated β-L-rhamnose (B), α-D-Glucose (C), α-D-galactofuranose (D), α-D-galactopyranose (F) and α-L-rhamnose (E). The main chain consists of five sugars and one branched sugar linked to 3 position of O-acetylated β-L-rhamnose (B).

### 1D ¹H NMR Analysis of the Serotype 22A Polysaccharide

The 1D ¹H NMR spectrum of native polysaccharide shown in FIG. 2 can be correlated to the polysaccharide backbone containing the 6 sugar residues. The anomeric protons of the β-D-GlcAp (Residue A), β-L-Rhap2OAc (Residue B), α-D-Glcp (Residue C), α-D-Galf (Residue D), α-L-Rhap (Residue E) and α-D-Galp (Residue F) are at δ 4.67 ppm, δ 5.08 ppm, δ 4.99 ppm, 0 δ.05 ppm, δ 4.87 ppm and δ 5.11 ppm, respectively. The inset table in FIG. 2 shows the excellent agreement between the normalized area under the anomeric proton peaks and the expected theoretical area. The area under these peaks were deconvoluted using the MestraNova software.

The anomers α or β of each sugar units were determined using the three-bond ¹H-¹H coupling constant (³J) measured from anomeric proton signals and the ¹H-¹³C coupling constant (¹J_{CH}) of the anomeric carbon and proton. FIG. 3 and Table 1 show the ¹J_{CH} and ³J_{HH} coupling constants for the six anomeric resonances for serotype 22A, which were used to determine the structure of serotype 22A.

TABLE 1
¹J_{CH} and ³J_{HH} coupling constants for the
six anomeric resonances for serotype 22A.

| | ¹J_{CH} (Hz) | ³J_{HH} (Hz) |
|---|---|---|
| D-β-GlcAp (A) | 165 | 7.7 |
| L-β-Rhap2OAc (B) | 163 | — |
| D-α-Glcp (C) | 172 | 3.3 |
| D-α-Galf (D) | 175 | 4.1 |
| L-α-Rhap (E) | 171 | — |
| D-α-Galp (F) | 173 | 3.7 |

### 2D NMR Analysis of the Serotype 22A Polysaccharide

The complete structural elucidation of sized serotype 22A polysaccharide was achieved by doing the complete assignment of all the resonances of each sugar unit that are present in the repeat unit. Each of the sugar units were assigned using 2D heteronuclear NMR experiments that use short range ¹H-¹H (HSQC-COSY), long range (multiple bonds) ¹H-¹H (HSQC-TOCSY) or ¹³C-¹³C (HMBC) correlation to map the resonances within the sugar ring.

The 2D HSQC experiment provides the correlation between the ¹H and ¹³C signals that are directly bonded and is very sensitive for analyzing the polysaccharides. 2D HSQC-COSY yields a ¹H-¹H short range and HSQC-TOCSY yields a ¹H-¹H long range (mixing time ~120 ms) proton correlation and is very useful in assigning the resonances within the sugar ring. Similarly, the inverse detected 2D ¹H-¹³C HMBC experiment gives rise to cross-peaks between proton and carbon atoms that are long range scalar coupled through multiple carbon bonds. The intensities of these cross peaks are reflected in the ²J_{C,H} or ³J_{C,H} values. Generally, three bond correlations from the anomeric carbon to the carbon at 3 and 5 positions are seen. Also quite prominent are the correlations to the neighboring carbon through the glycosidic bond, which are helpful in sequential assignment of the sugar units and their connectivity.

All the sugar backbone resonances were assigned by comparing the resonances from the short range (HSQC-COSY) and long range (HSQC-TOCSY), ¹H-¹³C HMBC NMR experiments.

The complete set of the resonances of all sugars of serotype 22A is tabulated in Table 2.

TABLE 2
Chemical shift assignment of serotype 22A polysaccharide.

22A

| | | ¹H (ppm) | ¹³C (ppm) |
|---|---|---|---|
| D-β-GlcpA (A) | A1 | 4.67 | 102.8 |
| | A2 | 3.36 | 74.0 |
| | A3 | 3.54 | 75.3 |
| | A4 | 3.67 | 80.3 |
| | A5 | 3.73 | 77.1 |
| | CO | | 175.5 |
| L-β-Rhap2OAc (B) | B1 | 5.09 | 99.4 |
| | B2 | 5.71 | 67.9 |
| | B3 | 4.09 | 73.6 |
| | B4 | 3.87 | 76.8 |
| | B5 | 3.58 | 72.0 |
| | B6 | 1.39 | 17.7 |
| | B-OAc | 2.19 | 20.8 |
| | CO | | 173.9 |
| D-α-Glcp (C) | C1 | 4.99 | 99.0 |
| | C2 | 3.56 | 71.8 |
| | C3 | 3.83 | 73.2 |
| | C4 | 3.68 | 77.4 |
| | C5 | 3.87 | 71.2 |
| | C6 | 3.82 | 61.0 |
| D-α-Galf (D) | D1 | 5.05 | 102.4 |
| | D2 | 4.28 | 75.2 |
| | D3 | 4.33 | 80.6 |
| | D4 | 4.09 | 80.5 |
| | D5 | 3.77 | 71.3 |
| | D6 | 3.66 | 63.4 |
| L-α-Rhap (E) | E1 | 4.87 | 99.5 |
| | E2 | 3.94 | 79.5 |
| | E3 | 3.80 | 69.9 |
| | E4 | 3.42 | 72.8 |
| | E5 | 3.99 | 69.6 |
| | E6 | 1.27 | 16.9 |
| D-α-Galp (F) | F1 | 5.11 | 93.7 |
| | F2 | 3.80 | 68.5 |
| | F3 | 3.91 | 70.0 |
| | F4 | 3.98 | 70.0 |
| | F5 | 4.27 | 71.4 |
| | F6 | 3.74 | 61.9 |

### Example 3: Structural Comparison of Native and Sized Serotype 22A Polysaccharide by NMR Experiments

Sizing of the polysaccharide is usually required as part of the conjugation process where sized polysaccharide, after activation is conjugated with a carrier protein (such as CRM₁₉₇, DT, TT or SCP). As mentioned earlier polysaccharide can be sized by mechanical (homogenization or sonication) or chemical process (hydrolysis). In both case the glycosidic bond is cleaved, resulting in the sizing of the polysaccharide. Overall, there should not be any structural changes upon sizing. Comparison of the 1D ¹H and 2D ¹H-¹³C HSQC spectra of native and sized polysaccharide allowed to assess whether any structural changes took place in the polysaccharide other than where the glycosidic bonds are cleaved. No structural changes were observed between the native and sized polysaccharide, except for the glycosidic bonds between B1-C4 and C1-D3 (See FIG. 4).

### Example 4: Conjugation of S. pneumoniae Serotype 22A Capsular Polysaccharide Using Direct Reductive Amination

### Mechanical Sizing of Serotype 22A Capsular Polysaccharide

The native polysaccharide was subjected to mechanical sizing under 15000 psi pressure to reduce the MW size. The sizing study was performed to identify the number of passes required to achieve the target polysaccharide MW of 125-500 kDa.

### Activating Polysaccharide

Polysaccharide oxidation was carried out in 100 mM potassium acetate buffer (pH 5.5±0.3) by sequential addition of calculated amount of 1 M potassium acetate buffer (pH 5.5) and water for injection (WFI) to give final polysaccharide concentration of 1-2 g/L. If required, the reaction pH was adjusted to pH 5.5, approximately. After pH adjustment, the reaction temperature was adjusted to 5±3° C. Oxidation was initiated by the addition of approximately 0.11 molar equivalents of sodium periodate. The oxidation reaction was performed at 5±3° C. during 16-20 hrs, approximately.

Concentration and diafiltration of the activated polysaccharide was carried out using 10K MWCO ultrafiltration cassettes. Diafiltration was performed against 20-fold diavolumes of WFI. The purified activated polysaccharide was then stored at 5±3° C. The purified activated saccharide is characterized, inter alia, by (i) saccharide concentration by colorimetric assay; (ii) aldehyde concentration by colorimetric assay; (iii) degree of oxidation; and (iv) molecular weight by SEC-MALLS.

The degree of oxidation (DO=moles of sugar repeat unit/moles of aldehyde) of the activated polysaccharide was determined as follows:

The moles of sugar repeat unit are determined by various colorimetric methods, for example, by using the Anthrone method. By the Anthrone method, the polysaccharide is first broken down to monosaccharides by the action of sulfuric acid and heat. The Anthrone reagent reacts with the hexoses to form a yellow-green colored complex whose absorbance is read spectrophotometrically at 625 nm. Within the range of the assay, the absorbance is directly proportional to the amount of hexose present.

The moles of aldehyde are also determined simultaneously, using the MBTH colorimetric method. The MBTH assay involves the formation of an azine compound by reacting aldehyde groups (from a given sample) with a 3-methyl-2-benzothiazolone hydrazone (MBTH assay reagent). The excess 3-methyl-2-benzothiazolone hydrazone oxidizes to form a reactive cation. The reactive cation and the azine react to form a blue chromophore. The formed chromophore is then read spectroscopically at 650 nm.

Compounding Activated Polysaccharide with Sucrose Excipient, and Lyophilizing

The activated polysaccharide was compounded with sucrose to a ratio of 25 grams of sucrose per gram of activated polysaccharide. The bottle of compounded mixture was then lyophilized. Following lyophilization, bottles containing lyophilized activated polysaccharide were stored at −20±5° C. Calculated amount of CRM₁₉₇ protein was shell-frozen and lyophilized separately. Lyophilized CRM₁₉₇ was stored at −20±5° C.

### Reconstituting Lyophilized Activated Polysaccharide and Carrier Protein

Lyophilized activated polysaccharide was reconstituted in anhydrous dimethyl sulfoxide (DMSO). Upon complete dissolution of polysaccharide, an equal amount of anhydrous DMSO was added to lyophilized CRM₁₉₇ for reconstitution.

### Conjugating and Capping

Reconstituted activated polysaccharide was combined with reconstituted CRM₁₉₇ in the reaction vessel, followed by mixing thoroughly to obtain a clear solution before initiating the conjugation with sodium cyanoborohydride. The final polysaccharide concentration in reaction solution was approximately 1 g/L. Conjugation was initiated by adding 1.5 MEq of sodium cyanoborohydride to the reaction mixture and incubating at 23±2° C. for 20-28 hrs. The conjugation reaction was terminated by adding 2 MEq of sodium borohydride (NaBH₄) to cap unreacted aldehydes. This capping reaction continued at 23±2° C. for 3±1 hrs.

### Purifying the Conjugate

The conjugate solution was diluted 1:5 with chilled 5 mM succinate/saline (pH 6.0) in preparation for purification by tangential flow filtration using 100K MWCO membranes.

The diluted conjugate solution was passed through a 5 μm filter, and diafiltration was performed using 5 mM succinate/saline (pH 6.0) as the medium. After the diafiltration was completed, the conjugate retentate was transferred through a 0.22 μm filter. The conjugate was diluted further with 5 mM succinate/saline (pH 6.0), to a target saccharide concentration of approximately 0.5 mg/mL.

TABLE 3
S. pneumoniae serotype 22A-CRM₁₉₇ Conjugates obtained
by Reductive Amination Conjugation (RAC) in DMSO

Conjugate Lot#

| | 1 | 2 |
|---|---|---|
| Chemistry | RAC/DMSO | RAC/DMSO |
| Poly or Sized Poly Mw, kDa | 468 | 164 (Sized) |

Poly Activation

| | | |
|---|---|---|
| NaIO4 (MEq) | 0.11 | 0.11 |
| Act Poly DO | 15 | 14 |
| Yield, % | 95 | 99 |
| Act Poly Mw, kDa | 418 | 160 |

Conjugation

| | | |
|---|---|---|
| Saccharide yield, % | 22 | 55 |
| SPR (Output Ratio) | 0.95 | 0.9 |
| Free Saccharide, % | <5 | <5 |
| Conjugate Mw, kDa | 5617 | 2688 |

Poly: Polysaccharide;
Mw: molecular weight;
MEq: Molar Equivalent;
DO: Degree of Oxidation;
Act Poly: Activated Polysaccharide
SPR: Saccharide to protein ratio

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. An isolated S. pneumoniae serotype 22A saccharide with the following repeating unit: 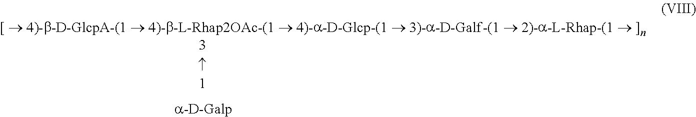
where n represents the number of repeating units and wherein the O-acetyl group at position 2 of β-D-Rhap is present in about 50% to about 100% of the repeating units.

2. The isolated S. pneumoniae serotype 22A saccharide of claim 1 having a weight average molecular weight between 5 kDa and 5000 kDa.

3. A glycoconjugate comprising an isolated S. pneumoniae serotype 22A saccharide of any one of claims 1-2 conjugated to a carrier protein.

4. A serotype 22A glycoconjugate comprising a serotype 22A capsular saccharide with the following repeating unit: 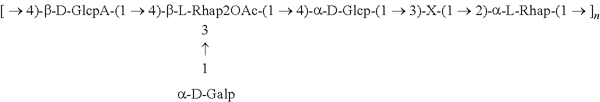
where n represents the number of repeating units, wherein X represents either a α-D-galactofuranose (α-D-Galf) or a α-L-Arabinofuranose (α-L-Araf) residue and wherein the O-acetyl group at position 2 of β-D-Rhap is present in about 50% to about 100% of the repeating units. In an embodiment, said serotype 22A capsular saccharide comprises between about 80 to about 99.5 α-D-galactofuranose (α-D-Galf) residues and between about 0.5 to about 20 α-L-Arabinofuranose (α-L-Araf) residues in every 100 saccharide repeat units of the saccharide.

5. The serotype 22A glycoconjugate of claim 4 wherein said serotype 22A glycoconjugate is prepared by direct reductive amination.

6. The serotype 22A glycoconjugate of claim 3 wherein said serotype 22A glycoconjugate is prepared by click chemistry.

7. The glycoconjugate of any one of claims 3-6 wherein the weight average molecular weight (Mw) of said S. pneumoniae serotype 22A saccharide before conjugation is between 50 kDa and 1,000 kDa.

8. The glycoconjugate of any one of claims 3-7 wherein said serotype 22A glycoconjugate has a weight average molecular weight (Mw) of between 250 kDa and 20,000 kDa.

9. The glycoconjugate of any one of claims 3-8 wherein the degree of conjugation of said serotype 22A glycoconjugate is between 2 and 15.

10. The glycoconjugate of any one of claims 3-9 wherein the ratio of serotype 22A saccharide to carrier protein in the glycoconjugate (w/w) is between 0.5 and 3.0.

11. The glycoconjugate of any one of claims 3-10 wherein said serotype 22A glycoconjugate comprises less than about 50% of free serotype 22A saccharide compared to the total amount of serotype 22A saccharide.

12. The glycoconjugate of any one of claims 3-11 between 50% and 80% of the serotype 22A glycoconjugate has a K_{d} below or equal to 0.3 in a CL-4B column.

13. The glycoconjugate of any one of claims 3-12 wherein the carrier protein of the serotype 22A saccharide glycoconjugate is selected in the group consisting of: TT, DT, DT mutants and a C5a peptidase from Streptococcus (SCP).

14. The glycoconjugate of any one of claims 3-12 wherein the carrier protein of the serotype 22A saccharide glycoconjugate is rhizavidin [aa 45-179J-GGGGSSS-SP1500-AAA-SP0785] (CP1).

15. The glycoconjugate of any one of claims 3-12 wherein the carrier protein of the serotype 22A saccharide glycoconjugate is Rhavi-linker-PdT (G294P)-linker-SP0435 [aa 62-185] fusion protein (SPP2).

16. The glycoconjugate of any one of claims 3-15 wherein the carrier protein of the serotype 22A saccharide glycoconjugate is CRM₁₉₇.

17. An immunogenic composition comprising a S. pneumoniae serotype 22A saccharide according to any one of claims 1-2.

18. An immunogenic composition comprising a S. pneumoniae serotype 22A saccharide glycoconjugate according to any one of claims 3-17.

19. The immunogenic composition of any one of claims 17-18 for use as a vaccine.

20. The immunogenic composition of any one of claims 17-18 for use in a method of preventing an infection by S. pneumoniae 22A in a subject said method comprising administering to the subject an immunologically effective amount of said immunogenic composition.


SEQUENCE LISTING (2 sequences)

SEQ ID NO: 1 - protein, 950 aa, synthetic construct
MAKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

SEQ ID NO: 2 - protein, 949 aa, synthetic construct
AKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ